(19) United States
(12) Patent Application Publication
(10) Pub. No.: US 2023/0151369 A1
(43) Pub. Date: May 18, 2023

(54) COMPOSITIONS AND METHODS FOR INHIBITING TDP-43 AND FUS AGGREGATION

(71) Applicant: THE UNIVERSITY OF BRITISH COLUMBIA, Vancouver (CA)

(72) Inventors: Neil R. Cashman, Vancouver (CA); Steven S. Plotkin, Vancouver (CA); Beibei Zhao, Vancouver (CA); Ching-chung Hsueh, Vancouver (CA); Catherine M. Cowan, Vancouver (CA)

(21) Appl. No.: 17/919,459

(22) PCT Filed: Apr. 16, 2021
(86) PCT No.: PCT/CA2021/050521

§ 371 Date: Oct. 17, 2022

Related U.S. Application Data
(60) Provisional application No. 63/011,786, filed on Apr. 17, 2020.

(51) Int. Cl. C12N 15/113; A61P 25/28

(52) U.S. Cl. CPC C12N 15/1138; A61P 25/28; C12N 2310/14; C12N 2310/315; C12N 2310/531; C12N 2310/11

(57) ABSTRACT

Disclosed herein are oligomeric compounds such as antisense oligonucleotides, siRNA and shRNAs and compositions for knocking down human RACK1, and methods for treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE) in a subject in need thereof or for reducing TDP-43 and/or FUS aggregation in a cell, the methods comprising administering to the subject in need thereof or introducing into the cell one or more antisense molecule(s) targeting RACK1, optionally one or more oligomeric compound disclosed herein.

### RELATED APPLICATION

This is a Patent Cooperation Treaty Application which claims the benefit of 35 U.S.C. § 119 based on the priority of U.S. Provisional Patent Application No. 63/011,786, filed Apr. 17, 2020 which is herein incorporated in its entirety by reference.

### INCORPORATION OF SEQUENCE LISTING

A computer readable form of the Sequence Listing “P61012PC00 Sequence Listing_ST25” (89,532 bytes), submitted via EFS-WEB and created on Apr. 16, 2021, is herein incorporated by reference.

### FIELD

The present disclosure relates to oligomeric antisense compounds for use in gene modulation of RACK1 and methods for reducing TDP-43 and FUS aggregation in disease cells. Specifically, the disclosure pertains to oligomeric antisense compounds and their use for treating TDP-43-opathy and FUS-opathy neurodegenerative diseases.

### BACKGROUND

RACK1 (Receptor for Activated C Kinase 1) is a highly conserved scaffold protein that has many normal functions, including PKC transduction, miRNA regulation, and protein translation by binding to the eukaryotic small (40S) ribosomal subunit (1). Cellular RACK1 has been reported to aggregate in cells displaying TDP43 or tau pathology (2,3).

RACK1 is a tryptophan, aspartic acid repeat (WD-repeat) protein that adopts a seven-bladed p-propeller structure. RACK1 is a core ribosomal protein of the eukaryotic 40S ribosomal subunit; a scaffold protein interacting with >100 proteins, thereby regulating a variety of signaling pathways critical for cell proliferation, transcription, protein synthesis, and neuronal functions; involved in translational regulation and ribosome quality control; and expressed in the cytosol, endoplasmic reticulum (ER), and nuclei. RACK1 is highly conserved through evolution. The amino acid sequence identity of Homo sapiens RACK1 to Mus musculus is 100%, to Rattus norvegicus is 100%, to Drosophila melanogaster is 76%, to Arabidopsis thaliana is 64%, and to Saccharomyces cerevisiae is 53% (4).

It has been reported that RACK1 interacts with wild-type and mutant huntingtin (HTT), a gene associated with Huntington's disease (10).

TAR DNA-binding protein 43 (TDP-43) is a well-known RNA/DNA binding protein involved in the pathogenesis of ALS and Frontotemporal Lobar Dementia (FTLD) (5). TDP-43 mainly localizes in the nucleus, where it participates in the expression and splicing of RNAs, whereas, when in the cytoplasm, its functions range from transport to translation of specific mRNAs (6). Binding of TDP-43 to the translational machinery is mediated by an interaction with RACK1 and that an increase in cytoplasmic TDP-43 represses global protein synthesis, an effect that is rescued by overexpression of wild-type RACK1 (2). TDP-43 represents a repressor for overall translation and its binding to polyribosomes through RACK1 may promote the formation of cytoplasmic inclusions (2). In the presence of a ribosomal binding deficient mutant (DE-RACK1) protein, nuclear localization signal-deficient (dNLS) TDP-43 protein aggregation is reduced, less associated with the translational machinery, and global translational suppression by dNLS TDP-43 is relieved (2).

Fused in Sarcoma/Translocated in Sarcoma (FUS/TLS) FUS is an RNA/DNA binding protein mainly localized in the nucleus of most cell types (6). Cytoplasmic aggregation of FUS has been reported in brain and spinal cord neurons of ALS patients with FUS mutations (6), and in ˜10% of FTLD without mutations (i.e., wild-type protein) (11).

Molecules that increase or decrease RACK1 expression have been described.

PCT/GB2007/003447 describes dopamine receptor interacting proteins as markers of disease and describes determining the presence or absence of a variant form of one or more nucleic acid sequences including in the GNB2L1 (RACK1) gene, wherein the presence of the variant is indicative of disease or susceptibility to disease.

U.S. Pat. No. 8,916,530 patent describes methods for individualized cancer therapy and mentions specific antisense/shRNA/siRNA sequences for use in knocking down upregulated RACK1 gene expression for treatment of cancer.

U.S. Ser. No. 15/844,601 describes a method for increasing the expression levels of genes including GNB2L1, by administering an agent as a cancer treatment.

PCT/EP2019/065116 describes affinity-based isolation and purification of drug-loaded extracellular vesicles, such as exosomes, wherein the exosomes are engineered to enable affinity purification.

CN101985037 describes the use of specific siRNA or antisense oligonucleotides to inhibit the RACK1 gene for treatment of tumors.

Additional treatments for TDP-43-opathies or FUS-opathies are desirable.

### SUMMARY

Disclosed herein in a first aspect is an oligomeric compound comprising a portion that is complementary to at least part of a nucleic acid target selected from any one of SEQ ID NOs: 1-16, 49-51 or 289-499.

In an embodiment, the oligomeric compound is 14 to 40 nucleotides in length.

In an embodiment, the nucleic acid target sequence is selected from any one of SEQ ID NOs: 2-6, 8, 10-16, 49-51, 292-294 and 296-499. In an embodiment, the nucleic acid target sequence is selected from any one of SEQ ID NOs: 2-6, 8, 10-16, 49-51, 292-294 and 296-298. In an embodiment, the nucleic acid target is sequence selected from any one of SEQ ID NOs: 2, 3, 292, 297 and 298.

The target sequences are in RACK1 mRNA or pre-mRNA. The sequence of human RACK1 mRNA is provided in for example NCBI Reference Sequence Accession code NM_006098.5 and having SEQ ID NO: 500. The sequence of human RACK1 pre-mRNA is provided in for example Accession code NC_000005.10 sequence index 181236897 to 181248096.

In an embodiment, the portion is complementary to the nucleic acid target sequence and the nucleic acid target sequence is or comprises a sequence selected from any one of SEQ ID NOs: 1-16, 49-51 and 289-499. In an embodiment, the portion is complementary to the nucleic acid target sequence and the nucleic acid target sequence is or comprises a sequence selected from any one of SEQ ID NOs: 2-6, 8, 10-16, 49-51, 292-294 and 296-499. In an embodiment, the portion is complementary to the nucleic acid target sequence and the nucleic acid target sequence is or comprises a sequence selected from any one of SEQ ID NOs: SEQ ID NOs: 2-6, 8, 10-16, 49-51, 292-294 and 296-298. In an embodiment, the portion is complementary to the nucleic acid target sequence and the nucleic acid target sequence is or comprises any one of SEQ ID NOs: 2, 3, 292, 297 and 298.

The oligomeric compounds can be comprised of naturally occurring or modified monomers or combinations thereof. The oligomeric compounds can be single or double stranded and can be RNA, DNA or DNA/RNA hybrids (e.g. single stranded or double stranded).

The oligomeric compound can be an antisense oligonucleotide, for example comprising the sequence of any one of SEQ ID NOs: 78-288, preferably any one of SEQ ID NOs: 81-83 and 85-87, and more preferably any one of SEQ ID NOs: 81, 86 and 87.

The oligomeric compound can be an siRNA compound that targets one of the nucleic acid targets and comprising a native or non-native overhang sequence.

In an embodiment, the siRNA comprises a guide strand that comprises a sequence of any one of SEQ ID NOs: 17-32 and 52-54.

Double stranded oligomeric compounds such as siRNA sequences can have identical 3′-overhang sequences or non-identical 3′ overhang sequences. One may be native and one may be non-native.

The oligomeric compound may be an shRNA. In an embodiment, the oligomeric compound comprises one or more cell penetrating moieties.

In a further aspect, there is disclosed a vector comprising the oligomeric compound herein disclosed.

In a further aspect, a composition comprising said oligomeric compound or vector and a diluent is disclosed.

An aspect disclosed herein relates to a method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising knocking down RACK1 RNA, optionally RACK1 mRNA and/or RACK1 pre-mRNA in cells of the central nervous system, in particular in neurons and/or astrocyte cells of a subject in need thereof.

Another aspect disclosed herein is a method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising administering to a subject in need thereof one or more antisense molecule(s), optionally one or more of said oligomeric compounds disclosed herein.

Another aspect is a method of reducing or inhibiting TDP-43 and/or FUS aggregation in a cell, the method comprising introducing into the cell one or more antisense molecule(s) optionally one or more of said oligomeric compounds targeting RACK1, compositions and/or vectors disclosed herein in a sufficient amount and for a sufficient time to decrease RACK1 levels in the cell.

A further aspect is the use of one or more antisense molecule(s), compositions, vectors and/or a methods described herein, for treating a TDP-43opathy optionally selected from amyotrophic lateral sclerosis (ALS), Alzheimer's disease (AD), frontotemporal lobar dementia (FTLD) e.g. TDP-43 type FTLD or FUS-type FTLD, Huntington's disease (HD), neuronal intermediate filament inclusion disease (NIFID), basophilic inclusion body disease (BIBD) or limbic-predominant age-related TDP-43 encephalopathy (LATE) in a subject in need thereof, or for reducing or inhibiting TDP-43 and/or FUS aggregation in a cell.

Also provided in an aspect is one or more antisense molecule(s), compositions, vectors and/or a methods described herein for use in the treatment of a TDP-43opathy optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE) in a subject in need thereof.

Further, an aspect comprises use of one or more antisense molecule(s), compositions, vectors and/or a methods described herein for the preparation of a medicament for the treatment of a TDP-43opathy optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE).

In an embodiment, the antisense molecule, optionally the oligomeric compound is an antisense oligonucleotide, an siRNA or an shRNA.

### BRIEF DESCRIPTION OF THE DRAWINGS

An embodiment of the present disclosure will now be described in relation to the drawings in which:

FIG. 1 is a series of images of cells stained for wild type and dNLS TDP-43 (HA) and RACK1.

FIG. 2 is a series of images of cells stained for wild type and different mutants of FUS(HA) and RACK1.

FIG. 3 is a series of images of cells stained for different mutants of SOD1 (SOD100) and RACK1.

FIG. 4 is a series of images of cells stained for DE-RACK1, R495x-FUS, and RACK1.

FIG. 5 is a series of images of cells stained for DE-RACK1, P525L-FUS, and RACK1.

FIG. 6A depicts the gel electrophoresis Western blotting results for surface sensing of translation which uses puromycin to tag newly synthesized protein (SUnSET). FIG. 6B depicts a graph that illustrates global translational levels normalized to a-tubulin. FIG. 6C depicts a graph that illustrates the ratio of global translational levels+/− RACK1 siRNA.

FIG. 7 is a series of images of cells stained for R495x-FUS, Puromycin (PMY), and nucleus (DAPI).

FIG. 8 is a series of images of cells stained for dNLS TDP-43, Puromycin (PMY), and nucleus (DAPI).

FIG. 9 is a series of images of cells stained for RACK1 and dNLS TDP-43+/−siRNA.

FIG. 10 is a series of images of cells stained for RACK1 and Pan TDP-43+/−siRNA.

FIG. 11 is a series of images of cells stained for RACK1 and R495x-FUS+/−siRNA.

FIG. 12 is a series of images of cells stained for RACK1 and P525L-FUS+/−siRNA.

FIG. 13 is a series of images of cells stained for RACK1 and R495x-FUS+siRNA.

FIG. 14 is a series of images of cells stained for RACK1 and Pan FUS+/−siRNA.

FIG. 15 is a series of images of cells stained for RACK1, DAPI, 40S ribosomal subunit (Rps6), and dNLS TDP-43.

FIG. 16 is a series of images of cells stained for RACK1, DAPI, 40S ribosomal subunit (Rps6), and R495x-FUS.

FIG. 17 is a series of images of cells stained for RACK1, DAPI, 40S ribosomal subunit, and P525L-FUS.

FIG. 18 is a series of images of cells stained for RACK1, DAPI, 60S ribosomal subunit (RPL14), and dNLS TDP-43.

FIG. 19 is a series of images of cells stained for RACK1, DAPI, 60S ribosomal subunit (RPL14), and R495x-FUS.

FIG. 20 is a series of images of cells stained for RACK1, DAPI, 60S ribosomal subunit (RPL14), and P525L-FUS.

FIG. 21A is a series of images of cells stained for RACK1, 40S ribosomal subunit (Rps6), and dNLS TDP-43+ RACK1 siRNA. FIG. 21B is a series of images of cells stained for RACK1, 60S ribosomal subunit (RPL14), and dNLS TDP-43+ RACK1 siRNA.

FIG. 22A is a series of images of cells stained for RACK1, 40S ribosomal subunit (Rps6), and P525L-FUS+ RACK1 siRNA. FIG. 22B is a series of images of cells stained for RACK1, 60S ribosomal subunit (RPL14), and P525L-FUS+ RACK1 siRNA.

FIG. 23 is a model of the rescue of global translation by RACK1 knockdown.

FIG. 24 is a plot of the hotspot score of siRNA prediction on RACK1 mRNA. Circle markers are the peaks of the hotspot score, and correspond to regions that has potential to be targeted by siRNA. Plus markers show the position of existing effective siRNA from literature (Table 1). Star markers correspond to the siRNA that has been made and tested herein (Table 2). Triangle markers are the negative control of the prediction (Table 5).

FIG. 25 is a series of plots depicting the hotspot score (HS) of siRNA prediction on RACK1 pre-mRNA. Here 8 exon regions are extracted, showing the intron/exon boundaries. Location 4406, 5750 and 10382 are potential splice-blocking siRNA designs (Table 4).

FIG. 26 is an image of a Western Blot testing siRNAs of Table 2 for efficacy in knocking down RACK1. Santa Cruz Biotechnology is a positive control and has the same sequences as [7].

FIG. 27 is a schematic of the UAS-Gal4 expression system used for producing flies expressing either wild-type or mutant hTDP43 or not, with or without RACK1-RNAi.

FIGS. 28A to 28L are representative photographs of fly eyes of various genotypes. GMR drives expression of transgenes, shown at A1 (FIG. 28A-28D) or at A6 (FIGS. 28E-28H, 28K, 28L). Undriven controls are shown at A6 (FIGS. 28I, 28J).

FIG. 29 is a graph showing the percentage of flies in which degeneration score remains at 1.

FIG. 30 shows Western Blotting results for the detection of RACK1 in HeLa cells treated with different ASOs. Lane loading control: tubulin

FIG. 31 is a bar graph showing RACK1 protein expression in ASO treated HeLa cells relative to untreated (UT) cells, set to 1 and represented by upper dotted line.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g. Green and Sambrook, 2012).

As used herein, the term “administration” means to provide or give a subject a compound or molecule, such as a composition comprising an antisense molecule, optionally an oligomeric compound disclosed herein or a vector comprising an antisense molecule, e.g. an shRNA by any effective route such as an intrathecal, intraventricular, intraparenchymal or intranasal administration route.

As used herein, the term “effective amount” refers to an amount of a compound or molecule, such as an antisense molecule, for example an antisense oligonucleotide or an anti-RACK1 siRNA that is sufficient to generate a desired response, such as to reduce or eliminate RACK1 protein, TDP-43 aggregation and/or FUS aggregation or to treat a TDP43-opathy or a FUS-opathy neurodegenerative disease such as amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE).

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. For example, a subject with early stage ALS or FTLD can be treated with an antisense molecule(s) such as an oligomeric compound described herein to prevent progression of disease e.g. to prevent worsening of neurodegeneration.

As used herein, the term “diluent” refers to a pharmaceutically acceptable carrier which does not inhibit a physiological activity or property of an active compound to be administered and does not irritate the subject and does not abrogate the biological activity and properties of the administered compound. Diluents include any and all solvents, dispersion media, coatings, surfactants, antioxidants, preservatives, salts, preservatives, gels, binders, excipients, disintegration agents, lubricants, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

As used herein, the term “complementarity” or “complementary” means the ability of an antisense molecule such as an oligomeric compound disclosed herein, or a portion thereof, to hybridize to the target sequence of RACK1 RNA e.g. RACK1 mRNA and/or RACK1 pre-mRNA thereby “knocking down” RACK1 (e.g. reducing RACK1 mRNA and/or pre-mRNA by at least 20%, 30%, 40%, 50%, 60%, 70%, 80%, 85%, 90% or 95% or greater). Complementarity between the antisense molecule and the target RNA may be perfect (100% complementary) but some mismatches are tolerated. For example, the antisense molecule can be 70%, 80%, 85%, 90% or 95% complementary to the target RNA or comprise up to 1, 2 or 3 mismatches in any 10 monomer stretch.

As used herein, the term “reverse complement” means the complementary strand of a nucleic acid sequence in the direction of its 5′ to 3′ end. For example, where a sequence in the 5′ to 3′ direction is TCCAGAGACAATCTGCCGGT (SEQ ID NO: 81), its reverse complement is ACCGGCAGATTGTCTCTGGA (SEQ ID NO: 292).

As used herein, “complementary to at least part” refers to an antisense molecule such as an oligomeric compound disclosed herein having sufficient complementarity to RACK1 RNA such as RACK1 mRNA or RACK1 pre-mRNA to decrease RACK1 levels, as measured for example an in vitro assay. “Complementary to at least part” includes for example complementary to at least 14, 15, 16, 17, 18, 19, 20, 21, 22, 23, 24 or 25 contiguous nucleotides of RACK1 RNA.

As used herein, the terms “antisense molecule” including for example any one of the oligomeric compounds disclosed herein comprises a compound at least a portion of which is a nucleic acid and includes for example antisense oligonucleotides, molecules comprising antisense oligonucleotides, siRNAs and molecules comprising siRNAs. The term antisense molecule includes for example antisense oligonucleotides that are typically single stranded as well as siRNA compounds which are typically double stranded as well as shRNA molecules. The antisense molecules are anti-RACK1 antisense molecules that are complementary to at least a portion of the RACK1 mRNA or pre-mRNA transcript.

As used herein, the term “oligomeric compound” relates to a compound herein disclosed that comprises an oligonucleotide, at least a portion of which is complementary to RACK1 RNA such as RACK1 mRNA or RACK1 pre-mRNA, or a part thereof. The oligomeric compound can comprise DNA, RNA, or a hybrid of DNA/RNA, and can comprise one or more modified (i.e. non-naturally occurring) monomers. “Oligomeric compound” includes antisense oligonucleotides, siRNAs and shRNA constructs. The oligomeric compound can consist of the portion that is complementary to RACK1 RNA but can also comprise additional one or more additional molecule, group or moiety (e.g. cell penetrating moiety).

As used herein, the term “antisense oligonucleotide” or “ASO” is a nucleic acid, e.g. a single stranded nucleic acid, that comprises a nucleotide sequence, which is complementary to at least a part of RACK1 RNA such as RACK1 mRNA or RACK1 pre-mRNA, and includes without limitation mixmers, gapmers, tailmers, headmers and blockmers, morpholinos, peptide nucleic acids (PNAs), 2′-O-substituted antisense oligonucleotides (e.g. 2′-O-methyl phosphorothioates, 2′-O-methoxyethyl phosphorothioates), locked nucleic acids (LNAs) and the like. Accordingly, an antisense oligonucleotide can hydrogen bond to a sense nucleic acid. For example, the antisense oligonucleotide can comprise DNA, RNA and/or a chemical analog (i.e. modified base) that binds to the target RNA.

As used herein, the term “siRNA” refers to an siRNA comprising a guide strand that is complementary to at least a part of the RACK1 mRNA or pre-mRNA transcript.

As used herein, the term “guide strand” refers to the portion or strand of an antisense molecule such as a double stranded siRNA that is complementary to the RNA sequence to which it is targeting to bind. It can comprise naturally occurring and/or modified bases. “Guide strand” can be used when referring to siRNAs and “portion” can be used when referring to antisense oligonucleotides and/or other antisense molecules.

As used herein, the term “shRNA construct” refers to a construct comprising a vector and a shDNA insert that when expressed can knock down expression of RACK1, the vector including viral vectors such as lentiviral and non-viral vectors, wherein the shDNA can be expressed to produce a short hairpin RNA comprising a guide strand that is complementary to at least a portion of the RACK1 mRNA or pre-mRNA transcript. As used herein, the term “guide strand” refers to the strand of an expressed double stranded shRNA that is complementary to the RNA sequence to which it is targeting to bind.

As used herein, the term “locked nucleic acid” or “LNA” refers to a bicyclic RNA analogue in which the ribose is locked in a C3′-endo conformation by introduction of a 2′-O,4′-C methylene bridge. Desirable LNA monomers and their method of synthesis also are disclosed in U.S. Pat. Nos. 6,043,060, 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et al., J. Org. Chem. 66(16):5498-5503, 2001; Halkansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al, Bioorg. Med. Chem. Lett. 8(16):2219-2222, 1998, all of which are herein incorporated by reference in their entirety.

The term “mixmer” refers to an antisense oligonucleotide that comprises both naturally and non-naturally occurring nucleotides. However, unlike gapmers, tailmers, headmers and blockmers, there is no contiguous sequence of more than 5 naturally occurring nucleotides.

The term “gapmer” as used herein refers to for example an antisense oligonucleotide in which an internal DNA-based region (e.g. “gap”) having a plurality of nucleosides that support RNase H cleavage is flanked by one or more RNA-based nucleosides (e.g. 5′ and 3′ “wings”) that promote target binding. The gap nucleosides are distinct from the wing nucleosides. In a non-limiting example, the gapmer comprises DNA residues flanked by 2-MOE modified RNA residues, as described in Table 8. The 5′ and 3′ wings may have the same chemical modifications however different modifications between the 5′ and 3′ wings are contemplated as well as differences in nucleotide length.

The term “morpholino oligonucleotides” as used herein refers to a non-natural oligonucleotide comprising morpholino monomers such as methylenemorpholine rings replacing the ribose or deoxyribose sugar moieties and non-ionic phosphorodiamidate linkages replacing the anionic phosphates of DNA and RNA. Antisense morpholino oligonucleotides, for example that are targeted to intronic elements can modulate RNA splicing (12). Morpholino oligonucleotides can be short chains of about 25 morpholino monomers. Each morpholino oligonucleotide would block small (˜25 base) regions of the base-pairing surfaces of ribonucleic acid (RNA). The term “morpholino monomer” refers to a subunit comprising a nucleic acid base, a 6 membered morpholine ring and a non-ionic phosphorodiamidate intersubunit linkage.

As used herein, the term “cell penetrating moiety” refers to a compound or a functional group which mediates transfer of a compound, such as an oligomeric compound herein disclosed, from an extracellular space to within a cell.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The definitions and embodiments described in particular sections are intended to be applicable to other embodiments herein described for which they are suitable as would be understood by a person skilled in the art.

As is demonstrated herein, RACK1 co-aggregates with mutant FUS and SOD1, which with mutant TDP43 could constitute a common pathway for the toxicity of these mutation-validated inclusions in for example ALS.

It is also demonstrated herein that knockdown of RACK1 in cultured cells can diminish or inhibit formation of FUS or TDP43 inclusions, accompanied by partial nuclear repatriation of mutant proteins which lack a nuclear localization sequence, perhaps due to diffusion of the de-aggregated protein into the nucleus [Pinarbasi et al., 2018]. Without wishing to be bound by theory the recruitment of polyribosomes to RACK1 co-aggregates may contribute to a toxic gain-of-function in misfolding and propagation of ALS/FTLD-implicated proteins, by virtue of recruitment of the 60s ribosomal subunit possessing the PFAR. The data described herein shows that co-aggregation of RACK1 with mutant TDP-43 or FUS suppresses global translation by sequestration of ribosomal subunits, and that siRNA knockdown of RACK1 can rescue global translation as well as the possible pathological chaperone activity of the 60s ribosome PFAR.

Neurotoxicity of protein aggregate-recruited RACK1 may be due to many factors, including loss-of-function for normal RACK1 activities. However, toxic gain-of-function of aggregated RACK1 could be one cause of the protein translational defects observed in ALS and other TDP-43 proteinopathies (i.e. TDP-43opathies).

It is also demonstrated herein that cell-specific in vivo knockdown of RACK1 ameliorates the neurodegeneration caused by transgenic overexpression of wildtype or mutant human TDP-43.

Accordingly, in an aspect is provided an oligomeric compound comprising a portion that is complementary to at least part of a nucleic acid target sequence selected from any one of SEQ ID NOs: 1-16, 49-51 and 289-499.

The portion of the oligomeric compound that is complementary to at least part of the nucleic acid target sequence can be 14, 15, 16, 17, 18, 19, 20, 21, 22, 23, 24 or 25 nucleotides in length. In an embodiment, the oligomeric compound is 14 to 60 nucleotides in length. In an embodiment, the oligomeric compound is 14 to 50 nucleotides in length. In an embodiment, the oligomeric compound is 14 to 40 nucleotides in length. In an embodiment, the oligomeric compound corresponds to the portion complementary to at least part of the target sequence and comprises 14, 15, 16, 17, 18, 19, 20, 21, 22, 23, 24 or 25 nucleotides in length. In another embodiment, the oligomeric compound includes one or more additional nucleotides in the 5′ and/or 3′ direction of the portion complementary to the target sequence. For example, the oligomeric compound can comprise up to 15 or up to 20 nucleotides upstream and downstream of the portion. In an embodiment, the oligomeric compound is 14, 15, 16, 17, 18, 19, 20, 21, 22, 23, 24 or 25 nucleotides in length.

The nucleic target sequence can be a sequence in Tables 2, 3, 4 or 8 or a part of any of the sequences therein. In an embodiment, the nucleic target sequence does not have the same sequence as a nucleic target sequence from Table 1. In an embodiment, the nucleic target sequence does not have the same sequence as a nucleic target sequence from Table 5. The oligomeric compound can be or comprise the reverse complement of a sequence in any of Tables 2, 3, 4 or 8, or a part thereof.

In an embodiment, the nucleic acid target sequence is selected from any one of SEQ ID NOs: SEQ ID NOs: 2-6, 8, 10-16, 49-51, 292-294 and 296-499.

In an embodiment, the nucleic acid target sequence is selected from any one of SEQ ID NOs: 2-6, 8, 10-16, 49-51, 292-294 and 296-298.

In an embodiment, the nucleic acid target sequence selected from any one of SEQ ID NOs: 2, 3, 292, 297 and 298.

In an embodiment, wherein the portion is complementary to the nucleic acid target sequence and the nucleic acid target sequence is or comprises a sequence selected from any one of SEQ ID NOs: 2-6, 8, 10-16, 49-51, 292-294 and 296-499.

In an embodiment, the portion is complementary to the nucleic acid target sequence and the nucleic acid target sequence is or comprises a sequence selected from any one of SEQ ID NOs: 2-6, 8, 10-16, 49-51, 292-294 and 296-298.

In an embodiment, the portion is complementary to the nucleic acid target sequence and the nucleic acid target sequence is or comprises any one of SEQ ID NOs: 2, 3, 292, 297 and 298.

In an embodiment, the portion is complementary to

(SEQ ID NO: 2)
GAACTGAAGCAAGAAGTTATC
or
(SEQ ID NO: 3)
CTCTGGATCTCGAGATAAA

In a preferred embodiment, the portion is complementary to GAACTGAAGCAAGAAGTTATC (SEQ ID NO: 2). In a preferred embodiment, the portion is complementary to CTCTGGATCTCGAGATAAA (SEQ ID NO: 3). In a preferred embodiment, the portion is complementary to SEQ ID NO: 81. In a preferred embodiment, the portion is complementary to SEQ ID NO: 86. In a preferred embodiment, the portion is complementary to SEQ ID NO: 87.

The oligomeric compound can be RNA or DNA or a hybrid thereof optionally comprising one or more modified residues. The target is RNA. Although, the targets may be represented as DNA herein, a person skilled in the art would recognize that thymidine (T) is replaced by uracil (U) in the sequences. Similarly, although an oligomeric compound may be represented as RNA herein, a person skilled in the art would recognize that the DNA compound comprises thymidine (T) instead of uracil (U).

Antisense molecules may be chemically synthesized using naturally occurring nucleotides and/or variously modified (non-naturally occurring) nucleotides designed to increase the biological stability of the molecules or to increase the physical stability of the duplex formed with the target RNA or DNA. Derivatives such as phosphorothioate derivatives and acridine substituted nucleotides can be used. Other examples of modified nucleotides can include those with N3′-P5′ phosphoramidates, 2′-deoxy-2′-fluoro-p-D-arabino nucleic acid analogue (FANA), morpholino monomers as well as those found in cyclohexene nucleic acids (CeNAs) (i.e. furanose moiety of DNA replaced by a cyclohexene ring) and tricyclo-DNA (tcDNA) (i.e. nucleotide comprising additional ethylene bridge between the centers C (3′) and C (5′) of the nucleosides, to which a cyclopropane unit is fused), peptide nucleic acid (PNA) (i.e. N-(2-aminoethyl)-glycine units), and/or be locked nucleic acid (LNA). The antisense molecule can be complementary to a target strand, or only to a portion thereof.

Antisense molecules can comprise at least one non-naturally occurring monomer which can function similarly to non-modified oligonucleotides. The chemical modification can for example be one found in locked nucleic acid (LNA) or can be 2′-fluoro (2′-F), 2′-O-methoxyethyl(2′-MOE) or 2′-O-methyl (2′-O-Me), which are modifications at the 2′ position of the ribose moiety or morpholino monomer where a six-membered morpholine ring replaces the sugar moiety or phosphorothioate (PS) linkage where sulfur replaces one of the non-bridging oxygen atoms in the phosphate group. Phosphorothioate and phosphoramidate linkages can be incorporated into any of the above-mentioned antisense molecules. Other internucleoside linkages include for example phosphorodithioate, methylphosphonate, alkylphosphonate, alkylphosphonothioate, phosphotriester, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. Such modified or substituted nucleic acids may be preferred over naturally occurring forms because of properties such as increased stability in the presence of nucleases. The term also includes chimeric nucleic acids that contain two or more chemically distinct regions. For example, chimeric nucleic acids may contain at least one region of modified nucleotides that confer beneficial properties (e.g., increased nuclease resistance, increased uptake into cells), or two or more nucleic acids of the disclosure may be joined to form a chimeric nucleic acid.

Antisense molecules can be produced using a variety of methods, for example as described in Agrawal S. & Gait M. J. (2019). History and Development of Nucleotide Analogues in Nucleic Acid Drugs. Advances in Nucleic Acid Therapeutics, (pp 1-21). Royal Society of Chemistry, incorporated herein by reference. The antisense molecules or the nucleic acid component thereof can be produced biologically using for example an expression vector introduced into cells in the form of a recombinant plasmid, phagemid or attenuated virus in which antisense sequences are produced under the control of a high-efficiency regulatory region, the activity of which may be determined by the cell type into which the vector is introduced. Additionally, antisense molecules, for example siRNA, can be purchased from manufacturers, for example Santa Cruz Biotechnology (Dallas, Tex., USA).

In another embodiment, the oligomeric compound comprises non-modified RNA, DNA or a mixture of DNA/RNA.

In an embodiment, the oligomeric compound comprises modified RNA, DNA or a mixture of DNA/RNA.

In a further embodiment, the oligomeric compound comprises one or more nucleotide monomers which is chemically modified. In a further embodiment, the chemical modification comprises modification at a 2′ position. In another embodiment, the chemical modification is selected from 2′Omethyl (2′)-O-Me), 2′-O-methoxyethyl(2′O-MOE), 2′fluoro (2′F) and 2′-0,4′-C methylene bridge i.e. locked nucleic acid monomer (LNAM).

The oligomeric compound can comprise a modified backbone. In an embodiment, the oligomeric compound comprises at least one modified occuring internucleoside linkage. In an embodiment, at least one modified internucleoside linkage is a phosphorothioate internucleoside linkage. In an embodiment, at least one internucleoside linkage is a phosphoramidate linkage. For example, all of the internucleoside linkages are phoshorothioate modified, as described for example in Example 4. Phosphorothioate linkages may be mixed Rp and Sp enantiomers, or they may be made stereoregular or substantially stereoregular in either Rp or Sp form.

In a further embodiment, the oligomeric compound comprises a modification of a plurality of nucleotide monomers. In another embodiment, all of the nucleotide monomers are modified. For example, referring to Table 8, the antisense oligonucleotides have phosphorothioate bonds between all bases and the RNA bases flanking the central DNA bases are 2′-MOE modified.

As described herein, antisense oligonucleotides of the present disclosure were found to reduce RACK1 levels in vivo.

In an embodiment, the oligomeric compound is an antisense oligonucleotide.

The antisense oligonucleotide can be DNA, RNA or a DNA/RNA hybrid thereof e.g. a mixture of DNA and RNA and can comprise one or more modified nucleotide.

In a further embodiment, the antisense oligonucleotide comprises a plurality of locked nucleic acid monomers (LNAM).

In a further embodiment, the antisense oligonucleotideis a locked nucleic acid (LNA), a LNA/DNA mixmer or a LNA/RNA mixmer.

In another embodiment, the antisense oligonucleotide is a gapmer, for example comprising a plurality of DNA nucleotides, e.g. 4, 5, 6, 7, 8, 9, 10, 11, 12, 13, 14 or 15 DNA nucleotides, flanked by a plurality of RNA nucleotides e.g. 1, 2, 3, 4, 5, 6, 7, 8, 9, or 10 RNA nucleotides, for example a gapmer described in Example 4 (Table 8).

In an embodiment, the antisense oligonucleotide comprises or is the sequence of any one of SEQ ID NOs: 78-288. In an embodiment, the antisense oligonucleotide comprises or is the sequence of any one of SEQ ID NOs: 81-83 or 85-288. In an embodiment, the antisense oligonucleotide comprises or is the sequence of any one of SEQ ID NOs: 81-83 or 85-87. In an embodiment, the antisense oligonucleotide comprises or is the sequence of SEQ ID NO: 81. In an embodiment, the antisense oligonucleotide comprises or is the sequence of SEQ ID NO: 82. In an embodiment, the antisense oligonucleotide comprises or is the sequence of SEQ ID NO: 83. In an embodiment, the antisense oligonucleotide comprises or is the sequence of SEQ ID NO: 85. In an embodiment, the antisense oligonucleotide comprises or is the sequence of SEQ ID NO: 86. In an embodiment, the antisense oligonucleotide comprises or is the sequence of SEQ ID NO: 87.

In an embodiment, the antisense oligonucleotide is a morpholino oligonucleotide.

As demonstrated herein, siRNA sequences successfully knocked down RACK1.

In an embodiment, the oligomeric compound is a small interfering RNA (siRNA).

The siRNA can comprise a guide strand that comprises the reverse complement of a sequence in any of Tables 2, 3, 4, or 8, a portion thereof or a longer sequence extending 5′ or 3′ in the RACK1 mRNA. For example, with reference to Tables 2, 3 or 4, the guide strand can comprise the reverse complement of nucleotides shown in brackets. Double-stranded antisense molecules such as siRNA can include a single stranded overhang, for example corresponding to native sequence such as the nucleotides shown in brackets in Tables 2, 3 and 4 or non-native overhangs residues. Accordingly, the siRNA can include or not include the sequence shown in brackets or it can be replaced with non-native nucleotides such as tt, or in the RNA context uu.

The target can include additional nucleotides upstream or downstream of the RACK1 target sequence. For example, the target sequence can include 2 nucleotides 5′ to the recited RACK1 target sequences, for example TTTAGAGGGAAAGATCATT (SEQ ID NO: 1) with a 5′ GA overhang, GAACTGAAGCAAGAAGTTATC (SEQ ID NO: 2) with a 5′ AT overhang, CTCTGGATCTCGAGATAAA (SEQ ID NO: 3) with a 5′ GT overhang, GCTAACTGCAAGCTGAAGA (SEQ ID NO: 4) with a 5′ TG overhang, GACAAGCTGGTCAAGGTAT (SEQ ID NO: 5) with a 5′ GG overhang, GGATGGCCAGGCCATGTTA (SEQ ID NO: 6) with a 5′ AA overhang, ACACCTTTACACGCTAGAT (SEQ ID NO: 7) with a 5′ AA overhang, CTATCTGAACACGGTGACT (SEQ ID NO: 8) with a 5′ GG overhang, CAGGGATGAGACCAACTAT (SEQ ID NO: 9) with a 5′ AC overhang, CCAACAGCAGCAACCCTAT (SEQ ID NO: 10) with a 5′ GC overhang, CTTTGTTAGTGATGTGGTT (SEQ ID NO: 11) with a 5′ CA overhang, CCCTGGGTGTGTGCAAATA (SEQ ID NO: 12) with a 5′ TA overhang, GCTGATGGCCAGACTCTGT (SEQ ID NO: 13) with a 5′ CT overhang, GATTTGTGGGCCATACCAA (SEQ ID NO: 14) with a 5′ GC overhang, GTAACCCAGATCGCTACTA (SEQ ID NO: 15) with a 5′ GG overhang, CGCAGTTCCCGGACATGAT (SEQ ID NO: 16) with a 5′ CG overhang, GTACGGACTAAGGTAGATT (SEQ ID NO: 49) with a 5′ AG overhang, TTTTACCTCCTTTAGATAA (SEQ ID NO: 50) with a 5′ TG overhang and TGTTCCCCAGGATTTAGAG (SEQ ID NO: 51) with a 5′ CC overhang, respectively. In oligomeric compounds that comprise an overhang the overhang may correspond to the reverse compliment of the residues in brackets or can be non-target residues such as tt, where undercase denotes a sequence is non-native.

In another embodiment, the guide strand is complementary to GAACTGAAGCAAGAAGTTATC (SEQ ID NO: 2) with a 5′ AT overhang, or CTCTGGATCTCGAGATAAA (SEQ ID NO: 3) with a 5′ GT overhang. In a preferred embodiment, the guide strand is complementary to GAACTGAAGCAAGAAGTTATC (SEQ ID NO: 2) with a 5′ AT overhang. In a preferred embodiment, the guide strand is complementary to CTCTGGATCTCGAGATAAA (SEQ ID NO: 3) with a 5′ GT overhang.

The overhang can for example be any 2 nucleotide combination from A, U, C, G, dA, dT, dC, dG as well as modified bases.

In an embodiment, the siRNA is or comprises a guide strand comprising a sequence 5′-3′ GAUAACUUCUUGCUUCAGUUC (SEQ ID NO: 18). In another embodiment, the sequence is 5′-3′ UUUAUCUCGAGAUCCAGAG (SEQ ID NO: 19).

In an embodiment, the siRNA is or comprises a guide strand comprising a sequence 5′ to 3′ GAUAACUUCUUGCUUCAGUUC (SEQ ID NO: 18) with an 3′ (AU) overhang (i.e. additional AU nucleotides at the 3′ end). In another embodiment, the sequence is 5′-3′ UUUAUCUCGAGAUCCAGAG (SEQ ID NO: 19) with a 3′ (AC) overhang.

In another embodiment, the siRNA is or comprises a guide strand comprising a sequence of 5′-3′ GAUAACUUCUUGCUUCAGUUC (SEQ ID NO: 18) with a 3′ (AU) overhang and/or UUUAUCUCGAGAUCCAGAG (SEQ ID NO: 19) with a 3′ (gu) overhang. In a further embodiment, the sequence is 5′-3′ GAUAACUUCUUGCUUCAGUUC (SEQ ID NO: 18) with an 3′ (AU) overhang. In another embodiment, the sequence is 5′-3′ UUUAUCUCGAGAUCCAGAG (SEQ ID NO: 19) with a 3′ (gu) overhang.

In an embodiment, the guide strand comprises GAUAACUUCUUGCUUCAGUUC (SEQ ID NO: 18) with an 3′ (AU) overhang, or UUUAUCUCGAGAUCCAGAG (SEQ ID NO: 19) with a 3′ (gu) overhang.

The siRNA can for example be single stranded or double stranded. The oligomeric compound can be double stranded for example having:

ss5′-3′ GAACUGAAGCAAGAAGUUAUC (SEQ ID NO: 34) with a 3′ (au) overhang, and
as5′-3′ GAUAACUUCUUGCUUCAGUUC (SEQ ID NO: 18) with a 3′ (AU) overhang, wherein “ss” refers here to sense strand or passenger strand and “as” refers to antisense strand which can be the guide strand. The guide strand can also be a portion thereof or include additional residues.

The siRNA can be double stranded for example having:

ss5′-3′ CUCUGGAUCUCGAGAUAAA (SEQ ID NO: 35) with a 3′ (gu) overhang; and
as5′-3′ UUUAUCUCGAGAUCCAGAG (SEQ ID NO: 19) with a 3′ (gu) overhang, wherein “ss” refers here to sense strand and “as” refers to antisense strand.

In one embodiment, the siRNA is about 21-25 residues and optionally double stranded. In one embodiment, the siRNA is 21 residues in length. In one embodiment, the siRNA is 22 residues in length. In one embodiment, the siRNA is 23 residues in length. In one embodiment, the siRNA is 24 residues in length. In one embodiment, the siRNA is 25 residues in length.

In one embodiment, the oligomeric compound is a short hairpin RNA (shRNA). Using the non-limiting example of siR-2 and siR-3, the shRNA can comprise for example;

siR-2 5′-3′:
(SEQ ID NO: 34)
GAACUGAAGCAAGAAGUUAUC
(SEQ ID NO: 18)
(loop)GAUAACUUCUUGCUUCAGUUC
siR-3 5′-3′:
(SEQ ID NO: 35)
CUCUGGAUCUCGAGAUAAA
(SEQ ID NO: 35)
(loop)UUUAUCUCGAGAUCCAGAG.

In an embodiment, the antisense molecule is comprised in a vector, for example a plasmid, or viral vector such as a lentiviral vector an adenoviral vector or an adeno associated viral (AAV) vector.

In the context of the shRNA, the loop region could be any combination of nucleotide that could form a stable loop, and normally composed of 5-10nt. The termini of the shRNA can be chemically modified and/or comprise additional overhang nucleotides.

In some embodiments, the target is a part of the sequence specified herein. For example, the target can be 19-30 nucleotides in length. In some embodiments, the portion of the oligomeric compound that is complementary to at least part of the target sequence comprises one or more alternate nucleotides. For example, the portion may comprise one or more alternate nucleotides in the 3′ half of the compound, particularly the 3′ overhang. It has been found for example that the sequence between the 5′ end and the middle of the antisense siRNA is responsible for recognizing mRNA and the middle residues (nt 10-11) are typically the cleavage site recognition.

The oligomeric compound can comprise a cell penetrating moiety, be comprised in a transport reagent, or a vector for example a recombinant plasmid or viral vector that expresses the oligomeric compound or compounds.

In an embodiment, the oligomeric compound comprises one or more cell penetrating moieties. Non limiting examples of cell penetrating moieties (or cell attaching moieties) that promote intracellular uptake include peptides e.g. Penetrin, Pip's (PMO/PNA internalization peptide), sugars e.g. N-acetylgalactosamine (GaINAc), antibodies, e.g. a Fab fragment, carbohydrates, lipids e.g. cholesterol, phospholipids, biotin, phenazine, folate, phenanthridine, anthraquinone, acridine, fluoresceins, rhodamines, coumarins, and dyes. The cell penetrating moiety can be operably linked or conjugated to the 5′ end, the 3′ end and/or to internal nucleotides of the portion of the oligomeric compound that is complementary to the target sequence. In an embodiment, the cell penetrating moiety is conjugated to the 5′ end and/or the 3′ end. In the context of a double stranded siRNA, the cell penetrating moiety is preferably attached to the passenger strand, for example at the 3′ terminus. The oligomeric compound can be coupled to the cell penetrating moiety using a variety of methods. For example, the oligomeric compound can be covalently linked to the moiety, as described for example in International patent application publication no. WO2008/063113 to Langel et al. and United States patent application publication no. US2005/0260756 to Troy et al. The moiety can also be linked to the oligomeric compound via chemical linkers, as described for example in WO2008/033285 to Troy et al and WO2007/069068 to Alluis et al.

Another aspect is a vector comprising the oligomeric compound or the portion thereof that is complementary to at least part of the target sequence. For example, the oligomeric compound is comprised in a viral vector such as an adeno-associated virus (AAV), an adenovirus, a lentivirus, or a γ-retroviral vector. The vector can be an integrating vector optionally for providing constitutive expression or can be an extranuclear vector optionally for transient expression.

Another aspect is a composition comprising an oligomeric compound, optionally an anti-RACK1 siRNA, anti-RACK1 shRNA construct, or an antisense oligonucleotide (e.g. anti-RACK1 gapmer or morpholino oligonucleotide) and a diluent. The diluent can for example be RNase free water or saline, optionally sterile.

The composition can comprise lipid particles such as liposomes, nanoparticles, exosomes, or nanosomes for delivering the antisense molecules.

As mentioned above the antisense molecules can be comprised in a vector. The vector can for example be a plasmid, bacterial or viral vector such as lentiviral particles or AAV. The composition can comprise multiple oligomeric compounds and/or other antisense molecules, for example for targeting RACK1.

The compositions described herein can be prepared by per se known methods for the preparation of pharmaceutically acceptable compositions that can be administered to subjects, optionally as a vaccine, such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the subject.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylarnino ethanol.

The compositions, oligomeric compounds and vectors described herein can be formulated for example for intrathecal, intraventricular, intracranial, intraspinal, intraorbital, ophthalmic, intracisternal, intraparenchymal, intraperitoneal, intranasal, aerosol or oral administration. In a preferred embodiment, compositions, oligomeric compounds and vectors are formulated for intrathecal administration.

Also provided in another aspect is a method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising knocking down RACK1 in cells of the central nervous system such as neurons and/or astrocyte cells of a subject in need thereof.

The “knocking down” can be achieved using an antisense molecule, such as an oligomeric compound described herein, targeting RACK1 mRNA and/or pre-mRNA.

Also provided in another aspect is a method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising administering to a subject in need thereof one or more antisense molecule(s), for example one or more oligomeric compound disclosed herein.

Also provided in another aspect is a method of reducing or inhibiting TDP-43 and/or FUS aggregation in a cell such as a disease cell comprising TDP-43 and/or FUS aggregation, the method comprising administering to the cell or introducing into the cell one or more antisense molecule(s) targeting RACK1 in a sufficient amount and for a sufficient time to decrease RACK1 levels in the cell. In one embodiment, the amount and/or time is sufficient to reduce TDP-43 aggregation and/or partially restore nuclear TDP-43. In one embodiment, the amount and/or time is sufficient to reduce FUS aggregation and/or partially restore nuclear FUS.

The antisense molecules, for example the oligomeric compounds of the present disclosure, may be administered alone, as naked antisense molecules. As used herein “naked” means that the antisense molecule is not administered using a delivery vehicle (e.g. viral vector) or delivery agent (e.g. liposome) e.g. viral vector, transport reagent.

In one embodiment, the antisense molecule(s) is/are administered and/or introduced into the cell via with a transport reagent, as a recombinant plasmid or as a viral vector that expresses the antisense molecule(s). In a further embodiment, the antisense molecules(s) are introduced into the cell via electroporation.

In another embodiment, the antisense molecule(s) comprise one or more cell penetrating moieties. In such context, the antisense molecule can be injected alone i.e. naked, for example intrathecally, and other elements of the antisense molecule are relied upon, e.g. chemical modification(s), for facilitating delivery into the cell. In another embodiment, the one or more antisense molecule is an antisense oligonucleotide, an siRNA, or an shRNA construct. In another embodiment, the antisense molecule(s) is one or more of the aforementioned oligomeric compounds.

In other embodiments, the one or more antisense molecules further targets a nucleic acid target sequence listed in Table 1.

For example, the one or more antisense molecule is an antisense oligonucleotide molecule disclosed herein, for example comprising or consisting of any one of SEQ ID NOs: 81, 86 or 87.

For example, the one or more antisense molecules can be an siRNA molecule, for example comprising sense 5′-CCUUUACACGCUAGAUGGU (SEQ ID NO: 501) with a 3′ tt overhang and antisense 5′-ACCAUCUAGCGUGUAMGG (SEQ ID NO: 502) with a 3′ tg targeting CCTTTACACGCTAGATGGT (SEQ ID NO: 75).

In another embodiment, the one or more antisense molecule(s) is introduced via the aforementioned composition.

In an embodiment, the cell is a diseased cell. In an embodiment, the cell is a cell of the central nervous system such as a neuron or an astrocyte. In an embodiment, the cell is in a subject, with a TDP43-opathy or a FUS-opathy neurodegenerative disease such as amyotrophic lateral sclerosis (ALS), frontotemporal lobar dementia (FTLD) proteinopathies or a protein folding disease where the disease protein interacts with RACK1. For example, the TDP43-opathy is amyotrophic lateral sclerosis (ALS), Alzheimer's Disease (AD), frontotemporal lobar dementia (FTLD), Huntington's Disease (HD) or limbic-predominant age-related TDP-43 encephalopathy (LATE). In another embodiment, the FUS-opathy neurodegenerative disease is neuronal intermediate filament inclusion disease (NIFID) or basophilic inclusion body disease (BIBD).

In another embodiment, the one or more antisense molecule(s) is the aforementioned oligomeric compound and/or is comprised in the aforementioned composition. In an embodiment, the antisense molecule and/or composition is administered or introduced into a cell together with a transport reagent, or as a recombinant plasmid or viral vector that expresses the antisense molecule. The transport reagent can be lipid particles such as liposomes, nanoparticles, or nanosomes. In an embodiment, the transport reagent is a liposome.

In another embodiment, the antisense molecule and/or composition is administered in a suitable parenteral or enteral route of administration, including intranasal, mucosal, oral, sublingual, transdermal, topical, inhalation, aerosol, intraocular, intratracheal, intrarectal, vaginal, by gene gun, dermal patch, eye drop or mouthwash form or intravascular administration; in particular intrathecal, intraventricular, intraparenchymal or intracerebroventricular administration; e.g., a catheter or other placement device for example using an implanted reservoir that is connected to the ventricles within the brain or spinal cord via an outlet catheter.

In other embodiments, the pharmaceutical composition is administered directly to the brain or other portion of the CNS. For example, such methods include the use of an implantable catheter and a pump, which would serve to discharge a pre-determined dose through the catheter to the infusion site. A person skilled in the art would further recognize that the catheter may be implanted by surgical techniques that permit visualization of the catheter so as to position the catheter adjacent to the desired site of administration or infusion in the brain. Such techniques are described in Elsberry et al. U.S. Pat. No. 5,814,014 “Techniques of Treating Neurodegenerative Disorders by Brain Infusion”, which is herein incorporated by reference. Also contemplated are methods such as those described in US patent application 20060129126 (Kaplitt and During “Infusion device and method for infusing material into the brain of a patient”. Devices for delivering drugs to the brain and other parts of the CNS are commercially available (eg. SynchroMed® EL Infusion System, Medtronic, Minneapolis, Minn.).

In another embodiment, the pharmaceutical composition is administered to the brain using methods such as modifying the compounds to be administered to allow receptor-mediated transport across the blood brain barrier.

Other embodiments contemplate the co-administration of the antisense molecules with biologically active molecules known to facilitate the transport across the blood brain barrier.

Also contemplated in certain embodiments, are methods for administering antisense molecules described herein across the blood brain barrier such as those directed at transiently increasing the permeability of the blood brain barrier as described in U.S. Pat. No. 7,012,061 “Method for increasing the permeability of the blood brain barrier”, herein incorporated by reference.

When the route of administration is oral, the pharmaceutical composition can be in the form of a tablet, capsule, powder, solution or elixir. When administered in tablet form, the pharmaceutical composition may additionally contain a solid carrier such as a gelatin or an adjuvant. The tablet, capsule, and powder contain from about 5 to 95% antisense molecule and preferably from about 25 to 90% antisense molecule. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution or glycols such as ethylene glycol, propylene glycol or polyethylene glycol. When administered in liquid form, the pharmaceutical composition contains from about 0.5 to 90% by weight of the antisense molecule or from about 1 to 50% antisense molecule.

Where the administration is parenteral, mucosal delivery, oral, sublingual, transdermal, topical, inhalation, intranasal, aerosol, intraocular, intratracheal, intrarectal, vaginal, by gene gun, dermal patch or in eye drop or mouthwash form, the antisense molecule can be in the form of a pyrogen-free, parenterally acceptable aqueous solution, and may, in addition to the antisense molecule(s), contain an isotonic vehicle such as Sodium Chloride Injection, Ringer's Injection, Dextrose Injection, Dextrose and Sodium Chloride Injection, Lactated Ringer's Injection or other vehicle as known in the art. The pharmaceutical composition may also contain stabilizers, preservatives, buffers, antioxidants or other additives known to those of skill in the art.

The amount of antisense molecule in the pharmaceutical composition will depend upon the nature and severity of the condition being treated, and on the nature of prior and concurrent treatments which the subject has undergone or is undergoing. It is contemplated that the various pharmaceutical compositions used to practice the presently disclosed method may comprise about 1 micrograms to about 50 mg of antisense molecule per kg body per day. The duration of the treatment with the pharmaceutical composition herein disclosed will vary, depending on the disease, severity of the disease and the condition and potential idiosyncratic response of each individual subject.

In another embodiment, the TDP43-opathy neurodegenerative disease is amyotrophic lateral sclerosis (ALS), Alzheimer's Disease (AD) or frontotemporal lobar dementia (FTLD), or limbic-predominant age-related TDP-43 encephalopathy (LATE). In another embodiment, the FUS-opathy neurodegenerative disease is neuronal intermediate filament inclusion disease (NIFID) or basophilic inclusion body disease (BIBD).

In another embodiment, the subject is a human.

Another aspect is the use of one or more antisense molecules, for example the aforementioned oligomeric compounds such as antisense oligonucleotide(s) or siRNA molecule(s), and/or the methods, to treat amyotrophic lateral sclerosis (ALS), Alzheimer's disease (AD), frontotemporal lobar dementia (FTLD) or Huntington's disease (HD) in a subject in need thereof, or to reduce and/or disaggregate TDP-43 and/or FUS in a cell such as a diseased cell.

Another aspect is one or more antisense molecules, for example oligomeric compounds herein disclosed for use in the treatment of a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE).

In an embodiment is use of the aforementioned anti-RACK1 antisense molecules including the oligomeric compounds, such as antisense oligonucleotides, siRNA molecule(s) and/or composition for use in the manufacture of a medicament.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

Knockdown of RACK1 in cultured cells can diminish or inhibit aggregation of FUS and TDP43 mutants, which is accompanied by partial nuclear repatriation of mutant proteins lacking a nuclear localization sequence.

Herein, is data showing that co-aggregation of RACK1 with TDP43 or FUS suppresses global translation by sequestration of ribosomal subunits, and that siRNA knockdown of RACK1 can rescue global translation and prevent TDP-43 mediated neurodegeneration.

### Example 1

Human embryonic kidney 293T (HEK293T) cell line was purchased from American Type Culture Collection (ATCC, Rockville, Md.), and maintained in Dulbecco's Modified Eagle Medium (DMEM) supplemented with 10% fetal bovine serum (FBS), GlutaMax™-1 (2 mM) and antibiotics (50 U/ml penicillin and 50 mg/ml streptomycin) at 37 C in 5% CO₂. HEK293T cells were transfected with HA-tagged dNLS TDP-43, R495x-FUS, or P525L-FUS cDNA plasmid using Lipofectamine LTX reagent (ThermoFisher Scientific) following the manufacturer's instruction, and cells were analyzed 48 hrs post-transfection.

RACK1 knockdown was achieved by introducing a pool of 3 19-25 nucleotide siRNAs specifically targeting human RACK1 (Santa Cruz Biotechnology, sc-36354) with Lipofectamine RNAiMAX transfection reagent (ThermoFisher Scientific) and incubated for 72 hrs according to the manufacturer's instruction, followed by transfection of cDNA plasmids of HA-tagged dNLS TDP-43, R495-FUS, or P525L-FUS as described above.

Surface Sensing of Translation (SUnSET) was performed to monitor global translation. 48 hrs post-cDNA transfection, cells were incubated with 5 μg/ml of puromycin (ThermoFisher Scientific) in conditioned media for 10 min at 37 C, immediately followed by immunocytochemical or biochemical procedures.

Immunocytochemistry (ICC) was performed to visualize the expressions of HA-tagged dNLS TDP-43, R495x-FUS, P525L-FUS, SOD1 mutants, RACK1, and global protein translation. Cells were washed twice with Phosphate Saline Buffer (PBS) and fixed in 4% paraformaldehyde (PFA) for 15 min at room temperature (RT), followed by wash with 20 mM glycine for 10 min at RT with constant rocking. Cells were then incubated with blocking buffer containing PBS, 1% Bovine Serum Albumin (BSA), 10% normal goat serum, and 0.1% Triton-X-100 for 30 min at RT. The following primary antibodies were incubated for 1 h at RT or overnight at 4 C: rabbit polyclonal anti-HA (Abcam, ab9110, 1:1000), chicken polyclonal anti-HA (Abcam, ab9111, 1:8,000), mouse monoclonal anti-RACK1 (BD Biosciences, 610178, 1:500), and mouse monoclonal anti-puromycin (ThermoFisher Scientific, clone 12D10, 1:1000). Cells were then washed with PBS/0.1% Triton-X-100 3×10 min with constant rocking, followed by incubation with Alexa Fluor® goat anti-rabbit, -mouse, or -chicken secondary antibody (ThermoFisher Scientific, 1:1000) for 30 min at RT in the dark. Cells were then washed with PBS/0.1% Triton-X-100 3×10 min, dipped in 5% PBS, and mounted with ProLong Gold Anti-fading mounting media with DAPI (ThermoFisher Scientific, P36931). Cells were analyzed by confocal microscopy (Leica TCS SP8 MP).

To quantify global translational levels, following SUnSET described above, cells were washed twice with cold PBS, and lysed in 2% SDS followed by sonication at 30% power for 15 sec to extract total protein. Protein concentration was determined by BCA assay (ThermoFisher Scientific). 10 pg of protein from each transfection was separated on 4-12% NuPAGE SDS-PAGE (ThermoFisher Scientific), transferred onto a PVDF membrane, and blocked in Tris buffered saline (TBS) containing 5% skim milk and 0.1% Tween-20 for 1 h at RT. The following primary antibodies were incubated overnight at 4 C: rabbit anti-HA (Abcam, ab9110, 1:1000), mouse anti-RACK1 (BD Biosciences, 610178, 1:2000), mouse anti-puromycin (ThermoFisher Scientific, clone 12D10, 1:10,000), mouse anti-a-tubulin (ProteinTech, 66031-1-Ig, 1:20,000). Membranes were washed with TBS/0.1% Tween (TBST) 3×10 min at RT with constant rocking, followed by horseradish peroxidase (HRP)-conjugated anti-mouse or anti-rabbit secondary antibody (GE, 1:5000) incubation for 30 min at RT. Membranes were then washed with TBST 3×10 min, and developed with SuperSignal™ West Femto Maximum Sensitivity Substrate (ThermoFisher Scientific).

### Results

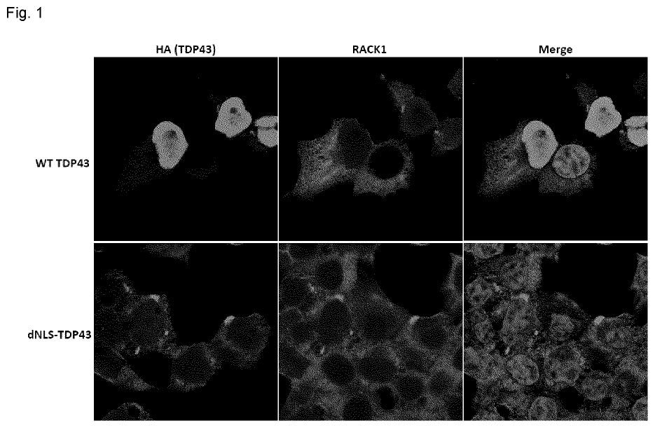

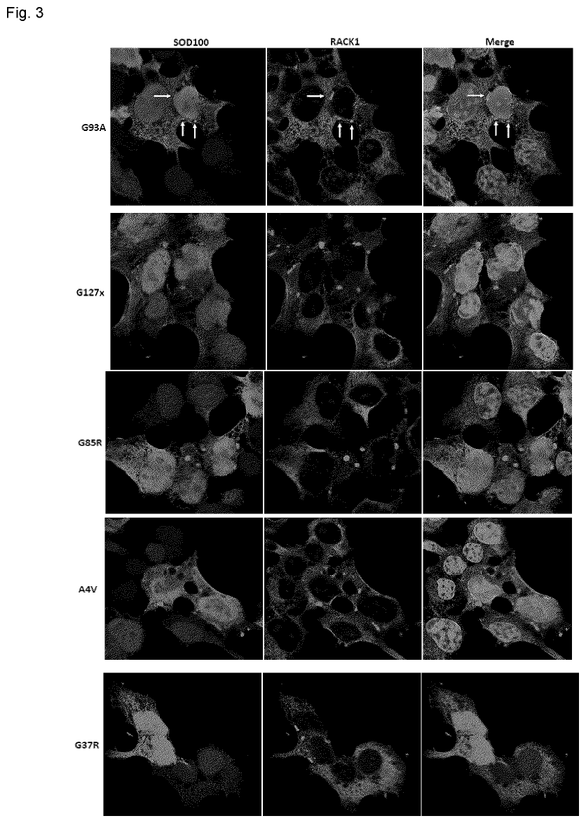

Using the methods described herein, it is demonstrated that cytoplasmic aggregates of dNLS TDP-43 induce RACK1 aggregation and co-aggregation (FIG. 1) and dNLS TDP-43 aggregates suppress global translation in transfected cells (FIG. 8). It is further demonstrated that cytoplasmic aggregates of mutant SOD1 induce RACK1 aggregation and co-aggregation (FIG. 3).

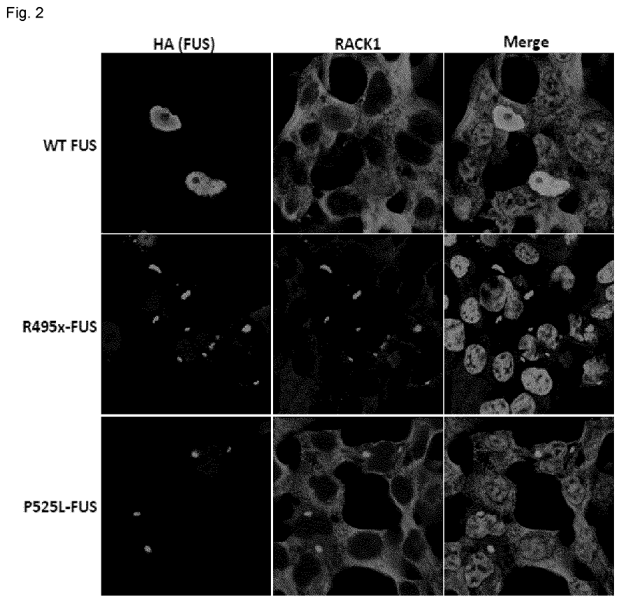

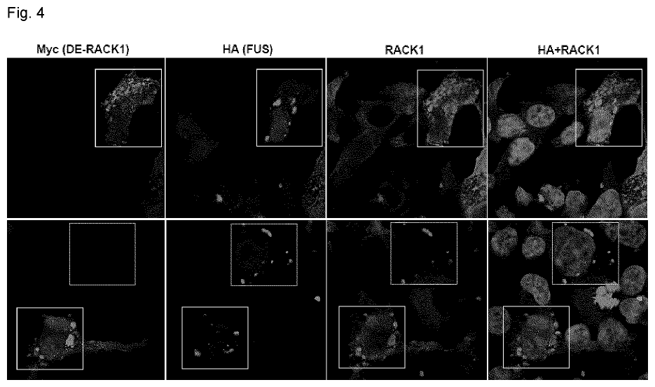

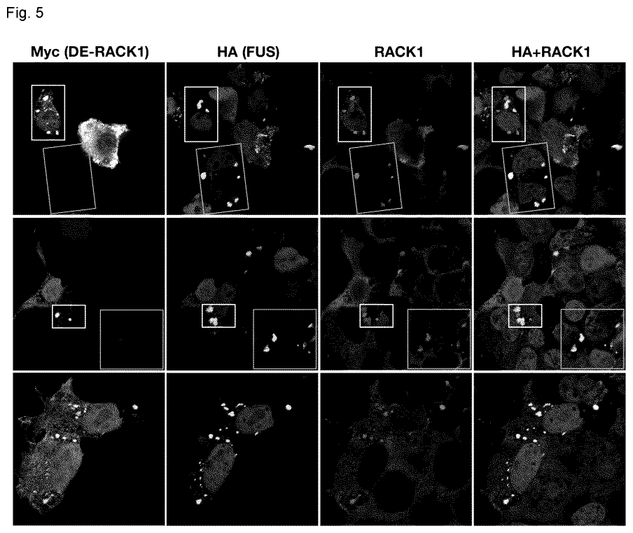

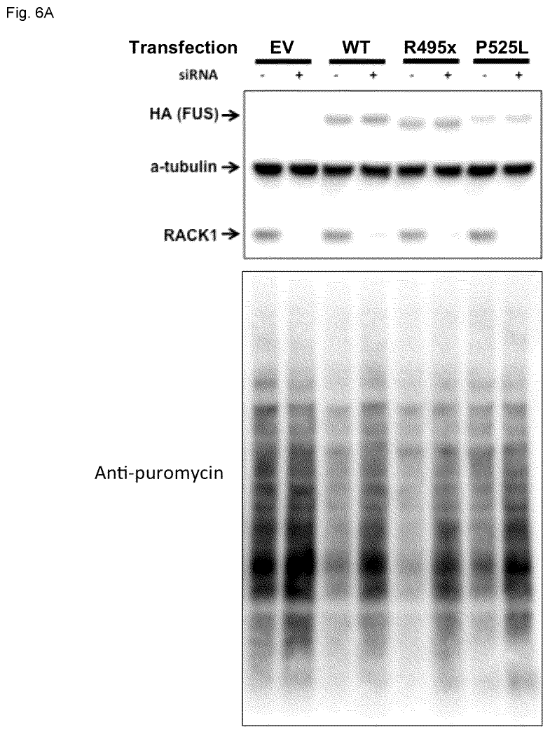

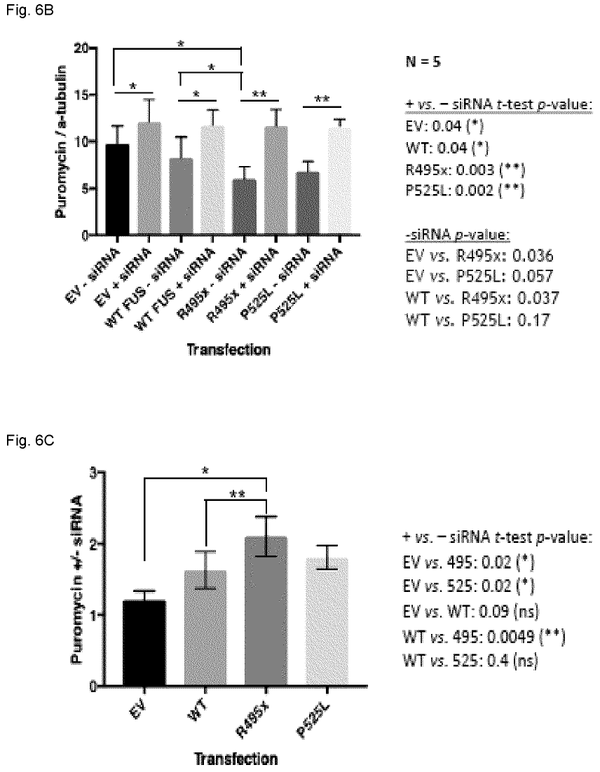

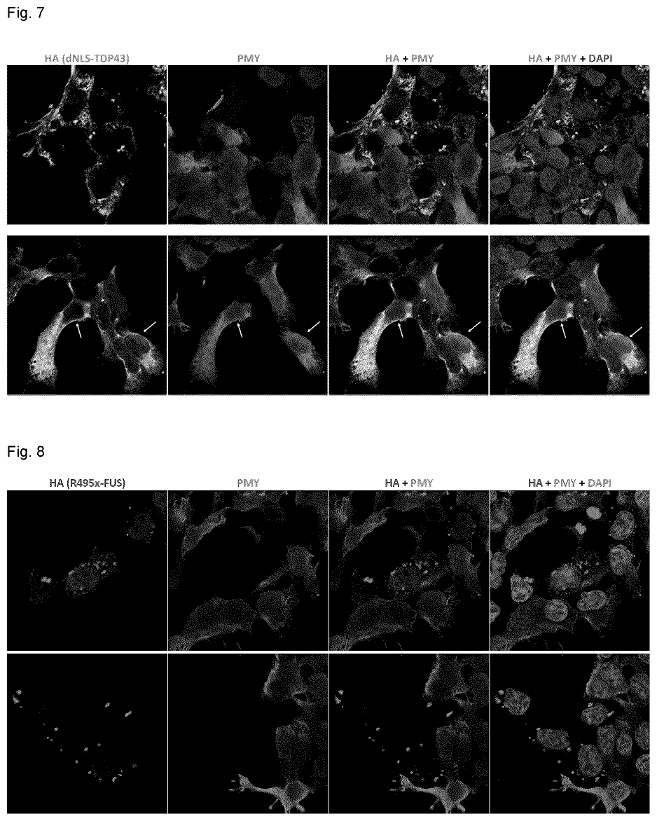

It is demonstrated that cytoplasmic aggregates of dNLS FUS, R495x-FUS and P525L-FUS, induce RACK1 aggregation and co-aggregation (FIG. 2), and dNLS-FUS transfected individual cells demonstrate global translational suppression (FIG. 7). It is also demonstrated that ribosomal binding deficient mutant (DE-RACK1) disrupts mutant FUS, R495x-FUS, and RACK1 co-aggregation (FIG. 4) and partially disrupts P525L-FUS and RACK1 co-aggregation (FIG. 5). It is further demonstrated that mutant FUS suppresses global translation, which can be rescued by RACK1 knockdown (FIGS. 6A, 6B, and 6C).

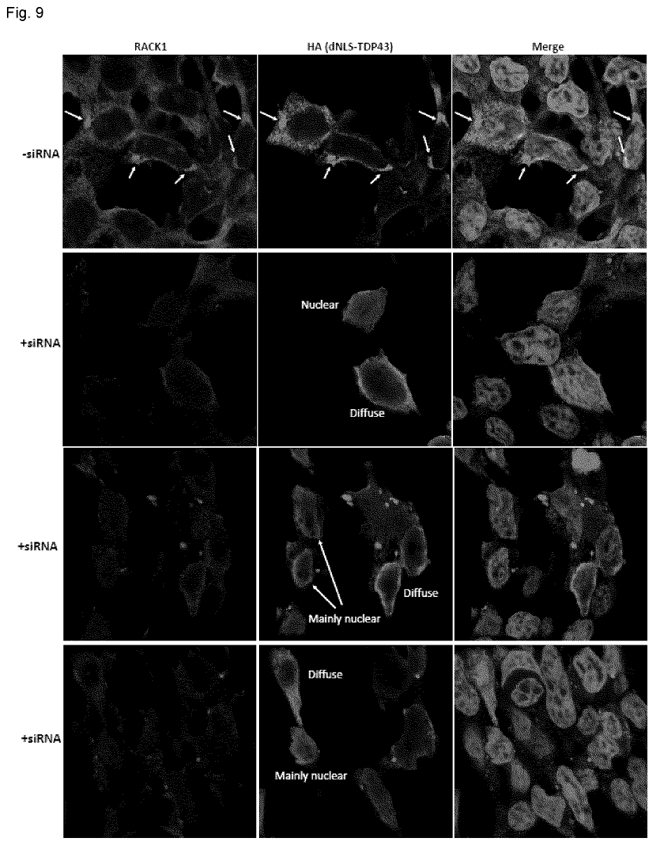

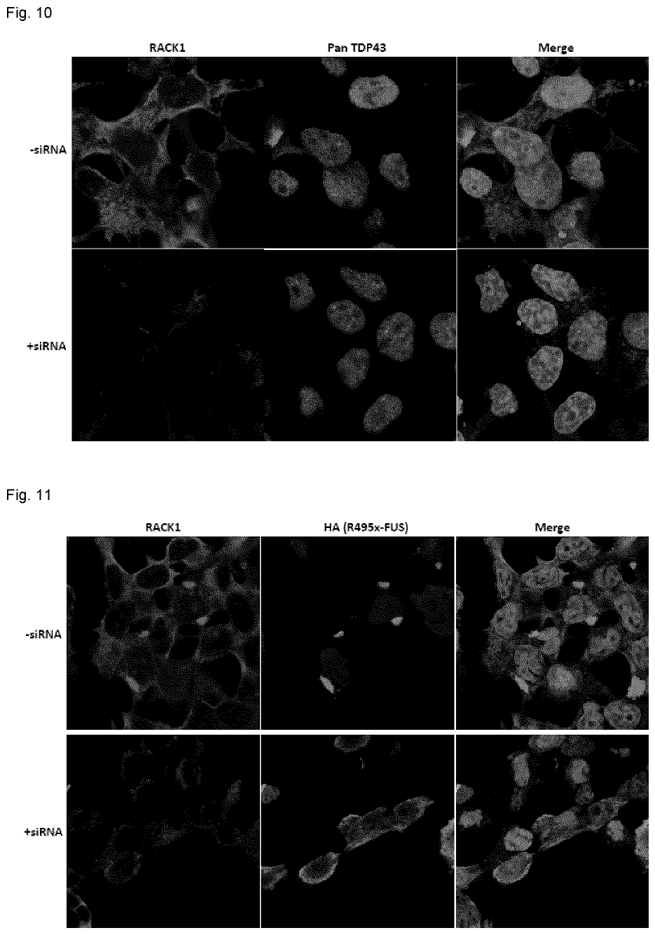

siRNA targeted to RACK1 (RACK 1 siRNA) knocks down RACK1 and attenuates dNLS TDP-43 aggregation in the cytoplasm and partially restores nuclear expression (FIG. 9), while it does not affect endogenous nuclear TDP43 expression in empty vector transfected cells (FIG. 10).

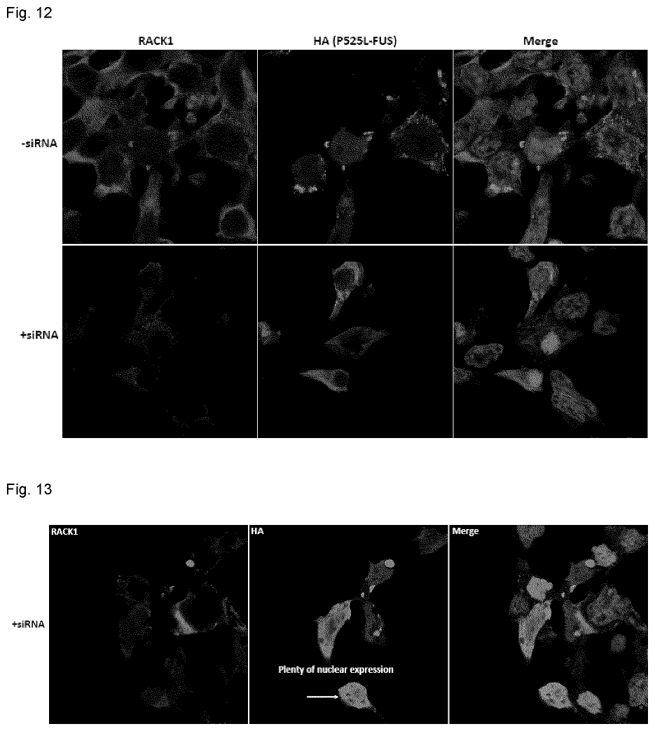

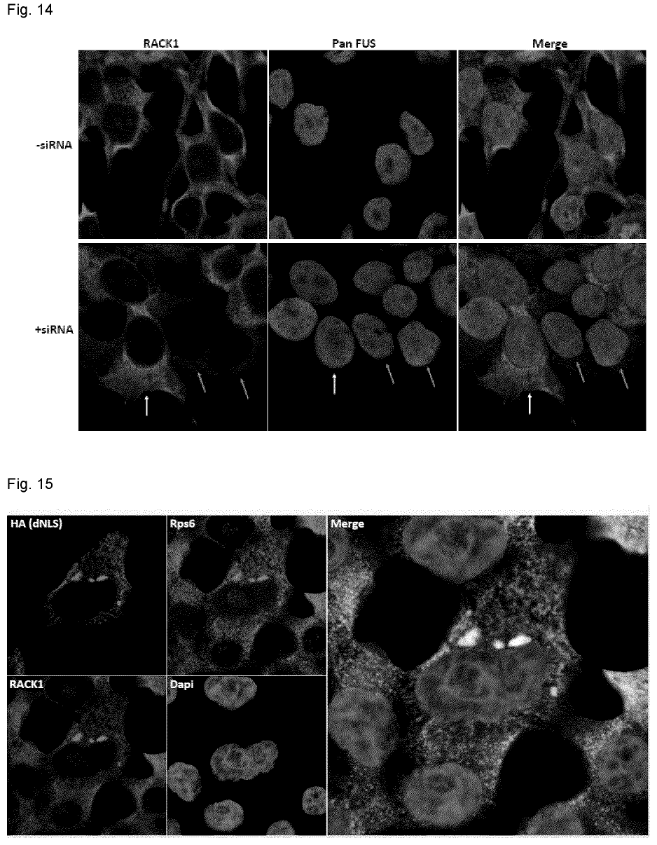

RACK1 siRNA attenuates mutant FUS, R495x-FUS(FIG. 11) and P525L-FUS(FIG. 12), aggregation in the cytoplasm, and partially restores the nuclear expression of mutant FUS, R495x-FUS(FIG. 13). RACK1 siRNA does not affect endogenous nuclear FUS expression in empty vector transfected cells (FIG. 14).

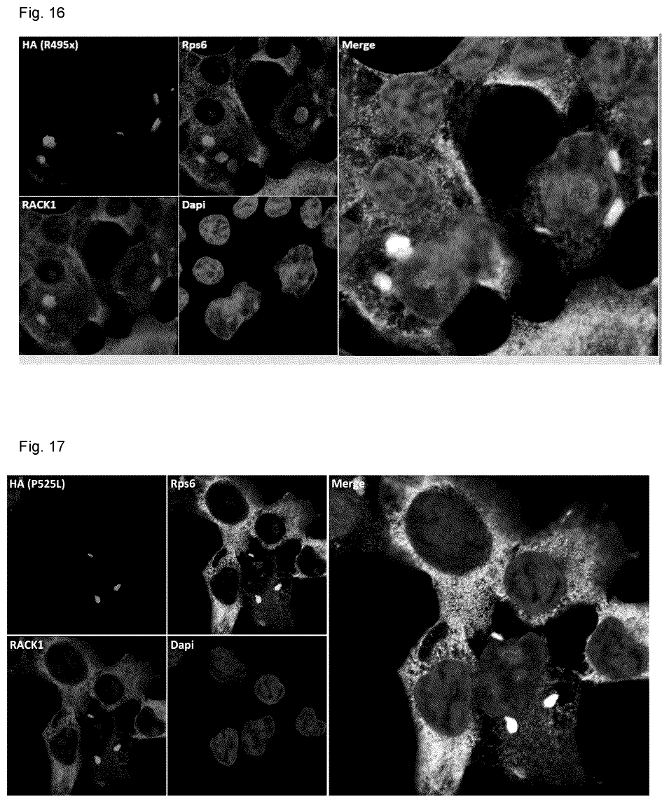

dNLS TDP-43, RACK1, and 40S (small ribosomal subunit, Rps6 as marker) co-aggregate (FIG. 15), dNLS R495x-FUS, RACK1, and 40S co-aggregate (FIG. 16), and dNLS P525L-FUS, RACK1, and 40S co-aggregate (FIG. 17). Additionally, dNLS TDP-43, RACK1, and 60S (large ribosomal subunit, RPL14 as marker) co-aggregate (FIG. 18), dNLS R495x-FUS, RACK1, and 60S co-aggregate (FIG. 19), and dNLS P525L-FUS, RACK1, and 60S co-aggregate (FIG. 20).

Upon RACK 1 knockdown, “rescued” nuclear dNLS TDP-43 (FIGS. 21A and 21B) or dNLS FUS, P25L-FUS(FIGS. 22A and 22B), does not associate with either ribosomal subunit. Where dNLS TDP-43 (FIGS. 21A and 21B) or dNLS FUS, P525L-FUS(FIGS. 22A and 22B), does remain in the cytoplasm, it often displays a more diffused pattern, as opposed to the typical large aggregates, and remains interacting with the ribosome.

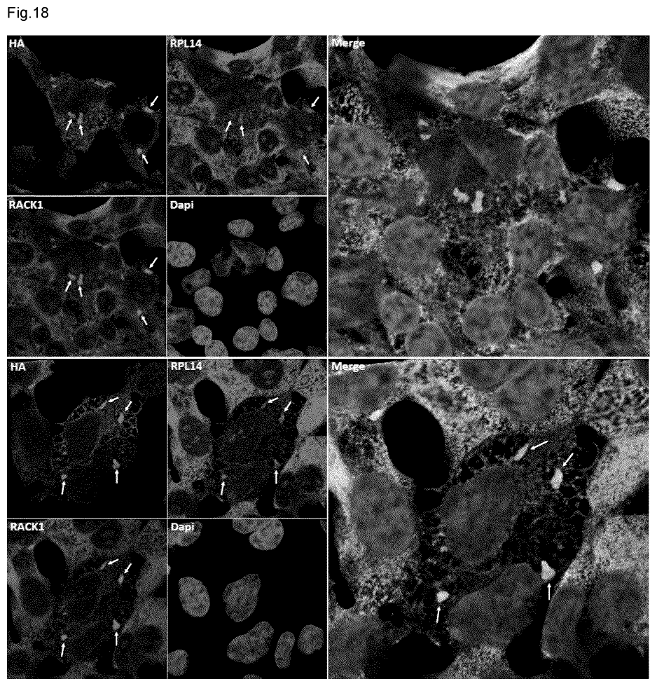

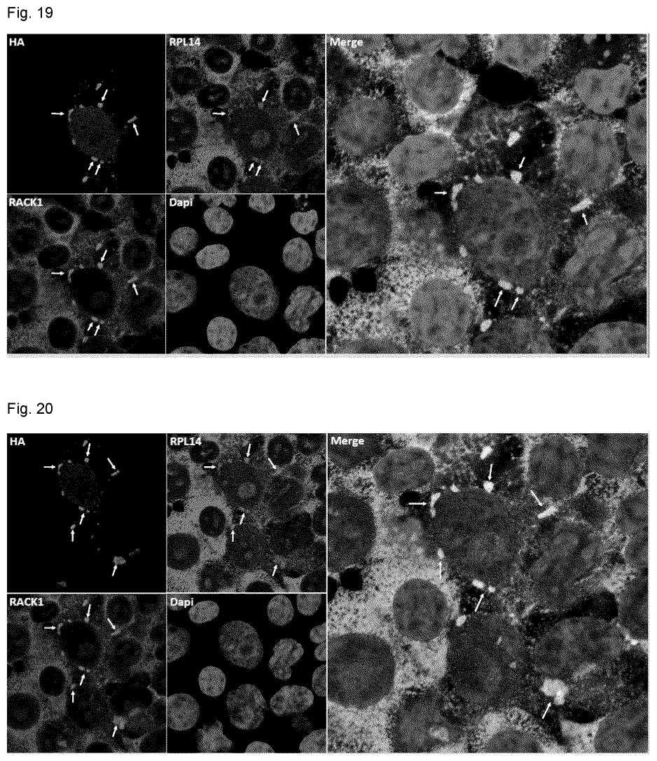

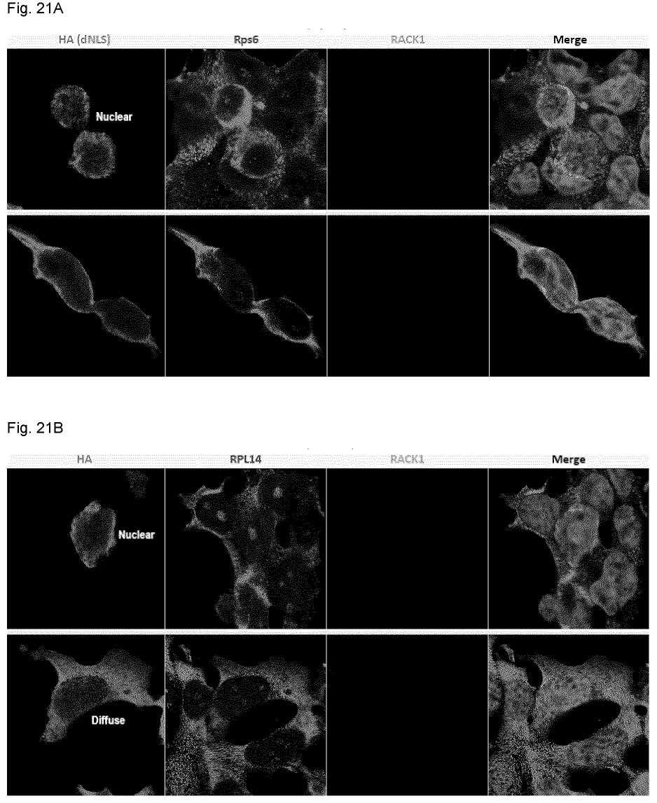

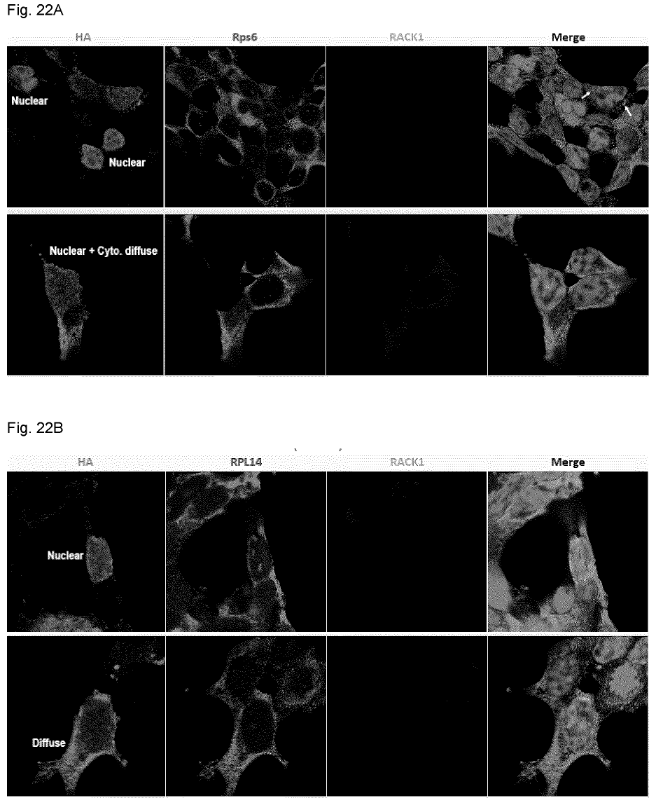

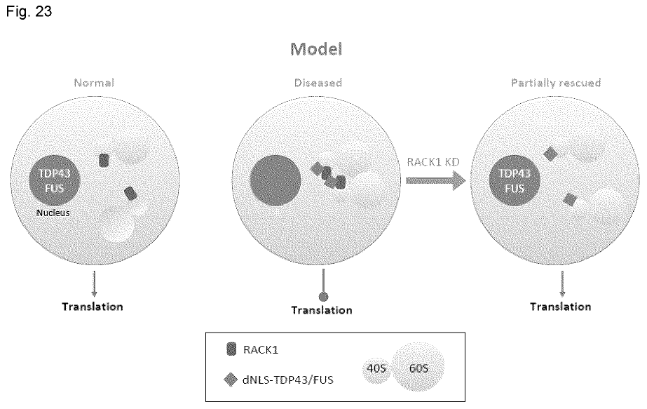

dNLS FUS or TDP 43 and RACK1 co-aggregates sequester polyribosome 40S and 60S subunits, resulting in global translational suppression (FIGS. 15-20). RACK1 knockdown disperses dNLS FUS or TDP-43 aggregates in the cytoplasm, or even restores their nuclear expressions in a proportion of cells, which as a result releases polyribosomes from the aggregates and rescues global translation (FIGS. 21-23). SUnSET ICC shows that, unlike dNLS TDP-43 aggregates, filamentary/diffuse dNLS TDP-43 expressing cells display normal global translation (FIG. 8). This data suggests that knocking down RACK1 presents a great potential to normalize pathological TDP 43/FUS aggregates and translational machinery function without affecting endogenous nuclear TDP-43, which makes RACK1 an extremely attractive therapeutic target for ALS and FTLD.

### Example 2

siRNAs were designed targeting RACK1 mRNA using the following method.

Step 1. The siRNA meta-prediction result was collected from five servers (listed below). For the starting position of the candidate siRNA, a server-based prediction score is recorded for the 5 servers. The score definitions for each of the servers are different, and defined as follows.

BLOCK-It™ RNAi Designer tool by Thermo Fisher: Gives the quality of prediction as zero to five stars (0-5) with an interval of half star (Link: https://rnaidesigner.thermofisher.com/rnaiexpress/setOption.do?designOption=sirna). Score was normalized to a max score of unity with an interval 0.1.

The RNAi design tool of siDirect: This server gives a binary yes/no prediction, which is given a score of one or zero (1 or 0) for each start position in the sequence. (Link: http://sidirect2.rnai.jp/design.cgi)

OligoWalk siRNA design tool of Mathews Lab at University of Rochester Medical Center: This server gives a continuous probability between 0 and 1 for a given sequence to be an efficient siRNA (Link: http://rna.urmc.rochester.edu/cgi-bin/server_exe/oligowalk/oligowalk_form.cgi). This probability is directly converted to a score.

siRNA wizard design tool of Invivogen: This server categorizes their prediction into either effective siRNA, moderate siRNA, or ineffective siRNA when no prediction is made (Link: https://www.invivogen.com/sirnawizard/design advanced.php). These categories are converted to scores of 1, 0.5, or 0 respectively.

siRNA target finder of Genescript: This server gives an unnormalized score for each prediction (Link: https://www.genscript.com/tools/sirna-target-finder). The score values were subsequently normalized to unity by dividing by the maximum prediction score.

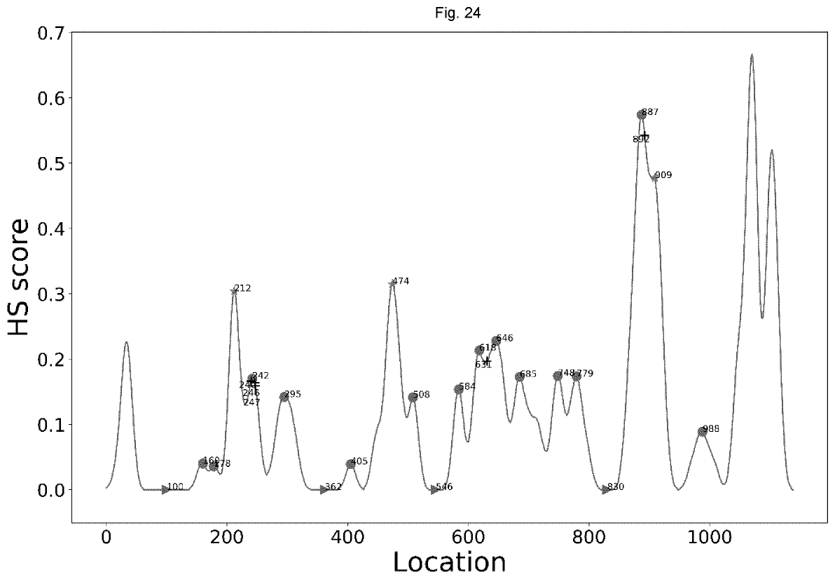

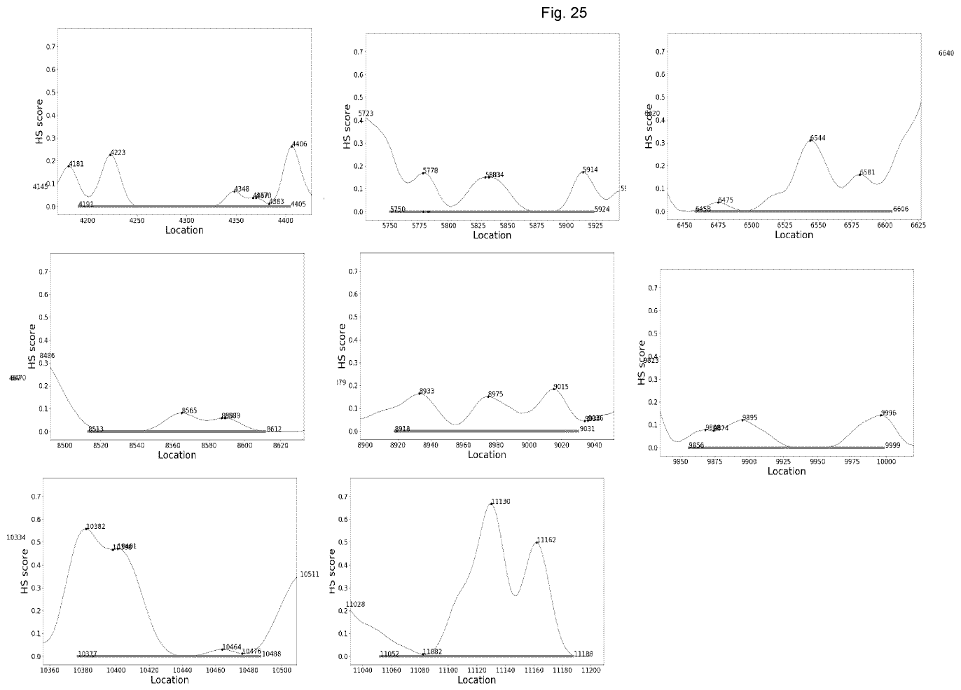

Step 2. After normalization, the scores from the five servers were summed, resulting in a sum S (x). S (x) is highly variable site to site, i.e. rugged, because each base pair is either being assigned a score or may be zero. In order to smooth the rugged distribution of S (x), a Gaussian filter with sigma=8 bp is applied, which gives a smoothed hotspot score HS (x). (FIG. 24 shows HS (x) for RACK1 post-splicing exonic mRNA, and FIG. 25 shows HS (x) for the 8 intron regions of pre-spliced RACK1 mRNA).

Step 3. The peaks of HS (x) indicate zones of the RNA sequence which are predicted to give effective siRNA prediction.

Known siRNA/shRNA are provided in Table 1 and their starting positions are labeled as plus sign in FIG. 24. The Santa Cruz siRNA is a mixture of three sequences that bind mRNA starting at position starting at 246, 631 and 892. The sequence shown in brackets in lower case “(aa)” is not a target sequence but an overhang sequence that can be incorporated when the antisense molecule is a siRNA.

TABLE 1
Known siRNA/shRNA

| mRNA | | SEQ |
|---|---|---|
| Starting | | ID |
| Position | Target Sequence | NO |
| 242 | ACCAGGGATGAGACCAACT^{[9]} | 70 |
| 246 | (aa)GGGATGAGACCAACTATGG ^{[7][8]} | 71 |
| 247 | GGATGAGACCAACTATGGAAT ^{[9]} | 72 |
| (shRNA) | | |
| 631 | (AA)GGTATGGAACCTGGCTAACG^{[8]} | 73 |
| 892 | (aa)GGGAAAGATCATTGTAGAT^{[7][8]} | 74 |
| 784 | (aa)CCTTTACACGCTAGATGGT^{[3]} | 75 |

Synthesized siRNA for RACK1 mRNA are provided in Table 2. Their corresponding peaks are labeled as star marker in FIG. 24.

TABLE 2
Synthesized siRNA for RACK1 mRNA

| | | | | SEQ |
|---|---|---|---|---|
| Peak | mRNA | | | ID |
| location | sequence | Target Sequence | | NO |
| 909 | 909-931 | (AT)GAACTGAA | | 2 |
| | (siR-2) | GCAAGAAGTTATC | | |
| 474 | 467-487 | (GT)CTCTGGAT | | 3 |
| | (siR-3) | CTCGAGATAAA | | |
| | | SEQ | | SEQ |
| Peak | antisense | ID | Sense | ID |
| location | (5′ to 3′) | NO | (5′ to 3′) | NO |
| 909 | GAUAACUUCUUG | 18 | GAACUGAAGC | 34 |
| | CUUCAGUUC(AU) | | AAGAAGUUAUC | |
| | | | (au) | |
| 474 | UUUAUCUCGAGA | 19 | CUCUGGAUCU | 35 |
| | UCCAGAG(gu) | | CGAGAUAAA(gu) | |

siRNA targeting mRNA: Within the coding region (sequence 108-1059), other significant peaks in FIG. 24 include positions 887, 909, 474, 212, 646, 618, 748, 779, 685, 242, 584, 295, 508, 988, 405, 160 and 178. Their corresponding targeting sequences are listed in Table 3. The sequences are listed in the order from higher HS (x) to lower HS (x).

TABLE 3
siRNA design for RACK1 mRNA

| | Target mRNA | Target mRNA | | SEQ ID |
|---|---|---|---|---|
| | sequence | Sequence | | NO of |
| | index | including | | target |
| Peak | including | overhang | | mRNA |
| location | overhang | brackets | | sequence |
| 887 | 884-904 | (GA)TTTAGAGGGAAAGATCATT | | 1 |
| 909 | 909-931 (siR-2) | (AT)GAACTGAAGCAAGAAGTTATC | | 2 |
| 474 | 467-487 (siR-3) | (GT)CTCTGGATCTCGAGATAAA | | 3 |
| 646 | 642-662 | (TG)GCTAACTGCAAGCTGAAGA | | 4 |
| 618 | 615-635 | (GG)GACAAGCTGGTCAAGGTAT | | 5 |
| 748 | 740-750 | (AA)GGATGGCCAGGCCATGTTA | | 6 |
| 779 | 779-799 | (AA)ACACCTTTACACGCTAGAT | | 7 |
| 685 | 683-703 | (GG)CTATCTGAACACGGTGACT | | 8 |
| 242 | 242-262 | (AC)CAGGGATGAGACCAACTAT | | 9 |
| 584 | 577-597 | (GC)CCAACAGCAGCAACCCTAT | | 10 |
| 295 | 296-316 | (CA)CTTTGTTAGTGATGTGGTT | | 11 |
| 508 | 505-525 | (TA)CCCTGGGTGTGTGCAAATA | | 12 |
| 988 | 981-1001 | (CT)GCTGATGGCCAGACTCTGT | | 13 |
| 405 | 403-423 | (GC)GATTTGTGGGCCATACCAA | | 14 |
| 160 | 156-176 | (GG)GTAACCCAGATCGCTACTA | | 15 |
| 178 | 178-198 | (CC)CGCAGTTCCCGGACATGAT | | 16 |
| | Antisense | | Sense(5′ to 3′) | |
| | (5′ to 3′) | | passenger | |
| | guide strand | SEQ | strand | SEQ |
| Peak | including | ID | including | ID |
| location | overhang | NO | overhang | NO |
| 887 | AAUGAUCUUUCCCUCUAAA(UC) | 17 | UUUAGAGGGAAAGAUCAUU(UC) | 33 |
| 909 | GAUAACUUCUUGCUUCAGUUC(AU) | 18 | GAACUGAAGCAAGAAGUUAUC(au) | 34 |
| (siR-2) | | | | |
| 474 | UUUAUCUCGAGAUCCAGAG(AC) | 19 | CUCUGGAUCUCGAGAUAAA(ac) | 35 |
| (siR-3) | | | | |
| 646 | UCUUCAGCUUGCAGUUAGC(CA) | 20 | GCUAACUGCAAGCUGAAGA(ca) | 36 |
| 618 | AUACCUUGACCAGCUUGUC(CC) | 21 | GACAAGCUGGUCAAGGUAU(CC) | 37 |
| 748 | UAACAUGGCCUGGCCAUCC(UU) | 22 | GGAUGGCCAGGCCAUGUUA(UU) | 38 |
| 779 | AUCUAGCGUGUAAAGGUGU(UU) | 23 | ACACCUUUACACGCUAGAU(UU) | 39 |
| 685 | AGUCACCGUGUUCAGAUAG(CC) | 24 | CUAUCUGAACACGGUGACU(CC) | 40 |
| 242 | AUAGUUGGUCUCAUCCCUG(GU) | 25 | CAGGGAUGAGACCAACUAU(gu) | 41 |
| 584 | AUAGGGUUGCUGCUGUUGG(GC) | 26 | CCAACAGCAGCAACCCUAU(gc) | 42 |
| 295 | AACCACAUCACUAACAAAG(UG) | 27 | CUUUGUUAGUGAUGUGGUU(ug) | 43 |
| 508 | UAUUUGCACACACCCAGGG(UA) | 28 | CCCUGGGUGUGUGCAAAUA(ua) | 44 |
| 988 | ACAGAGUCUGGCCAUCAGC(AG) | 29 | GCUGAUGGCCAGACUCUGU(ag) | 45 |
| 405 | UUGGUAUGGCCCACAAAUC(GC) | 30 | GAUUUGUGGGCCAUACCAA(gc) | 46 |
| 160 | UAGUAGCGAUCUGGGUUAC(CC) | 31 | GUAACCCAGAUCGCUACUA(CC) | 47 |
| 178 | AUCAUGUCCGGGAACUGCG(GG) | 32 | CGCAGUUCCCGGACAUGAU(gg) | 48 |

siRNA targeting pre-mRNA (Splice-blocking siRNA): Splice-blocking siRNA is designed to bind the boundary of intron and Extron region of RACK1 pre-mRNA. The hotspot score, HS (x), is constructed the same way as mRNA. The hotspot score of Extron-intron boundaries are extracted and shown in FIG. 25. The proposed target sequences are in Table 4. The sequence are listed from 5′ to 3′, or from N-terminal to C-terminal of the protein translation.

TABLE 4
siRNA targeting RACK1 pre-mRNA

| | Pre-mRNA | Target | | |
|---|---|---|---|---|
| | sequence | Sequence | | SEQ |
| Peak | including | including | | ID |
| location | overhang | overhang | | NO |
| 4406 | 4404-4424 | (AG)GTACGGACTA | | 49 |
| | | AGGTAGATT | | |
| −5750 | 5735-5755 | (TG)TTTTACCTCC | | 50 |
| | | TTTAGATAA | | |
| 10382 | 10366-10386 | (CC)TGTTCCCCAG | | 51 |
| | | GATTTAGAG | | |
| | Antisense | | Sense | |
| | (5′ to 3′) | | (5′ to 3′) | |
| | guide | | passenger | |
| | strand | SEQ | strand | SEQ |
| Peak | including | ID | including | ID |
| location | overhang | NO | overhang | NO |
| 4406 | AAUCUACCUUAG | 52 | GUACGGACUAA | 55 |
| | UCCGUAC(CU) | | GGUAGAUU(CU) | |
| −5750 | UUAUCUAAAGGA | 53 | UUUUACCUCCU | 56 |
| | GGUAAAA(CA) | | UUAGAUAA(ca) | |
| 10382 | CUCUAAAUCCUG | 54 | UGUUCCCCAGG | 57 |
| | GGGAACA(GG) | | AUUUAGAG(cg) | |

Negative control siRNA: To test the effectiveness of the prediction method, the low HS (x) score region was used as a negative control. The middle of each zero-score-region in FIG. 24 are listed in Table 5, in the order from wider to narrower zero-score-region in FIG. 24.

TABLE 5
Negative control of siRNA design
for RACK1 mRNA

| | Target Sequence | | | SEQ |
|---|---|---|---|---|
| mRNA | including | | | ID |
| sequence | overhang | | | NO |
| 100-120 | (CG)CCGCCATGACTGAGCAGAT | | | 58 |
| 362-382 | (AC)CCTGCGCCTCTGGGATCTC | | | 59 |
| 546-566 | (AC)TCAGAGTGGGTGTCTTGTG | | | 60 |
| 830-850 | (AG)CCCTAACCGCTACTGGCTG | | | 61 |
| | Antisense | | Sense | |
| | (5′ to 3′) | SEQ | (5′ to 3′) | SEQ |
| mRNA | including | ID | including | ID |
| sequence | overhang | NO | overhang | NO |
| 100-120 | AUCUGCUCAG | 62 | CCGCCAUGA | 66 |
| | UCAUGGCGG | | CUGAGCAGA | |
| | (CG) | | U(cg) | |
| 362-382 | GAGAUCCCAG | 63 | CCUGCGCCU | 67 |
| | AGGCGCAGG | | CUGGGAUCU | |
| | (GU) | | C(gu) | |
| 546-566 | CACAAGACAC | 64 | UCAGAGUGG | 68 |
| | CCACUCUGA | | GUGUCUUGU | |
| | (GU) | | G(gu) | |
| 830-850 | CAGCCAGUAG | 65 | CCCUAACCG | 69 |
| | CGGUUAGGG | | CUACUGGCU | |
| | (CU) | | G(cu) | |

### Results

siR-2 and siR-3 siRNA sequences successfully knocked down RACK1 (FIG. 26). HEK293T cells were seeded onto a 6-well plate (ThermoFisher Scientific) at a density of 250,000 cells per well the day prior to siRNA transfection. 10 μM stock of negative control or RACK1 siRNAs were introduced into the cell using Lipofectamine RNAiMAX transfection reagent (ThermoFisher Scientific) according to the manufacturer's instruction to achieve a final concentration of 25 pmol per well (or 1 pmol per 10,000 cells). 72 hrs post-transfection, cells were lysed in 2% SDS, followed by sonication at 30% power for 15 sec to extract total protein. Protein concentration was determined by BCA assay (ThermoFisher Scientific). 10 pg of protein from each sample was separated on 4-12% NuPAGE SDS-PAGE (ThermoFisher Scientific), transferred onto a PVDF membrane, and Western blotted for RACK1 and loading control α-tubulin as described above. Western blot band intensity was quantified using ImageJ. RACK1 intensity was normalized to corresponding α-tubulin intensity in each lane. Normalized RACK1 intensity of each transfection was then compared with un-transfected (UT) cells.

### Example 3: Knockdown of Rack1 Prevents hTDP-43-Induced Neurodegeneration In Vivo

As shown in Example 1 RACK1 knockdown in cultured cells ameliorates the phenotype caused by hTDP-43 expression in a number of ways, including by: reducing aggregation; restoring nuclear localization; and relieving TDP-43-induced suppression of protein synthesis. To extend these findings, it was further demonstrated herein that reduction of hTDP43-induced toxicity by RACK1 knockdown also takes place in vivo, in neurons functioning in a living network.

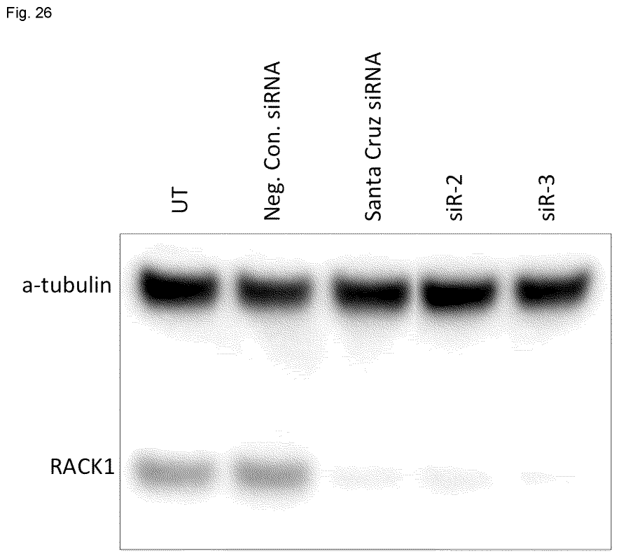

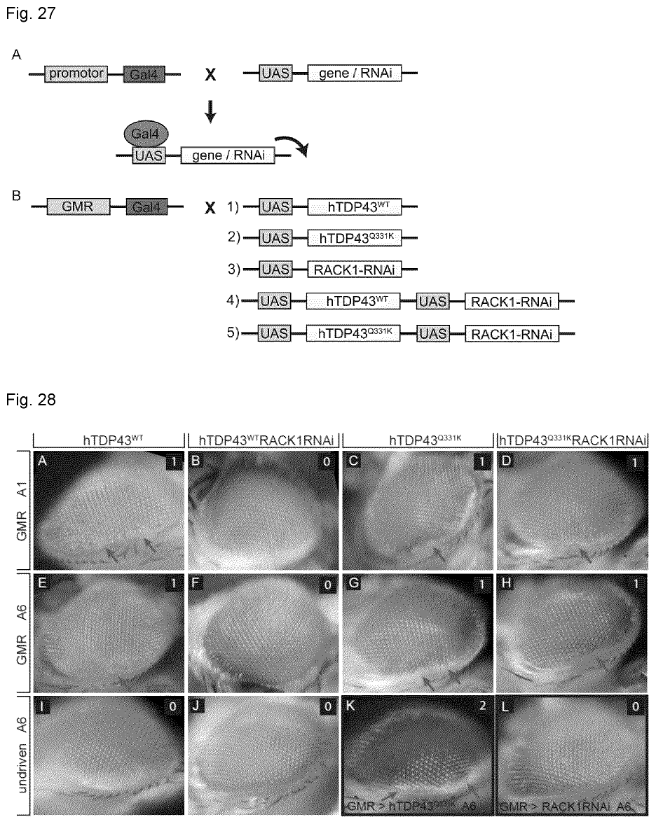

A Drosophila melanogaster expression system which allows modular, targeted expression was used. Using the UAS-Gal4 expression system (Rodriguez et al., 2012; explained in FIGS. 27A and 27B), expression of the alleles of interest was driven by the GMR promoter thus largely limiting expression to retinal neurons, a cell population widely used for its read-out of neuronal degeneration. Human TDP43 alleles wild-type (WT) and an ALS-associated point mutation (Q331K) (Elden et al. 2010) were used. Flies expressing hTDP43 either WT or Q331K, with or without RACK1-RNAi, in retinal neurons were generated (FIG. 27B).

With reference to FIG. 27A, in general, one line of flies harbors a transgene consisting of a promotor specific for the chosen cell population driving expression of the protein Gal4. A separate stable line of flies harbors a transgene with an upstream activating sequence (UAS) to drive expression of the sequence of interest, which may be protein-coding or RNAi. The UAS is not active, and these flies express no transgene. However, when these two lines of flies are crossed, producing offspring with one copy of each transgene, the F1 flies produce Gal4 protein only in the cells of interest, which then binds to and activates the UAS and turns on production of the gene/target of interest (Rodriguez et al., 2012). With reference to FIG. 27B, the GMR-Gal4 driver line (obtained from Bloomington Drosophila Stock Centre (BDSC) line #9146) which expresses Gal4 in retinal neurons, was used and crossed with one of five UAS lines:

1) UAS-hTDP43^{WT} (Elden et al., 2010; obtained from BDSC #79587)

2) UAS-hTDP43^{Q331K} (Elden et al., 2010; obtained from BDSC #79590)

3) UAS-RACK1-RNAi (Perkins et al., 2015; obtained from BDSC #60399)

4) 1 recombined onto the same chromosome with 3

5) 2 recombined onto the same chromosome with 3

These crosses produce flies expressing either wild-type or mutant hTDP43 or not, with or without RACK1-RNAi, in retinal neurons. Short hairpin RNA used to prepare the RNAi has Hairpin ID #SH047-D12; forward oligo is CAAGACCATCAAGCTGTGGAA (SEQ ID NO: 76), and reverse oligo is TTCCACAGCTTGATGGTCTTG (SEQ ID NO: 77). Since the parental lines are heterozygous for each transgene, having also a balancer chromosome with marker, siblings of the experimental flies are also produced which harbor only the driver or only the undriven UAS transgene. These flies are used as controls.

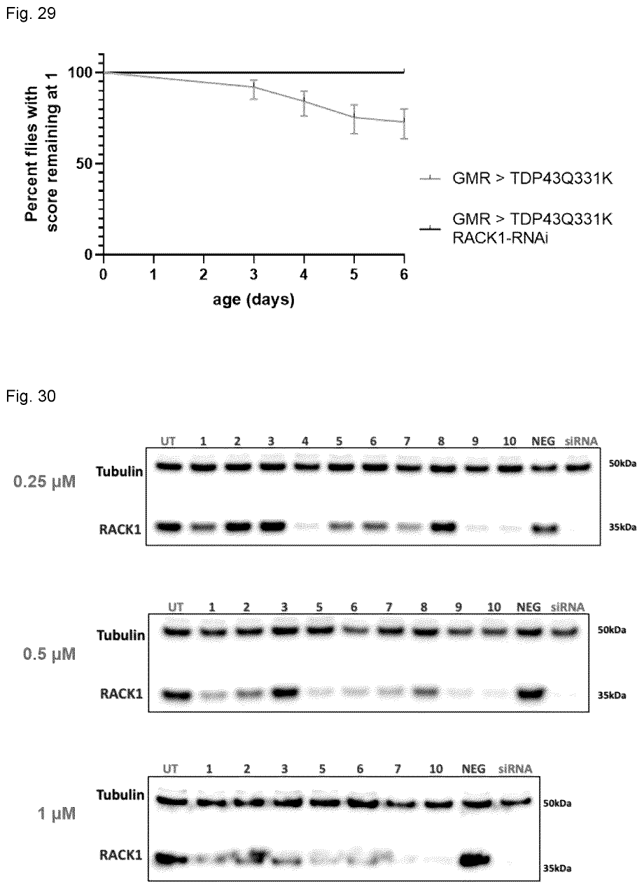

Cohorts of flies of each genotype were monitored for the first six days of adulthood (A1 to A6), and scored each day for retinal neuron degeneration. Control flies of a variety of genotypes provide a baseline for normal eye morphology. Representative photographs are provided in FIGS. 28A to 28L, and detailed numbers with statistical analysis are given in FIG. 29 and Tables 6 and 7 below. In eyes displaying mild degeneration, ommatidia are often missing from the ventral margin (arrows), in contrast to eyes without degeneration in which this margin is clearly intact. Additionally, darker dots of dying ommatidia can be observed.

As shown in FIGS. 28A, 28E, 28I (left column), hTDP43^{WT} causes mild neurodegeneration at A1 (FIG. 28A) which persists to A6 (FIG. 28E) and is absent in control (FIG. 28I). As shown in FIGS. 28B, 28F, 28J (second column), flies co-expressing of RACK1-RNAi with hTDP43^{WT} have no degeneration at A1 (FIG. 28B) or A6 (FIG. 28F), indistinguishable from control (FIG. 28J). As shown in FIGS. 28C, 28G, 28K (third column), hTDP43^{Q331K} causes degeneration which is mild at A1 (FIG. 28C), but worsens over time leading to some mild (FIG. 28G) and some moderate (FIG. 28K) cases at A6. When RACK1-RNAi is co-expressed with hTDP43^{Q331K} degeneration remains mild from A1 (FIG. 28D) to A6 (FIG. 28H). FIG. 28L is an additional control showing that GMR expression of RACK1-RNAi alone causes no phenotype. Flies were scored according to the system published by Li et al., 2010: 0=normal; 1=<25% ommatidia loss; 2=25-50% ommatidia loss; 3=50-75% ommatidia loss with small regions of necrosis (black patches); 4=>75% ommatidia loss with massive regions of necrosis. In each panel, the number at top right indicates the score which that eye received.

Quantification of retinal degeneration is shown in FIG. 29. Table 6 shows results of neurodegeneration scores for fly eyes.

TABLE 6
Neurodegeneration scores for fly eyes
PERCENT with each score

| | | age: | | | | | |
|---|---|---|---|---|---|---|---|
| genotype: | score: | A1 | A2 | A3 | A4 | A5 | A6 |
| GMR > hTDP43^{WT} | 0 | | | | | | |
| | 1 | 100 | 100 | 100 | 100 | 100 | 100 |
| | 2 | | | | | | |
| GMR > hTDP43^{WT} | 0 | 100 | 100 | 100 | 100 | 100 | 100 |
| RACK1-RNAi | 1 | | | | | | |
| | 2 | | | | | | |
| GMR > hTDP43^{Q331K} | 0 | | | | | | |
| | 1 | 100 | 100 | 92 | 84 | 75 | 72 |
| | 2 | | | 8 | 16 | 25 | 28 |
| GMR > hTDP43^{Q331K} | 0 | | | | | | |
| RACK1-RNAi | 1 | 100 | 100 | 100 | 100 | 100 | 100 |
| | 2 | | | | | | |
| GMR > RACK1-RNAi | 0 | 100 | 100 | 100 | 100 | 100 | 100 |
| GMR alone | 0 | 100 | 100 | 100 | 100 | 100 | 100 |
| hTDP43^{WT} (undriven) | 0 | 100 | 100 | 100 | 100 | 100 | 100 |
| hTDP43^{WT} RACK1-RNAi | 0 | 100 | 100 | 100 | 100 | 100 | 100 |
| (undriven) | | | | | | | |
| hTDP43^{Q331K} (undriven) | 0 | 100 | 100 | 100 | 100 | 100 | 100 |

Approximately 50 flies per genotype were scored each day. For the experimental flies, 3 rows indicate the percentage of flies which received a score of 0, 1 or 2 on each of days A1 to A6. 100% of GMR >hTDP43^{WT} scored 1 every day, while 100% of GMR >hTDP43^{WT} RACK1-RNAi scored 0 every day. GMR >hTDP43^{Q331K} flies all scored 1 on A1 and A2, but an increasing proportion worsened to score 2 on subsequent days. GMR >hTDP43^{Q331K} RACK1-RNAi all scored 1 at A1-A6. The various controls scored 0 at all ages. As shown in Table 7, Chi-squared tests were carried out as pair-wise comparisons, and extremely low p values show that all the indicated pairs of cohorts were significantly different from each other: hTDP43^{W}T is different from control (line 1); mutant TDP43 is different from WT (line 3); and the addition of RACK1-RNAi makes a significant difference to both hTDP43^{WT} (line 2) and hTDP43^{Q331K} (line 4). In FIG. 29, a Kaplan-Meier curve for GMR >hTDP43^{Q331K} (the only genotype which worsens with age) shows the percentage of flies whose score remains at 1 (rather than declining to 2) on any given day. This is shown in comparison to GMR >hTDP43^{Q331K} RACK1-RNAi, from which it is highly significantly different (Log-rank test: p=0.002. Error bars are 95% confidence intervals).

TABLE 7
Chi-squared tests

| genotype 1 | genotype 2 | p (Chi² test) |
|---|---|---|
| hTDP43^{WT} (undriven) | GMR > hTDP43^{WT} | 0.84 × 10⁻²⁸ |
| GMR > hTDP43^{WT} | GMR > hTDP43^{WT} | 0.19 × 10⁻²¹ |
| | RACK1-RNAi | |
| GMR > hTDP43^{WT} | GMR > hTDP43^{Q331K} (A6) | 0.30 × 10⁻⁴ |
| GMR > hTDP43^{Q331K} (A6) | GMR > hTDP43^{Q331K} | 0.0059 |
| | RACK1-RNAi | |

It was found that all flies expressing hTDP-43^{WT} in retinal neurons displayed mild neurodegeneration, replicating published findings (Elden et al., 2010). This was evident at A1 (FIG. 28A) and did not change over the next five days (FIG. 28E, and Tables 6 and 7, top rows). In striking contrast, 100% of flies co-expressing RACK1-RNAi with hTDP-43^{WT} displayed normal eye morphology with no degeneration, at all ages (FIG. 28B, 28F, Tables 6 and 7, second rows). Thus, RACK1 knockdown completely rescues hTDP-43^{WT}-induced degeneration. Expression of mutant hTDP-43^{Q331K} also caused retinal neuron degeneration in 100% of flies (FIG. 28C, 28G, 28K). This was more severe than that caused by hTDP-43^{WT} expression, and also significantly worsened over time (Tables 6 and 7, third rows, FIG. 29), thus modeling two features of disease. In contrast, flies co-expressing RACK1-RNAi with hTDP-43^{Q331K} displayed mild degeneration that remained mild from A1 to A6 (FIGS. 2D, 2H, Tables 6 and 7, fourth rows, FIG. 29). Thus, RACK1 knockdown completely prevents the worsening of neurodegeneration overtime caused by hTDP-43^{Q331K}

### Example 4: Antisense Oligonucleotides

Antisense oligonucleotides (ASOs) binding human RACK1 mRNA were generated and are detailed in Table 8. Modifications to the bases are as follows. The ASOs have phosphorothioate bonds between all bases. The 2′-O-methoxyethyl(2′-MOE) modification is used for the 5 RNA bases on each end, with 10 DNA bases in the middle to form a ‘gapmer’ structure. The mRNA start position at which the ASO sequences bind RACK1 are indicated. Although the ASO sequences may be represented as DNA, RNA where thymidine (T) is uracil (U) also contemplated.

TABLE 8
ASO sequences

| | SEQ | Target | Target | Corresponding mRNA | SEQ |
|---|---|---|---|---|---|
| | ID | mRNA | mRNA | (reverse complement | ID |
| ASO sequence | NO | start | end | of ASO) | NO |
| TGGTCTCATCCCTGGTCAGT | 78 | 238 | 257 | ACTGACCAGGGATGAGACCA | 289 |
| [ASO #1] | | | | | |
| CACGAAGGGTCATCTGCTCA | 79 | 112 | 131 | TGAGCAGATGACCCTTCGTG | 290 |
| [ASO #2] | | | | | |
| CAGGTTCCATACCTTGACCA | 80 | 624 | 643 | TGGTCAAGGTATGGAACCTG | 291 |
| [ASO #3] | | | | | |
| TCCAGAGACAATCTGCCGGT | 81 | 456 | 475 | ACCGGCAGATTGTCTCTGGA | 292 |
| [ASO #4] | | | | | |
| ACCGTGTTCAGATAGCCTGT | 82 | 680 | 699 | ACAGGCTATCTGAACACGGT | 293 |
| [ASO #5] | | | | | |
| ATCATGTCCGGGAACTGCGG | 83 | 179 | 198 | CCGCAGTTCCCGGACATGAT | 294 |
| [ASO #6] | | | | | |
| CGTTGTGAGATCCCAGAGGC | 84 | 369 | 388 | GCCTCTGGGATCTCACAACG | 295 |
| [ASO #7] | | | | | |
| GCCGGTTGTCAGAGGAGAAG | 85 | 442 | 461 | CTTCTCCTCTGACAACCGGC | 296 |
| [ASO #8] | | | | | |
| CCAGAGACAATCTGCCGGTT | 86 | 455 | 474 | AACCGGCAGATTGTCTCTGG | 297 |
| [ASO #9] | | | | | |
| ACGATGATAGGGTTGCTGCT | 87 | 584 | 603 | AGCAGCAACCCTATCATCGT | 298 |
| [ASO #10] | | | | | |
| AAGGGTCATCTGCTCAGTCA | 88 | 108 | 127 | TGACTGAGCAGATGACCCTT | 299 |
| GAAGGGTCATCTGCTCAGTC | 89 | 109 | 128 | GACTGAGCAGATGACCCTTC | 300 |
| CGAAGGGTCATCTGCTCAGT | 90 | 110 | 129 | ACTGAGCAGATGACCCTTCG | 301 |
| ACGAAGGGTCATCTGCTCAG | 91 | 111 | 130 | CTGAGCAGATGACCCTTCGT | 302 |
| CCACGAAGGGTCATCTGCTC | 92 | 113 | 132 | GAGCAGATGACCCTTCGTGG | 303 |
| GCCACGAAGGGTCATCTGCT | 93 | 114 | 133 | AGCAGATGACCCTTCGTGGC | 304 |
| TGCCACGAAGGGTCATCTGC | 94 | 115 | 134 | GCAGATGACCCTTCGTGGCA | 305 |
| GTGCCACGAAGGGTCATCTG | 95 | 116 | 135 | CAGATGACCCTTCGTGGCAC | 306 |
| GGTGCCACGAAGGGTCATCT | 96 | 117 | 136 | AGATGACCCTTCGTGGCACC | 307 |
| GGGTGCCACGAAGGGTCATC | 97 | 118 | 137 | GATGACCCTTCGTGGCACCC | 308 |
| AGGGTGCCACGAAGGGTCAT | 98 | 119 | 138 | ATGACCCTTCGTGGCACCCT | 309 |
| GAGGGTGCCACGAAGGGTCA | 99 | 120 | 139 | TGACCCTTCGTGGCACCCTC | 310 |
| TGAGGGTGCCACGAAGGGTC | 100 | 121 | 140 | GACCCTTCGTGGCACCCTCA | 311 |
| TTGAGGGTGCCACGAAGGGT | 101 | 122 | 141 | ACCCTTCGTGGCACCCTCAA | 312 |
| CTTGAGGGTGCCACGAAGGG | 102 | 123 | 142 | CCCTTCGTGGCACCCTCAAG | 313 |
| CCTTGAGGGTGCCACGAAGG | 103 | 124 | 143 | CCTTCGTGGCACCCTCAAGG | 314 |
| CCCTTGAGGGTGCCACGAAG | 104 | 125 | 144 | CTTCGTGGCACCCTCAAGGG | 315 |
| GCCCTTGAGGGTGCCACGAA | 105 | 126 | 145 | TTCGTGGCACCCTCAAGGGC | 316 |
| GGCCCTTGAGGGTGCCACGA | 106 | 127 | 146 | TCGTGGCACCCTCAAGGGCC | 317 |
| TGCGGGGTAGTAGCGATCTG | 107 | 164 | 183 | CAGATCGCTACTACCCCGCA | 318 |
| CTGCGGGGTAGTAGCGATCT | 108 | 165 | 184 | AGATCGCTACTACCCCGCAG | 319 |
| ACTGCGGGGTAGTAGCGATC | 109 | 166 | 185 | GATCGCTACTACCCCGCAGT | 320 |
| AACTGCGGGGTAGTAGCGAT | 110 | 167 | 186 | ATCGCTACTACCCCGCAGTT | 321 |
| GAACTGCGGGGTAGTAGCGA | 111 | 168 | 187 | TCGCTACTACCCCGCAGTTC | 322 |
| GGAACTGCGGGGTAGTAGCG | 112 | 169 | 188 | CGCTACTACCCCGCAGTTCC | 323 |
| GGGAACTGCGGGGTAGTAGC | 113 | 170 | 189 | GCTACTACCCCGCAGTTCCC | 324 |
| CGGGAACTGCGGGGTAGTAG | 114 | 171 | 190 | CTACTACCCCGCAGTTCCCG | 325 |
| CCGGGAACTGCGGGGTAGTA | 115 | 172 | 191 | TACTACCCCGCAGTTCCCGG | 326 |
| TCCGGGAACTGCGGGGTAGT | 116 | 173 | 192 | ACTACCCCGCAGTTCCCGGA | 327 |
| GTCCGGGAACTGCGGGGTAG | 117 | 174 | 193 | CTACCCCGCAGTTCCCGGAC | 328 |
| TGTCCGGGAACTGCGGGGTA | 118 | 175 | 194 | TACCCCGCAGTTCCCGGACA | 329 |
| ATGTCCGGGAACTGCGGGGT | 119 | 176 | 195 | ACCCCGCAGTTCCCGGACAT | 330 |
| CATGTCCGGGAACTGCGGGG | 120 | 177 | 196 | CCCCGCAGTTCCCGGACATG | 331 |
| TCATGTCCGGGAACTGCGGG | 121 | 178 | 197 | CCCGCAGTTCCCGGACATGA | 332 |
| GATCATGTCCGGGAACTGCG | 122 | 180 | 199 | CGCAGTTCCCGGACATGATC | 333 |
| GGATCATGTCCGGGAACTGC | 123 | 181 | 200 | GCAGTTCCCGGACATGATCC | 334 |
| AGGATCATGTCCGGGAACTG | 124 | 182 | 201 | CAGTTCCCGGACATGATCCT | 335 |
| GAGGATCATGTCCGGGAACT | 125 | 183 | 202 | AGTTCCCGGACATGATCCTC | 336 |
| AGAGGATCATGTCCGGGAAC | 126 | 184 | 203 | GTTCCCGGACATGATCCTCT | 337 |
| GAGAGGATCATGTCCGGGAA | 127 | 185 | 204 | TTCCCGGACATGATCCTCTC | 338 |
| GGAGAGGATCATGTCCGGGA | 128 | 186 | 205 | TCCCGGACATGATCCTCTCC | 339 |
| CGGAGAGGATCATGTCCGGG | 129 | 187 | 206 | CCCGGACATGATCCTCTCCG | 340 |
| GCGGAGAGGATCATGTCCGG | 130 | 188 | 207 | CCGGACATGATCCTCTCCGC | 341 |
| GGCGGAGAGGATCATGTCCG | 131 | 189 | 208 | CGGACATGATCCTCTCCGCC | 342 |
| AGGCGGAGAGGATCATGTCC | 132 | 190 | 209 | GGACATGATCCTCTCCGCCT | 343 |
| GAGGCGGAGAGGATCATGTC | 133 | 191 | 210 | GACATGATCCTCTCCGCCTC | 344 |
| AGAGGCGGAGAGGATCATGT | 134 | 192 | 211 | ACATGATCCTCTCCGCCTCT | 345 |
| GAGAGGCGGAGAGGATCATG | 135 | 193 | 212 | CATGATCCTCTCCGCCTCTC | 346 |
| CGAGAGGCGGAGAGGATCAT | 136 | 194 | 213 | ATGATCCTCTCCGCCTCTCG | 347 |
| TCAGTTTCCACATGATGATG | 137 | 223 | 242 | CATCATCATGTGGAAACTGA | 348 |
| GTCAGTTTCCACATGATGAT | 138 | 224 | 243 | ATCATCATGTGGAAACTGAC | 349 |
| GGTCAGTTTCCACATGATGA | 139 | 225 | 244 | TCATCATGTGGAAACTGACC | 350 |
| TGGTCAGTTTCCACATGATG | 140 | 226 | 245 | CATCATGTGGAAACTGACCA | 351 |
| CTGGTCAGTTTCCACATGAT | 141 | 227 | 246 | ATCATGTGGAAACTGACCAG | 352 |
| CCTGGTCAGTTTCCACATGA | 142 | 228 | 247 | TCATGTGGAAACTGACCAGG | 353 |
| CCCTGGTCAGTTTCCACATG | 143 | 229 | 248 | CATGTGGAAACTGACCAGGG | 354 |
| TCCCTGGTCAGTTTCCACAT | 144 | 230 | 249 | ATGTGGAAACTGACCAGGGA | 355 |
| ATCCCTGGTCAGTTTCCACA | 145 | 231 | 250 | TGTGGAAACTGACCAGGGAT | 356 |
| CATCCCTGGTCAGTTTCCAC | 146 | 232 | 251 | GTGGAAACTGACCAGGGATG | 357 |
| TCATCCCTGGTCAGTTTCCA | 147 | 233 | 252 | TGGAAACTGACCAGGGATGA | 358 |
| CTCATCCCTGGTCAGTTTCC | 148 | 234 | 253 | GGAAACTGACCAGGGATGAG | 359 |
| TCTCATCCCTGGTCAGTTTC | 149 | 235 | 254 | GAAACTGACCAGGGATGAGA | 360 |
| GAGGCGCAGGGTTCCATCCC | 150 | 354 | 373 | GGGATGGAACCCTGCGCCTC | 361 |
| AGAGGCGCAGGGTTCCATCC | 151 | 355 | 374 | GGATGGAACCCTGCGCCTCT | 362 |
| CAGAGGCGCAGGGTTCCATC | 152 | 356 | 375 | GATGGAACCCTGCGCCTCTG | 363 |
| CCAGAGGCGCAGGGTTCCAT | 153 | 357 | 376 | ATGGAACCCTGCGCCTCTGG | 364 |
| CCGTTGTGAGATCCCAGAGG | 154 | 370 | 389 | CCTCTGGGATCTCACAACGG | 365 |
| CCCGTTGTGAGATCCCAGAG | 155 | 371 | 390 | CTCTGGGATCTCACAACGGG | 366 |
| GCCCGTTGTGAGATCCCAGA | 156 | 372 | 391 | TCTGGGATCTCACAACGGGC | 367 |
| TGCCCGTTGTGAGATCCCAG | 157 | 373 | 392 | CTGGGATCTCACAACGGGCA | 368 |
| GTGCCCGTTGTGAGATCCCA | 158 | 374 | 393 | TGGGATCTCACAACGGGCAC | 369 |
| GGTGCCCGTTGTGAGATCCC | 159 | 375 | 394 | GGGATCTCACAACGGGCACC | 370 |
| TGGTGCCCGTTGTGAGATCC | 160 | 376 | 395 | GGATCTCACAACGGGCACCA | 371 |
| GTGGTGCCCGTTGTGAGATC | 161 | 377 | 396 | GATCTCACAACGGGCACCAC | 372 |
| GGTGGTGCCCGTTGTGAGAT | 162 | 378 | 397 | ATCTCACAACGGGCACCACC | 373 |
| TGGTGGTGCCCGTTGTGAGA | 163 | 379 | 398 | TCTCACAACGGGCACCACCA | 374 |
| GTGGTGGTGCCCGTTGTGAG | 164 | 380 | 399 | CTCACAACGGGCACCACCAC | 375 |
| CGTGGTGGTGCCCGTTGTGA | 165 | 381 | 400 | TCACAACGGGCACCACCACG | 376 |
| TCGTGGTGGTGCCCGTTGTG | 166 | 382 | 401 | CACAACGGGCACCACCACGA | 377 |
| CTCGTGGTGGTGCCCGTTGT | 167 | 383 | 402 | ACAACGGGCACCACCACGAG | 378 |
| CCTCGTGGTGGTGCCCGTTG | 168 | 384 | 403 | CAACGGGCACCACCACGAGG | 379 |
| AGAAGGCCACACTCAGCACA | 169 | 427 | 446 | TGTGCTGAGTGTGGCCTTCT | 380 |
| GAGAAGGCCACACTCAGCAC | 170 | 428 | 447 | GTGCTGAGTGTGGCCTTCTC | 381 |
| GGAGAAGGCCACACTCAGCA | 171 | 429 | 448 | TGCTGAGTGTGGCCTTCTCC | 382 |
| AGGAGAAGGCCACACTCAGC | 172 | 430 | 449 | GCTGAGTGTGGCCTTCTCCT | 383 |
| GAGGAGAAGGCCACACTCAG | 173 | 431 | 450 | CTGAGTGTGGCCTTCTCCTC | 384 |
| AGAGGAGAAGGCCACACTCA | 174 | 432 | 451 | TGAGTGTGGCCTTCTCCTCT | 385 |
| CAGAGGAGAAGGCCACACTC | 175 | 433 | 452 | GAGTGTGGCCTTCTCCTCTG | 386 |
| TCAGAGGAGAAGGCCACACT | 176 | 434 | 453 | AGTGTGGCCTTCTCCTCTGA | 387 |
| GTCAGAGGAGAAGGCCACAC | 177 | 435 | 454 | GTGTGGCCTTCTCCTCTGAC | 388 |
| TGTCAGAGGAGAAGGCCACA | 178 | 436 | 455 | TGTGGCCTTCTCCTCTGACA | 389 |
| TTGTCAGAGGAGAAGGCCAC | 179 | 437 | 456 | GTGGCCTTCTCCTCTGACAA | 390 |
| GTTGTCAGAGGAGAAGGCCA | 180 | 438 | 457 | TGGCCTTCTCCTCTGACAAC | 391 |
| GGTTGTCAGAGGAGAAGGCC | 181 | 439 | 458 | GGCCTTCTCCTCTGACAACC | 392 |
| CGGTTGTCAGAGGAGAAGGC | 182 | 440 | 459 | GCCTTCTCCTCTGACAACCG | 393 |
| CCGGTTGTCAGAGGAGAAGG | 183 | 441 | 460 | CCTTCTCCTCTGACAACCGG | 394 |
| TGCCGGTTGTCAGAGGAGAA | 184 | 443 | 462 | TTCTCCTCTGACAACCGGCA | 395 |
| CTGCCGGTTGTCAGAGGAGA | 185 | 444 | 463 | TCTCCTCTGACAACCGGCAG | 396 |
| TCTGCCGGTTGTCAGAGGAG | 186 | 445 | 464 | CTCCTCTGACAACCGGCAGA | 397 |
| ATCTGCCGGTTGTCAGAGGA | 187 | 446 | 465 | TCCTCTGACAACCGGCAGAT | 398 |
| AATCTGCCGGTTGTCAGAGG | 188 | 447 | 466 | CCTCTGACAACCGGCAGATT | 399 |
| CAATCTGCCGGTTGTCAGAG | 189 | 448 | 467 | CTCTGACAACCGGCAGATTG | 400 |
| ACAATCTGCCGGTTGTCAGA | 190 | 449 | 468 | TCTGACAACCGGCAGATTGT | 401 |
| GACAATCTGCCGGTTGTCAG | 191 | 450 | 469 | CTGACAACCGGCAGATTGTC | 402 |
| AGACAATCTGCCGGTTGTCA | 192 | 451 | 470 | TGACAACCGGCAGATTGTCT | 403 |
| GAGACAATCTGCCGGTTGTC | 193 | 452 | 471 | GACAACCGGCAGATTGTCTC | 404 |
| AGAGACAATCTGCCGGTTGT | 194 | 453 | 472 | ACAACCGGCAGATTGTCTCT | 405 |
| CAGAGACAATCTGCCGGTTG | 195 | 454 | 473 | CAACCGGCAGATTGTCTCTG | 406 |
| ATCCAGAGACAATCTGCCGG | 196 | 457 | 476 | CCGGCAGATTGTCTCTGGAT | 407 |
| GATCCAGAGACAATCTGCCG | 197 | 458 | 477 | CGGCAGATTGTCTCTGGATC | 408 |
| AGATCCAGAGACAATCTGCC | 198 | 459 | 478 | GGCAGATTGTCTCTGGATCT | 409 |
| GAGATCCAGAGACAATCTGC | 199 | 460 | 479 | GCAGATTGTCTCTGGATCTC | 410 |
| CGAGATCCAGAGACAATCTG | 200 | 461 | 480 | CAGATTGTCTCTGGATCTCG | 411 |
| TCGAGATCCAGAGACAATCT | 201 | 462 | 481 | AGATTGTCTCTGGATCTCGA | 412 |
| CTCGAGATCCAGAGACAATC | 202 | 463 | 482 | GATTGTCTCTGGATCTCGAG | 413 |
| TCTCGAGATCCAGAGACAAT | 203 | 464 | 483 | ATTGTCTCTGGATCTCGAGA | 414 |
| ATCTCGAGATCCAGAGACAA | 204 | 465 | 484 | TTGTCTCTGGATCTCGAGAT | 415 |
| TATCTCGAGATCCAGAGACA | 205 | 466 | 485 | TGTCTCTGGATCTCGAGATA | 416 |
| TTATCTCGAGATCCAGAGAC | 206 | 467 | 486 | GTCTCTGGATCTCGAGATAA | 417 |
| TTTATCTCGAGATCCAGAGA | 207 | 468 | 487 | TCTCTGGATCTCGAGATAAA | 418 |
| TTTTATCTCGAGATCCAGAG | 208 | 469 | 488 | CTCTGGATCTCGAGATAAAA | 419 |
| GTTTTATCTCGAGATCCAGA | 209 | 470 | 489 | TCTGGATCTCGAGATAAAAC | 420 |
| GGTTTTATCTCGAGATCCAG | 210 | 471 | 490 | CTGGATCTCGAGATAAAACC | 421 |
| CTGCTGTTGGGCGAGAAGCG | 211 | 569 | 588 | CGCTTCTCGCCCAACAGCAG | 422 |
| GCTGCTGTTGGGCGAGAAGC | 212 | 570 | 589 | GCTTCTCGCCCAACAGCAGC | 423 |
| TGCTGCTGTTGGGCGAGAAG | 213 | 571 | 590 | CTTCTCGCCCAACAGCAGCA | 424 |
| TTGCTGCTGTTGGGCGAGAA | 214 | 572 | 591 | TTCTCGCCCAACAGCAGCAA | 425 |
| GTTGCTGCTGTTGGGCGAGA | 215 | 573 | 592 | TCTCGCCCAACAGCAGCAAC | 426 |
| GGTTGCTGCTGTTGGGCGAG | 216 | 574 | 593 | CTCGCCCAACAGCAGCAACC | 427 |
| GGGTTGCTGCTGTTGGGCGA | 217 | 575 | 594 | TCGCCCAACAGCAGCAACCC | 428 |
| AGGGTTGCTGCTGTTGGGCG | 218 | 576 | 595 | CGCCCAACAGCAGCAACCCT | 429 |
| TAGGGTTGCTGCTGTTGGGC | 219 | 577 | 596 | GCCCAACAGCAGCAACCCTA | 430 |
| ATAGGGTTGCTGCTGTTGGG | 220 | 578 | 597 | CCCAACAGCAGCAACCCTAT | 431 |
| GATAGGGTTGCTGCTGTTGG | 221 | 579 | 598 | CCAACAGCAGCAACCCTATC | 432 |
| TGATAGGGTTGCTGCTGTTG | 222 | 580 | 599 | CAACAGCAGCAACCCTATCA | 433 |
| ATGATAGGGTTGCTGCTGTT | 223 | 581 | 600 | AACAGCAGCAACCCTATCAT | 434 |
| GATGATAGGGTTGCTGCTGT | 224 | 582 | 601 | ACAGCAGCAACCCTATCATC | 435 |
| CGATGATAGGGTTGCTGCTG | 225 | 583 | 602 | CAGCAGCAACCCTATCATCG | 436 |
| GACGATGATAGGGTTGCTGC | 226 | 585 | 604 | GCAGCAACCCTATCATCGTC | 437 |
| AGACGATGATAGGGTTGCTG | 227 | 586 | 605 | CAGCAACCCTATCATCGTCT | 438 |
| GAGACGATGATAGGGTTGCT | 228 | 587 | 606 | AGCAACCCTATCATCGTCTC | 439 |
| GGAGACGATGATAGGGTTGC | 229 | 588 | 607 | GCAACCCTATCATCGTCTCC | 440 |
| AGGAGACGATGATAGGGTTG | 230 | 589 | 608 | CAACCCTATCATCGTCTCCT | 441 |
| CAGGAGACGATGATAGGGTT | 231 | 590 | 609 | AACCCTATCATCGTCTCCTG | 442 |
| ACAGGAGACGATGATAGGGT | 232 | 591 | 610 | ACCCTATCATCGTCTCCTGT | 443 |
| CACAGGAGACGATGATAGGG | 233 | 592 | 611 | CCCTATCATCGTCTCCTGTG | 444 |
| CCACAGGAGACGATGATAGG | 234 | 593 | 612 | CCTATCATCGTCTCCTGTGG | 445 |
| GCCACAGGAGACGATGATAG | 235 | 594 | 613 | CTATCATCGTCTCCTGTGGC | 446 |
| AGCCACAGGAGACGATGATA | 236 | 595 | 614 | TATCATCGTCTCCTGTGGCT | 447 |
| CAGCCACAGGAGACGATGAT | 237 | 596 | 615 | ATCATCGTCTCCTGTGGCTG | 448 |
| CCAGCCACAGGAGACGATGA | 238 | 597 | 616 | TCATCGTCTCCTGTGGCTGG | 449 |
| CCCAGCCACAGGAGACGATG | 239 | 598 | 617 | CATCGTCTCCTGTGGCTGGG | 450 |
| TCCCAGCCACAGGAGACGAT | 240 | 599 | 618 | ATCGTCTCCTGTGGCTGGGA | 451 |
| GACCAGCTTGTCCCAGCCAC | 241 | 609 | 628 | GTGGCTGGGACAAGCTGGTC | 452 |
| TGACCAGCTTGTCCCAGCCA | 242 | 610 | 629 | TGGCTGGGACAAGCTGGTCA | 453 |
| TTGACCAGCTTGTCCCAGCC | 243 | 611 | 630 | GGCTGGGACAAGCTGGTCAA | 454 |
| CTTGACCAGCTTGTCCCAGC | 244 | 612 | 631 | GCTGGGACAAGCTGGTCAAG | 455 |
| CCTTGACCAGCTTGTCCCAG | 245 | 613 | 632 | CTGGGACAAGCTGGTCAAGG | 456 |
| ACCTTGACCAGCTTGTCCCA | 246 | 614 | 633 | TGGGACAAGCTGGTCAAGGT | 457 |
| TACCTTGACCAGCTTGTCCC | 247 | 615 | 634 | GGGACAAGCTGGTCAAGGTA | 458 |
| ATACCTTGACCAGCTTGTCC | 248 | 616 | 635 | GGACAAGCTGGTCAAGGTAT | 459 |
| CATACCTTGACCAGCTTGTC | 249 | 617 | 636 | GACAAGCTGGTCAAGGTATG | 460 |
| CCATACCTTGACCAGCTTGT | 250 | 618 | 637 | ACAAGCTGGTCAAGGTATGG | 461 |
| TCCATACCTTGACCAGCTTG | 251 | 619 | 638 | CAAGCTGGTCAAGGTATGGA | 462 |
| TTCCATACCTTGACCAGCTT | 252 | 620 | 639 | AAGCTGGTCAAGGTATGGAA | 463 |
| GTTCCATACCTTGACCAGCT | 253 | 621 | 640 | AGCTGGTCAAGGTATGGAAC | 464 |
| GGTTCCATACCTTGACCAGC | 254 | 622 | 641 | GCTGGTCAAGGTATGGAACC | 465 |
| AGGTTCCATACCTTGACCAG | 255 | 623 | 642 | CTGGTCAAGGTATGGAACCT | 466 |
| GCTTGCAGTTAGCCAGGTTC | 256 | 637 | 656 | GAACCTGGCTAACTGCAAGC | 467 |
| AGCTTGCAGTTAGCCAGGTT | 257 | 638 | 657 | AACCTGGCTAACTGCAAGCT | 468 |
| CAGCTTGCAGTTAGCCAGGT | 258 | 639 | 658 | ACCTGGCTAACTGCAAGCTG | 469 |
| CCTGTGTGGCCAATGTGGTT | 259 | 665 | 684 | AACCACATTGGCCACACAGG | 470 |
| GCCTGTGTGGCCAATGTGGT | 260 | 666 | 685 | ACCACATTGGCCACACAGGC | 471 |
| AGCCTGTGTGGCCAATGTGG | 261 | 667 | 686 | CCACATTGGCCACACAGGCT | 472 |
| TAGCCTGTGTGGCCAATGTG | 262 | 668 | 687 | CACATTGGCCACACAGGCTA | 473 |
| ATAGCCTGTGTGGCCAATGT | 263 | 669 | 688 | ACATTGGCCACACAGGCTAT | 474 |
| GATAGCCTGTGTGGCCAATG | 264 | 670 | 689 | CATTGGCCACACAGGCTATC | 475 |
| AGATAGCCTGTGTGGCCAAT | 265 | 671 | 690 | ATTGGCCACACAGGCTATCT | 476 |
| CAGATAGCCTGTGTGGCCAA | 266 | 672 | 691 | TTGGCCACACAGGCTATCTG | 477 |
| TCAGATAGCCTGTGTGGCCA | 267 | 673 | 692 | TGGCCACACAGGCTATCTGA | 478 |
| TTCAGATAGCCTGTGTGGCC | 268 | 674 | 693 | GGCCACACAGGCTATCTGAA | 479 |
| GTTCAGATAGCCTGTGTGGC | 269 | 675 | 694 | GCCACACAGGCTATCTGAAC | 480 |
| TGTTCAGATAGCCTGTGTGG | 270 | 676 | 695 | CCACACAGGCTATCTGAACA | 481 |
| GTGTTCAGATAGCCTGTGTG | 271 | 677 | 696 | CACACAGGCTATCTGAACAC | 482 |
| CGTGTTCAGATAGCCTGTGT | 272 | 678 | 697 | ACACAGGCTATCTGAACACG | 483 |
| CCGTGTTCAGATAGCCTGTG | 273 | 679 | 698 | CACAGGCTATCTGAACACGG | 484 |
| CACCGTGTTCAGATAGCCTG | 274 | 681 | 700 | CAGGCTATCTGAACACGGTG | 485 |
| TCACCGTGTTCAGATAGCCT | 275 | 682 | 701 | AGGCTATCTGAACACGGTGA | 486 |
| GTCACCGTGTTCAGATAGCC | 276 | 683 | 702 | GGCTATCTGAACACGGTGAC | 487 |
| AGTCACCGTGTTCAGATAGC | 277 | 684 | 703 | GCTATCTGAACACGGTGACT | 488 |
| CAGTCACCGTGTTCAGATAG | 278 | 685 | 704 | CTATCTGAACACGGTGACTG | 489 |
| ACAGTCACCGTGTTCAGATA | 279 | 686 | 705 | TATCTGAACACGGTGACTGT | 490 |
| GACAGTCACCGTGTTCAGAT | 280 | 687 | 706 | ATCTGAACACGGTGACTGTC | 491 |
| AGACAGTCACCGTGTTCAGA | 281 | 688 | 707 | TCTGAACACGGTGACTGTCT | 492 |
| GAGACAGTCACCGTGTTCAG | 282 | 689 | 708 | CTGAACACGGTGACTGTCTC | 493 |
| AGAGACAGTCACCGTGTTCA | 283 | 690 | 709 | TGAACACGGTGACTGTCTCT | 494 |
| GAGAGACAGTCACCGTGTTC | 284 | 691 | 710 | GAACACGGTGACTGTCTCTC | 495 |
| GGAGAGACAGTCACCGTGTT | 285 | 692 | 711 | AACACGGTGACTGTCTCTCC | 496 |
| TGGAGAGACAGTCACCGTGT | 286 | 693 | 712 | ACACGGTGACTGTCTCTCCA | 497 |
| CTGGAGAGACAGTCACCGTG | 287 | 694 | 713 | CACGGTGACTGTCTCTCCAG | 498 |
| TCTGGAGAGACAGTCACCGT | 288 | 695 | 714 | ACGGTGACTGTCTCTCCAGA | 499 |

### Example 5: In Vitro and In Vivo Study of Antisense Oligonucleotides

### Cell Culture

ASOs #1 to #10 described in Example 4 were tested in human-derived wild-type HeLa cells. Cells were cultured in Dulbecco's modified Eagle medium (DMEM) supplemented with 10% fetal bovine serum, GlutaMax™-1 (2 mM), penicillin (50 U/ml), and streptomycin (50 mg/ml) at 37° C. in 5% CO₂ One day before ASO treatment, naïve cells were seeded in 12-well plates (Corning™ Costar™ Flat Bottom Cell Culture Plates, ThermoFisher Scientific) at a density of 75,000 cells/well in 1 ml media and grown overnight to reach 20-30% confluency.

### ASO Treatment

250 nM, 500 nM, or 1 μM of ASOs #1 to #10 described in Example 4 were introduced into the cells using Lipofectamine RNAiMAX Transfection Reagent (ThermoFisher Scientific) at a ratio of 5 μl RNAiMAX per 1 μM ASO. Cells were incubated with fresh media containing ASO/RNAiMAX complexes for 72 hr until lysed.

### Protein Extraction and Immunoblotting

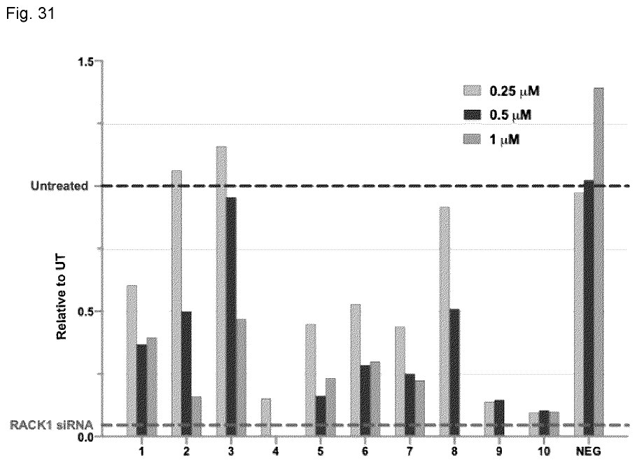

Cells were washed twice with ice-cold PBS, lysed in 2% SDS, and sonicated at 25% amplitude for 10 sec. Lysates were clarified by centrifugation at 14,000 RPM for 10 min and protein concentrations were determined by BCA (ThermoFisher Scientific). 3-5 ug* of each sample was separated by on 4-12% NuPAGE Bis-Tris SDS-PAGE (ThermoFisher Scientific), transferred onto a PVDF membrane, followed by Western Blotting following standard procedure. The following primary antibodies were used for Western blotting: RACK1 (BD Biosciences, 1:1,000) and loading control α-tubulin (Protein Tech, 1:20,000). Band intensities were quantified using ImageJ. Results are shown in FIG. 30 and FIG. 31. As can be seen, a decrease in RACK1 expression is seen in ASO-treated cells compared to untreated cells, in particular in cells treated with ASO #4, #9 or #10. ASO #4 was effective in decreasing RACK1 expression at low dose (0.25 μM), thus was not investigated at higher doses (0.5 μM or 1 μM).

### In Vivo Study

ASOs #9 and #10 were selected for study. 200 uM of ASO in a 1.0 uL volume was unilaterally injected directly into the right striatum of 6 mice, 2 for each ASO, namely ASO #9 or ASO #10, or negative control ASO, with the left striatum of each mouse brain serving as an uninjected control. 7 days post-injection, the striata were micro-dissected and homogenized using a stand-up homogenizer in 200 ul of Radioimmunoprecipitation assay (RIPA) buffer (50 mM Tris pH7.5; 150 mM NaCl; 1% Triton-X-100; 1% deoxycholic acid; 0.1% SDS; 1 mM EDTA) supplemented with a protease and phosphatase inhibitor cocktail (Thermo). Samples were centrifuged at 4 C for 5 min at 14,000 rpm, and the protein concentrations of the supernatant were estimated by BCA. 25 ug of each sample was separated on 4-12% NuPage SDS-PAGE. For Western Blotting analyses, the following antibodies were used: RACK1 (BD Biosciences, 1:1,000), a-Tubulin (loading control, ProteinTech, 1:50,000). ImageJ was used to quantify band intensity.

Injection of ASO #9 or ASO #10 in the right striatum resulted in less RACK1 compared to injection of control ASO as measured by western blot and normalized to tubulin expression.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### REFERENCES

1. UniProtKB—P63244 (RACK1_HUMAN)
2. Russo A et al. (2017), “Increased cytoplasmic TDP-43 reduces global protein synthesis by interacting with RACK1 on polyribosomes”. Hum Mol Genet. 26(8):1407-1418
3. U.S. Pat. No. 8,916,530
4. Adams D R, Ron D, and Kiely PA (2011) RACK1, A multifaceted scaffolding protein: Structure and function. Cell Commun Signal. 9: 22. Review
5. Mackenzie IRA and Rademakers, R., (2008) The role of TDP-43 in amyotrophic lateral sclerosis and frontotemporal dementia Curr Opin Neurol. 21:693-700.
6. Lagier-Tourenne C., Cleveland D. W. (2009) Rethinking A LS: the FUS about TDP-43. Cell, 136, 1001-1004.
7. Zhou, Zhuan, et al. “Human rhomboid family-1 suppresses oxygen-independent degradation of hypoxia-inducible factor-1a in breast cancer.” Cancer research 74.10 (2014): 2719-2730.
8. Kraus, Sarah, et al. “Receptor for activated C kinase 1 (RACK1) and Src regulate the tyrosine phosphorylation and function of the androgen receptor.” Cancer research 66.22 (2006): 11047-11054.
9. Cao, Junxia, et al. “RACK1 Promotes Self-Renewal and Chemoresistance of Cancer Stem Cells in Human Hepatocellular Carcinoma through Stabilizing Nanog.” Theranostics 9.3 (2019): 811.
10. Culver B P, Savas J N, Park S K, Choi J H, Zheng S, Zeitlin S O, Yates J R 3rd, Tanese N. Proteomic analysis of wild-type and mutant huntingtin-associated proteins in mouse brains identifies unique interactions and involvement in protein synthesis. J Biol Chem. 2012 June 22,287(26):21599-614.
11. Mackenzie I R A et al. (2011) Distinct pathological subtypes of FTLD-FUS Acta Neurologica 121:207-218.
12. Ivone G. Bruno, Wei Jin, Gilbert J. Cote, Correction of aberrant FGFR1 alternative RNA splicing through targeting of intronic regulatory elements, Human Molecular Genetics, Volume 13, Issue 20, 15 Oct. 2004, Pages 2409-2420.
Elden A C, Kim H-J, Hart M P, Chen-Plotkin A S, Johnson B S, Fang X, Armakola M, Geser F, Greene R, Lu M M, Padmanabhan A, Clay D, McCluskey L, Elman L, Juhr D, Gruber P J, Rub U, Auburger G, Trojanowski J Q, Lee V M-Y, Van Deerlin V M, Bonini N M, Gitler A D (2010). Ataxin-2 intermediate-length polyglutamine expansions are associated with increased risk for ALS. Nature 466(7310): 1069-1075. doi:10.1038/nature09320.
Li Y, Raya P, Raoc E J, Shia C, Guoa W, Chen X, Woodruff E A III, Fushimia K, Wua J Y (2010). A Drosophila model for TDP-43 proteinopathy. PNAS 107(7): 3169-3174
Perkins, L. A., Holderbaum, L., Tao, R., Hu, Y., Sopko, R., McCall, K., Yang-Zhou, D., Flockhart, I., Binari, R., Shim, H. S., Miller, A., Housden, A., Foos, M., Randkelv, S., Kelley, C., Namgyal, P., Villalta, C., Liu, L. P., Jiang, X., Huan-Huan, Q., Wang, X., Fujiyama, A., Toyoda, A., Ayers, K., Blum, A., Czech, B., Neumuller, R., Yan, D., Cavallaro, A., Hibbard, K., Hall, D., Cooley, L., Hannon, G. J., Lehmann, R., Parks, A., Mohr, S. E., Ueda, R., Kondo, S., Ni, J. Q., Perrimon, N. (2015). The Transgenic R NAi Project at Harvard Medical School: Resources and Validation. Genetics 201(3): 843-852.
Rodriguez A del V, Didiano D, Desplan C (2012). Power tools for gene expression and clonal analysis in Drosophila. Nat Methods. 9(1):47-55. doi:10.1038/nmeth.1800.Power.
Pinarbasi, E. S., Cagatay, T., Fung, H. Y. J. et al. Active nuclear import and passive nuclear export are the primary determinants of TDP-43 localization. Sci Rep 8, 7083 (2018).

1. An oligomeric compound comprising a portion that is complementary to at least part of a nucleic acid target sequence selected from any one of SEQ ID NOs: 1-16, 49-51 and 289-499, preferably wherein the nucleic acid target sequence is selected from any one of SEQ ID NOs: 292, 297, 298, 2 and 3.

2. The oligomeric compound of claim 1, wherein the oligomeric compound is 14 to 40 nucleotides in length.

3. The oligomeric compound of claim 1 or 2, wherein the nucleic acid target sequence is selected from any one of SEQ ID NOs: 2-6, 8, 10-16, 49-51, 292-294 and 296-489.

4. The oligomeric compound of claim 1 or 2, wherein the nucleic acid target sequence is selected from any one of SEQ ID NOs: 2-6, 8, 10-16, 292-294 and 296-298.

5. The oligomeric compound of claim 1 or 2, wherein the nucleic acid target sequence selected from any one of SEQ ID NOs: 2, 3, 292, 297 and 298.

6. The oligomeric compound of claim 1 or 2, wherein the portion is complementary to the nucleic acid target sequence and the nucleic acid target sequence is or comprises a sequence selected from any one of SEQ ID NOs: 2-6, 8, 10-16, 49-51, 292-294 and 296-489.

7. The oligomeric compound of claim 1 or 2, wherein the portion is complementary to the nucleic acid target sequence and the nucleic acid target sequence is or comprises a sequence selected from any one of SEQ ID NOs: 2-6, 8, 10-16, 49-51, 292-294 and 296-298.

8. The oligomeric compound of claim 1 or 2, wherein the portion is complementary to the nucleic acid target sequence and the nucleic acid target sequence is or comprises any one of SEQ ID NOs: 2, 3, 292, 297 and 298.

9. The oligomeric compound of any one of claims 1 to 8, wherein the oligomeric compound comprises or is RNA, DNA or a mixture of DNA/RNA.

10. The oligomeric compound of any one of claims 1 to 9, comprising one or more modified nucleotide.

11. The oligomeric compound of any one of claim 9, comprising a plurality of modified nucleotides, optionally wherein all of the nucleotides of the portion are modified nucleotides.

12. The oligomeric compound of claim 10 or 11, wherein the modification is a chemical modification at a 2′ position of the ribose sugar.

13. The oligomeric compound of claim 12, wherein the chemical modification is selected from 2′O-methyl (2′-O-Me), 2′-O-methoxyethyl(2′O-MOE), 2′fluoro (2′F) and 2′-0,4′-C methylene bridge.

14. The oligomeric compound of any one of claims 1 to 3, wherein the oligomeric compound comprises at least one modified internucleoside linkage.

15. The oligomeric compound of claim 14, wherein the modified internucleoside linkage is a phosphorothioate linkage or a phosphoramidate linkage.

16. The oligomeric compound of any one of claims 10 to 15, wherein the oligomeric compound comprises a plurality of locked nucleic acid monomers (LNAM).

17. The oligomeric compound of any one of claims 1 to 16, wherein the oligomeric compound is single stranded DNA, RNA or DNA/RNA hybrid.

18. The oligomeric compound of any one of claims 1 to 16, wherein the oligomeric compound is double stranded DNA, RNA or DNA/RNA hybrid.

19. The oligomeric compound of any one of claims 1 to 18, wherein the oligomeric compound is an antisense oligonucleotide, an anti-RACK1 small interfering RNA (siRNA) or a short hairpin RNA (shRNA) construct.

20. The oligomeric compound of any one of claims 1 to 19, wherein the portion comprises a sequence of any one of SEQ ID NOs: 17-32, 52-54 and 78-288.

21. The oligomeric compound of any one of claims 1 to 19, wherein the portion comprises a sequence of any one of SEQ ID NOs: 18-22, 24, 26-32, 52-54 and 78-288.

22. The oligomeric compound of any one of claims 1 to 19, wherein the portion comprises a sequence of any one of SEQ ID NOs: 18-22, 24, 26-32, 52-54, 81-83 and 85-87.

23. The oligomeric compound of any one of claims 1 to 19, wherein the portion comprises a sequence of any one of SEQ ID NOs: 18, 19, 81, 86 and 87.

24. The oligomeric compound of any one of claims 19 to 23, wherein the oligomeric compound is an antisense oligonucleotide.

25. The oligomeric compound of claim 24, wherein the antisense oligonucleotide is a locked nucleic acid (LNA), a morpholino oligonucleotide, a gapmer or a mixmer, optionally a LNA/RNA mixmer.

26. The oligomeric compound of claim 25, wherein the portion comprises a sequence of any one of SEQ ID NOs: 78-288.

27. The oligomeric compound of claim 28, wherein the portion comprises a sequence of any one of SEQ ID NOs: 81-83 and 85-288

28. The oligomeric compound of claim 30, wherein the portion comprises a sequence of any one of SEQ ID NOs: 81, 86 and 87.

29. The oligomeric compound of claim 28, wherein the portion comprises or is SEQ ID NO: 81.

30. The oligomeric compound of claim 28, wherein the portion comprises or is SEQ ID NO: 86.

31. The oligomeric compound of claim 28, wherein the portion comprises or is SEQ ID NO: 87.

32. The oligomeric compound of any one of claims 24 to 31, wherein the antisense oligonucleotide is a gapmer comprising a plurality of DNA nucleotides flanked by a plurality of RNA nucleotides.

33. The oligomeric compound of claim 32, wherein the gapmer comprises 10 DNA nucleotides flanked by 5 RNA nucleotides on either sides.

34. The oligomeric compound of claim 32, wherein one or more of the RNA nucleotides comprises a 2′O-MOE modification, optionally wherein all of the RNA nucleotides comprise a 2′O-MOE modification.

35. The oligomeric compound of any one of claims 24 to 34, wherein the portion comprises a one or more phosphorothioate internucleoside linkages, optionally wherein all internucleoside linkages are phosphorothioate linkages.

36. The oligomeric compound of any one of claims 19 to 23, wherein the oligomeric compound is a small interfering RNA (siRNA) and the portion is a guide strand.

37. The oligomeric compound of claim 36, wherein the guide strand comprises a sequence of any one of SEQ ID NOs:17-32 and 52-54.

38. The oligomeric compound of claim 36 or 37, wherein the nucleic acid target sequence comprises 2 or more additional contiguous residues of RACK1 target sequence, optionally 19 to 30 RACK1 target sequence residues or any number in between.

39. The oligomeric compound of any one of claims 36 to 38, wherein the guide strand comprises 2 or more additional non-target residues.

40. The oligomeric compound of claim 37, wherein the guide strand comprises a sequence of SEQ ID NO: 18 with a 3′ AU overhang; SEQ ID NO: 19 with a 3′ AC overhang or SEQ ID NO: 19 with a 3′ gu overhang.

41. The oligomeric compound of claim 40, wherein the oligomeric compound is double stranded and comprises the sequences of SEQ ID NO: 40 with a 3′ au overhang and SEQ ID NO:
18 with a 3′ AU overhang.

42. The oligomeric compound of claim 40, wherein the oligomeric compound is double stranded and comprises the sequences of SEQ ID NO: 35 with a 3′ gu overhang and SEQ ID NO:
19 with a 3′ gu overhang.

43. The oligomeric compound of claim any one of claims 36 to 42, wherein the guide strand is 21-25 residues and optionally the oligomeric compound comprises a passenger strand complementary to the guide strand.

44. The oligomeric compound of any one of claims 19 to 23, wherein the oligomeric compound is shRNA.

45. The oligomeric compound of claim 44, wherein the shRNA comprises a sequence comprising 5′-3′: GAACUGAAGCAAGAAGUUAUC(SEQ ID NO: 34)(loop) GAUAACUUCUUGCUUCAGUUC(SEQ ID NO: 18) or 5′-3′: CUCUGGAUCUCGAGAUAAA (SEQ ID NO: 35)(loop) UUUAUCUCGAGAUCCAGAG (SEQ ID NO: 19).

46. The oligomeric compound of any one of claims 1 to 46, further comprising one or more cell penetrating moiety.

47. The oligomeric compound of claim 47, wherein the one or more cell penetrating moiety is a sugar, preferably N-acetylgalactosamine, a lipid, preferably cholesterol, an antibody or fragment thereof, preferably a Fab fragment, an aptamer or a peptide.

48. The oligomeric compound any one of claims 1 to 46, wherein the oligomeric compound is comprised in a vector, for example a plasmid, or viral vector such as a lentiviral vector an adenoviral vector or an adeno associated viral (AAV) vector.

49. A vector comprising the oligomeric compound of any one of claims 1 to 45.

50. The vector of claim 49, wherein the vector is selected from a plasmid and a viral vector, optionally adeno-associated virus (AAV), an adenovirus, a lentivirus, or a γ-retroviral vector.

51. A composition comprising the oligomeric compound of any one of claims 1 to 48, or the vector of claim 49 or 50, optionally comprising a diluent.

52. The composition of claim 51, comprising lipid particles such as liposomes, nanoparticles or nanosomes.

53. The composition of claim 51 or 52 comprising multiple oligomeric compounds, for example 2, 3 4 or more.

54. The composition of any one of claims 51 to 53, further comprising other antisense molecules for targeting RACK1.

55. A method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising knocking down RACK1 in neurons, astrocyte cells or microglial cells of a subject in need thereof.

56. A method of treating a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE), the method comprising administering to a subject in need thereof an effective amount of one or more antisense molecule(s) targeting RACK1.

57. The method of claim 55 or 56, wherein the one or more antisense molecule is administered by intrathecal, intracerebroventricular, intranasal, intravascular or intraparenchymal administration, preferably by intrathecal administration.

58. A method of reducing or inhibiting TDP-43 and/or FUS aggregation in a cell such as a disease cell comprising TDP-43 and/or FUS aggregation, the method comprising administering to the cell or introducing into the cell one or more antisense molecule(s) targeting RACK1 in a sufficient amount and for a sufficient time to decrease RACK1 levels in the cell.

59. The method of claim 58, wherein the amount and/or time is sufficient to reduce TDP-43 aggregation and/or restore nuclear TDP-43.

60. The method of claim 58, the amount and/or time is sufficient to reduce FUS aggregation and/or restore nuclear FUS.

61. The method of any one of claims 55 to 60, wherein the one or more antisense molecule(s) is or comprises one or more of the oligomeric compounds of any one of claims 1 to 48, optionally wherein each of the one or more are comprised in the vector of claim 49 or 50.

62. The method of any one of claims 55 to 61, wherein the one or more antisense molecules targets a nucleic acid target sequence listed in Table 1.

63. The method of any one of claims 55 to 62, the one or more antisense molecule(s) is introduced via the aforementioned composition of claims 51 to 54.

64. The method of any one of claims 55 to 63, wherein the antisense molecule and/or composition is administered or introduced into a cell naked, together with a transport reagent, or as a recombinant plasmid or viral vector that expresses the antisense molecule.

65. The method of claim 63, wherein the transport reagent comprises lipid particles such as liposomes, nanoparticles, or nanosomes.

66. The method of any one of claims 58 to 65, wherein the cell of the central nervous system, optionally a neuron, an astrocyte or a microglial cell.

67. The method of any one of claims 58 to 66, wherein the cell is in a subject, with a TDP43-opathy or a FUS-opathy neurodegenerative disease.

68. The method of claim 67, wherein the TDP43-opathy neurodegenerative disease is amyotrophic lateral sclerosis (ALS), Alzheimer's disease (AD), frontotemporal lobar dementia (FTLD), Huntington's disease (HD) or limbic-predominant age-related TDP-43 encephalopathy (LATE).

69. The method of claim 67, wherein the FUS-opathy neurodegenerative disease is neuronal intermediate filament inclusion disease (NIFID) or basophilic inclusion body disease (BIBD).

70. Use of one or more antisense molecules, optionally the oligomeric compounds of any one of claims 1 to 48, the vector of claim 49 or 50, and/or the methods of any one of claims 55 to 69, to treat amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE) in a subject in need thereof, or to reduce or inhibit TDP-43 and/or FUS aggregation in a cell such as a neuron, an astrocyte or a microglial cell.

71. An antisense molecule, an oligomeric compound of any one of claims 1 to 48, a vector of claim 49 to 50 or a composition of any one of claims 51 to 54 for use in the treatment of a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE).

72. Use of an antisense molecule, an oligomeric compound of any one of claims 1 to 48, a vector of claim 49 to 50 or a composition of any one of claims 51 to 54 for the manufacture of a medicament for the treatment of a TDP43-opathy or a FUS-opathy neurodegenerative disease optionally selected from amyotrophic lateral sclerosis (ALS), Alzheimer's disease (AD), frontotemporal lobar dementia (FTLD), Huntington's disease (HD), neuronal intermediate filament inclusion disease (NIFID), basophilic inclusion body disease (BIBD) or limbic-predominant age-related TDP-43 encephalopathy (LATE).


SEQUENCE LISTING (502 sequences)

SEQ ID NO: 1 - DNA, 19 bp, Homo Sapiens
tttagagggaaagatcatt

SEQ ID NO: 2 - DNA, 21 bp, Homo Sapiens
gaactgaagcaagaagttatc

SEQ ID NO: 3 - DNA, 19 bp, Homo Sapiens
ctctggatctcgagataaa

SEQ ID NO: 4 - DNA, 19 bp, Homo Sapiens
gctaactgcaagctgaaga

SEQ ID NO: 5 - DNA, 19 bp, Homo Sapiens
gacaagctggtcaaggtat

SEQ ID NO: 6 - DNA, 19 bp, Homo Sapiens
ggatggccaggccatgtta

SEQ ID NO: 7 - DNA, 19 bp, Homo Sapiens
acacctttacacgctagat

SEQ ID NO: 8 - DNA, 19 bp, Homo Sapiens
ctatctgaacacggtgact

SEQ ID NO: 9 - DNA, 19 bp, Homo Sapiens
cagggatgagaccaactat

SEQ ID NO: 10 - DNA, 19 bp, Homo Sapiens
ccaacagcagcaaccctat

SEQ ID NO: 11 - DNA, 19 bp, Homo Sapiens
ctttgttagtgatgtggtt

SEQ ID NO: 12 - DNA, 19 bp, Homo Sapiens
ccctgggtgtgtgcaaata

SEQ ID NO: 13 - DNA, 19 bp, Homo Sapiens
gctgatggccagactctgt

SEQ ID NO: 14 - DNA, 19 bp, Homo Sapiens
gatttgtgggccataccaa

SEQ ID NO: 15 - DNA, 19 bp, Homo Sapiens
gtaacccagatcgctacta

SEQ ID NO: 16 - DNA, 19 bp, Homo Sapiens
cgcagttcccggacatgat

SEQ ID NO: 17 - RNA, 19 nt, Artificial Sequence
  Synthetic Construct
aaugaucuuucccucuaaa

SEQ ID NO: 18 - RNA, 21 nt, Artificial Sequence
  Synthetic Construct
gauaacuucuugcuucaguuc

SEQ ID NO: 19 - RNA, 19 nt, Artificial Sequence
  Synthetic Construct
uuuaucucgagauccagag

SEQ ID NO: 20 - RNA, 19 nt, Artificial Sequence
  Synthetic Construct
ucuucagcuugcaguuagc

SEQ ID NO: 21 - RNA, 19 nt, Artificial Sequence
  Synthetic Construct
auaccuugaccagcuuguc

SEQ ID NO: 22 - RNA, 19 nt, Artificial Sequence
  Synthetic Construct
uaacauggccuggccaucc

SEQ ID NO: 23 - RNA, 19 nt, Artificial Sequence
  Synthetic Construct
aucuagcguguaaaggugu

SEQ ID NO: 24 - RNA, 19 nt, Artificial Sequence
  Synthetic Construct
agucaccguguucagauag

SEQ ID NO: 25 - RNA, 19 nt, Artificial Sequence
  Synthetic Construct
auaguuggucucaucccug

SEQ ID NO: 26 - RNA, 19 nt, Artificial Sequence
  Synthetic Construct
auaggguugcugcuguugg

SEQ ID NO: 27 - RNA, 19 nt, Artificial Sequence
  Synthetic Construct
aaccacaucacuaacaaag

SEQ ID NO: 28 - RNA, 19 nt, Artificial Sequence
  Synthetic Construct
uauuugcacacacccaggg

SEQ ID NO: 29 - RNA, 19 nt, Artificial Sequence
  Synthetic Construct
acagagucuggccaucagc

SEQ ID NO: 30 - RNA, 19 nt, Artificial Sequence
  Synthetic Construct
uugguauggcccacaaauc

SEQ ID NO: 31 - RNA, 19 nt, Artificial Sequence
  Synthetic Construct
uaguagcgaucuggguuac

SEQ ID NO: 32 - RNA, 19 nt, Artificial Sequence
  Synthetic Construct
aucauguccgggaacugcg

SEQ ID NO: 33 - RNA, 19 nt, Artificial Sequence
  Synthetic Construct
uuuagagggaaagaucauu

SEQ ID NO: 34 - RNA, 21 nt, Artificial Sequence
  Synthetic Construct
gaacugaagcaagaaguuauc

SEQ ID NO: 35 - RNA, 19 nt, Artificial Sequence
  Synthetic Construct
cucuggaucucgagauaaa

SEQ ID NO: 36 - RNA, 19 nt, Artificial Sequence
  Synthetic Construct
gcuaacugcaagcugaaga

SEQ ID NO: 37 - RNA, 19 nt, Artificial Sequence
  Synthetic Construct
gacaagcuggucaagguau

SEQ ID NO: 38 - RNA, 19 nt, Artificial Sequence
  Synthetic Construct
ggauggccaggccauguua

SEQ ID NO: 39 - RNA, 19 nt, Artificial Sequence
  Synthetic Construct
acaccuuuacacgcuagau

SEQ ID NO: 40 - RNA, 19 nt, Artificial Sequence
  Synthetic Construct
cuaucugaacacggugacu

SEQ ID NO: 41 - RNA, 19 nt, Artificial Sequence
  Synthetic Construct
cagggaugagaccaacuau

SEQ ID NO: 42 - RNA, 19 nt, Artificial Sequence
  Synthetic Construct
ccaacagcagcaacccuau

SEQ ID NO: 43 - RNA, 19 nt, Artificial Sequence
  Synthetic Construct
cuuuguuagugaugugguu

SEQ ID NO: 44 - RNA, 19 nt, Artificial Sequence
  Synthetic Construct
cccugggugugugcaaaua

SEQ ID NO: 45 - RNA, 19 nt, Artificial Sequence
  Synthetic Construct
gcugauggccagacucugu

SEQ ID NO: 46 - RNA, 19 nt, Artificial Sequence
  Synthetic Construct
gauuugugggccauaccaa

SEQ ID NO: 47 - RNA, 19 nt, Artificial Sequence
  Synthetic Construct
guaacccagaucgcuacua

SEQ ID NO: 48 - RNA, 19 nt, Artificial Sequence
  Synthetic Construct
cgcaguucccggacaugau

SEQ ID NO: 49 - DNA, 19 bp, Homo Sapiens
gtacggactaaggtagatt

SEQ ID NO: 50 - DNA, 19 bp, Homo Sapiens
ttttacctcctttagataa

SEQ ID NO: 51 - DNA, 19 bp, Homo Sapiens
tgttccccaggatttagag

SEQ ID NO: 52 - RNA, 19 nt, Artificial Sequence
  Synthetic Construct
aaucuaccuuaguccguac

SEQ ID NO: 53 - RNA, 19 nt, Artificial Sequence
  Synthetic Construct
uuaucuaaaggagguaaaa

SEQ ID NO: 54 - RNA, 19 nt, Artificial Sequence
  Synthetic Construct
cucuaaauccuggggaaca

SEQ ID NO: 55 - RNA, 19 nt, Artificial Sequence
  Synthetic Construct
guacggacuaagguagauu

SEQ ID NO: 56 - RNA, 19 nt, Artificial Sequence
  Synthetic Construct
uuuuaccuccuuuagauaa

SEQ ID NO: 57 - RNA, 19 nt, Artificial Sequence
  Synthetic Construct
uguuccccaggauuuagag

SEQ ID NO: 58 - DNA, 19 bp, Homo Sapiens
ccgccatgactgagcagat

SEQ ID NO: 59 - DNA, 19 bp, Homo Sapiens
cctgcgcctctgggatctc

SEQ ID NO: 60 - DNA, 19 bp, Homo Sapiens
tcagagtgggtgtcttgtg

SEQ ID NO: 61 - DNA, 19 bp, Homo Sapiens
ccctaaccgctactggctg

SEQ ID NO: 62 - RNA, 19 nt, Artificial Sequence
  Synthetic Construct
aucugcucagucauggcgg

SEQ ID NO: 63 - RNA, 19 nt, Artificial Sequence
  Synthetic Construct
gagaucccagaggcgcagg

SEQ ID NO: 64 - RNA, 19 nt, Artificial Sequence
  Synthetic Construct
cacaagacacccacucuga

SEQ ID NO: 65 - RNA, 19 nt, Artificial Sequence
  Synthetic Construct
cagccaguagcgguuaggg

SEQ ID NO: 66 - RNA, 19 nt, Artificial Sequence
  Synthetic Construct
ccgccaugacugagcagau

SEQ ID NO: 67 - RNA, 19 nt, Artificial Sequence
  Synthetic Construct
ccugcgccucugggaucuc

SEQ ID NO: 68 - RNA, 19 nt, Artificial Sequence
  Synthetic Construct
ucagagugggugucuugug

SEQ ID NO: 69 - RNA, 19 nt, Artificial Sequence
  Synthetic Construct
cccuaaccgcuacuggcug

SEQ ID NO: 70 - DNA, 19 bp, Homo Sapiens
accagggatgagaccaact

SEQ ID NO: 71 - DNA, 19 bp, Homo Sapiens
gggatgagaccaactatgg

SEQ ID NO: 72 - DNA, 21 bp, Homo Sapiens
ggatgagaccaactatggaat

SEQ ID NO: 73 - DNA, 19 bp, Homo Sapiens
ggtatggaacctggctaac

SEQ ID NO: 74 - DNA, 19 bp, Homo Sapiens
gggaaagatcattgtagat

SEQ ID NO: 75 - DNA, 19 bp, Homo Sapiens
cctttacacgctagatggt

SEQ ID NO: 76 - DNA, 21 bp, Artificial Sequence
  Synthetic Construct
caagaccatcaagctgtggaa

SEQ ID NO: 77 - DNA, 21 bp, Artificial Sequence
  Synthetic Construct
ttccacagcttgatggtcttg

SEQ ID NO: 78 - DNA, 20 bp, Artificial Sequence
  Synthetic Oligonucleotide
tggtctcatccctggtcagt

SEQ ID NO: 79 - DNA, 20 bp, Artificial Sequence
  Synthetic Oligonucleotide
cacgaagggtcatctgctca

SEQ ID NO: 80 - DNA, 20 bp, Artificial Sequence
  Synthetic Oligonucleotide
caggttccataccttgacca

SEQ ID NO: 81 - DNA, 20 bp, Artificial Sequence
  Synthetic Oligonucleotide
tccagagacaatctgccggt

SEQ ID NO: 82 - DNA, 20 bp, Artificial Sequence
  Synthetic Oligonucleotide
accgtgttcagatagcctgt

SEQ ID NO: 83 - DNA, 20 bp, Artificial Sequence
  Synthetic Oligonucleotide
atcatgtccgggaactgcgg

SEQ ID NO: 84 - DNA, 20 bp, Artificial Sequence
  Synthetic Oligonucleotide
cgttgtgagatcccagaggc

SEQ ID NO: 85 - DNA, 20 bp, Artificial Sequence
  Synthetic Oligonucleotide
gccggttgtcagaggagaag

SEQ ID NO: 86 - DNA, 20 bp, Artificial Sequence
  Synthetic Oligonucleotide
ccagagacaatctgccggtt

SEQ ID NO: 87 - DNA, 20 bp, Artificial Sequence
  Synthetic Oligonucleotide
acgatgatagggttgctgct

SEQ ID NO: 88 - DNA, 20 bp, Artificial Sequence
  Synthetic Oligonucleotide
aagggtcatctgctcagtca

SEQ ID NO: 89 - DNA, 20 bp, Artificial Sequence
  Synthetic Oligonucleotide
gaagggtcatctgctcagtc

SEQ ID NO: 90 - DNA, 20 bp, Artificial Sequence
  Synthetic Oligonucleotide
cgaagggtcatctgctcagt

SEQ ID NO: 91 - DNA, 20 bp, Artificial Sequence
  Synthetic Oligonucleotide
acgaagggtcatctgctcag

SEQ ID NO: 92 - DNA, 20 bp, Artificial Sequence
  Synthetic Oligonucleotide
ccacgaagggtcatctgctc

SEQ ID NO: 93 - DNA, 20 bp, Artificial Sequence
  Synthetic Oligonucleotide
gccacgaagggtcatctgct

SEQ ID NO: 94 - DNA, 20 bp, Artificial Sequence
  Synthetic Oligonucleotide
tgccacgaagggtcatctgc

SEQ ID NO: 95 - DNA, 20 bp, Artificial Sequence
  Synthetic Oligonucleotide
gtgccacgaagggtcatctg

SEQ ID NO: 96 - DNA, 20 bp, Artificial Sequence
  Synthetic Oligonucleotide
ggtgccacgaagggtcatct

SEQ ID NO: 97 - DNA, 20 bp, Artificial Sequence
  Synthetic Oligonucleotide
gggtgccacgaagggtcatc

SEQ ID NO: 98 - DNA, 20 bp, Artificial Sequence
  Synthetic Oligonucleotide
agggtgccacgaagggtcat

SEQ ID NO: 99 - DNA, 20 bp, Artificial Sequence
  Synthetic Oligonucleotide
gagggtgccacgaagggtca

SEQ ID NO: 100 - DNA, 20 bp, Artificial Sequence
  Synthetic Oligonucleotide
tgagggtgccacgaagggtc

SEQ ID NO: 101 - DNA, 20 bp, Artificial Sequence
  Synthetic Oligonucleotide
ttgagggtgccacgaagggt

SEQ ID NO: 102 - DNA, 20 bp, Artificial Sequence
  Synthetic Oligonucleotide
cttgagggtgccacgaaggg

SEQ ID NO: 103 - DNA, 20 bp, Artificial Sequence
  Synthetic Oligonucleotide
ccttgagggtgccacgaagg

SEQ ID NO: 104 - DNA, 20 bp, Artificial Sequence
  Synthetic Oligonucleotide
cccttgagggtgccacgaag

SEQ ID NO: 105 - DNA, 20 bp, Artificial Sequence
  Synthetic Oligonucleotide
gcccttgagggtgccacgaa

SEQ ID NO: 106 - DNA, 20 bp, Artificial Sequence
  Synthetic Oligonucleotide
ggcccttgagggtgccacga

SEQ ID NO: 107 - DNA, 20 bp, Artificial Sequence
  Synthetic Oligonucleotide
tgcggggtagtagcgatctg

SEQ ID NO: 108 - DNA, 20 bp, Artificial Sequence
  Synthetic Oligonucleotide
ctgcggggtagtagcgatct

SEQ ID NO: 109 - DNA, 20 bp, Artificial Sequence
  Synthetic Oligonucleotide
actgcggggtagtagcgatc

SEQ ID NO: 110 - DNA, 20 bp, Artificial Sequence
  Synthetic Oligonucleotide
aactgcggggtagtagcgat

SEQ ID NO: 111 - DNA, 20 bp, Artificial Sequence
  Synthetic Oligonucleotide
gaactgcggggtagtagcga

SEQ ID NO: 112 - DNA, 20 bp, Artificial Sequence
  Synthetic Oligonucleotide
ggaactgcggggtagtagcg

SEQ ID NO: 113 - DNA, 20 bp, Artificial Sequence
  Synthetic Oligonucleotide
gggaactgcggggtagtagc

SEQ ID NO: 114 - DNA, 20 bp, Artificial Sequence
  Synthetic Oligonucleotide
cgggaactgcggggtagtag

SEQ ID NO: 115 - DNA, 20 bp, Artificial Sequence
  Synthetic Oligonucleotide
ccgggaactgcggggtagta

SEQ ID NO: 116 - DNA, 20 bp, Artificial Sequence
  Synthetic Oligonucleotide
tccgggaactgcggggtagt

SEQ ID NO: 117 - DNA, 20 bp, Artificial Sequence
  Synthetic Oligonucleotide
gtccgggaactgcggggtag

SEQ ID NO: 118 - DNA, 20 bp, Artificial Sequence
  Synthetic Oligonucleotide
tgtccgggaactgcggggta

SEQ ID NO: 119 - DNA, 20 bp, Artificial Sequence
  Synthetic Oligonucleotide
atgtccgggaactgcggggt

SEQ ID NO: 120 - DNA, 20 bp, Artificial Sequence
  Synthetic Oligonucleotide
catgtccgggaactgcgggg

SEQ ID NO: 121 - DNA, 20 bp, Artificial Sequence
  Synthetic Oligonucleotide
tcatgtccgggaactgcggg

SEQ ID NO: 122 - DNA, 20 bp, Artificial Sequence
  Synthetic Oligonucleotide
gatcatgtccgggaactgcg

SEQ ID NO: 123 - DNA, 20 bp, Artificial Sequence
  Synthetic Oligonucleotide
ggatcatgtccgggaactgc

SEQ ID NO: 124 - DNA, 20 bp, Artificial Sequence
  Synthetic Oligonucleotide
aggatcatgtccgggaactg

SEQ ID NO: 125 - DNA, 20 bp, Artificial Sequence
  Synthetic Oligonucleotide
gaggatcatgtccgggaact

SEQ ID NO: 126 - DNA, 20 bp, Artificial Sequence
  Synthetic Oligonucleotide
agaggatcatgtccgggaac

SEQ ID NO: 127 - DNA, 20 bp, Artificial Sequence
  Synthetic Oligonucleotide
gagaggatcatgtccgggaa

SEQ ID NO: 128 - DNA, 20 bp, Artificial Sequence
  Synthetic Oligonucleotide
ggagaggatcatgtccggga

SEQ ID NO: 129 - DNA, 20 bp, Artificial Sequence
  Synthetic Oligonucleotide
cggagaggatcatgtccggg

SEQ ID NO: 130 - DNA, 20 bp, Artificial Sequence
  Synthetic Oligonucleotide
gcggagaggatcatgtccgg

SEQ ID NO: 131 - DNA, 20 bp, Artificial Sequence
  Synthetic Oligonucleotide
ggcggagaggatcatgtccg

SEQ ID NO: 132 - DNA, 20 bp, Artificial Sequence
  Synthetic Oligonucleotide
aggcggagaggatcatgtcc

SEQ ID NO: 133 - DNA, 20 bp, Artificial Sequence
  Synthetic Oligonucleotide
gaggcggagaggatcatgtc

SEQ ID NO: 134 - DNA, 20 bp, Artificial Sequence
  Synthetic Oligonucleotide
agaggcggagaggatcatgt

SEQ ID NO: 135 - DNA, 20 bp, Artificial Sequence
  Synthetic Oligonucleotide
gagaggcggagaggatcatg

SEQ ID NO: 136 - DNA, 20 bp, Artificial Sequence
  Synthetic Oligonucleotide
cgagaggcggagaggatcat

SEQ ID NO: 137 - DNA, 20 bp, Artificial Sequence
  Synthetic Oligonucleotide
tcagtttccacatgatgatg

SEQ ID NO: 138 - DNA, 20 bp, Artificial Sequence
  Synthetic Oligonucleotide
gtcagtttccacatgatgat

SEQ ID NO: 139 - DNA, 20 bp, Artificial Sequence
  Synthetic Oligonucleotide
ggtcagtttccacatgatga

SEQ ID NO: 140 - DNA, 20 bp, Artificial Sequence
  Synthetic Oligonucleotide
tggtcagtttccacatgatg

SEQ ID NO: 141 - DNA, 20 bp, Artificial Sequence
  Synthetic Oligonucleotide
ctggtcagtttccacatgat

SEQ ID NO: 142 - DNA, 20 bp, Artificial Sequence
  Synthetic Oligonucleotide
cctggtcagtttccacatga

SEQ ID NO: 143 - DNA, 20 bp, Artificial Sequence
  Synthetic Oligonucleotide
ccctggtcagtttccacatg

SEQ ID NO: 144 - DNA, 20 bp, Artificial Sequence
  Synthetic Oligonucleotide
tccctggtcagtttccacat

SEQ ID NO: 145 - DNA, 20 bp, Artificial Sequence
  Synthetic Oligonucleotide
atccctggtcagtttccaca

SEQ ID NO: 146 - DNA, 20 bp, Artificial Sequence
  Synthetic Oligonucleotide
catccctggtcagtttccac

SEQ ID NO: 147 - DNA, 20 bp, Artificial Sequence
  Synthetic Oligonucleotide
tcatccctggtcagtttcca

SEQ ID NO: 148 - DNA, 20 bp, Artificial Sequence
  Synthetic Oligonucleotide
ctcatccctggtcagtttcc

SEQ ID NO: 149 - DNA, 20 bp, Artificial Sequence
  Synthetic Oligonucleotide
tctcatccctggtcagtttc

SEQ ID NO: 150 - DNA, 20 bp, Artificial Sequence
  Synthetic Oligonucleotide
gaggcgcagggttccatccc

SEQ ID NO: 151 - DNA, 20 bp, Artificial Sequence
  Synthetic Oligonucleotide
agaggcgcagggttccatcc

SEQ ID NO: 152 - DNA, 20 bp, Artificial Sequence
  Synthetic Oligonucleotide
cagaggcgcagggttccatc

SEQ ID NO: 153 - DNA, 20 bp, Artificial Sequence
  Synthetic Oligonucleotide
ccagaggcgcagggttccat

SEQ ID NO: 154 - DNA, 20 bp, Artificial Sequence
  Synthetic Oligonucleotide
ccgttgtgagatcccagagg

SEQ ID NO: 155 - DNA, 20 bp, Artificial Sequence
  Synthetic Oligonucleotide
cccgttgtgagatcccagag

SEQ ID NO: 156 - DNA, 20 bp, Artificial Sequence
  Synthetic Oligonucleotide
gcccgttgtgagatcccaga

SEQ ID NO: 157 - DNA, 20 bp, Artificial Sequence
  Synthetic Oligonucleotide
tgcccgttgtgagatcccag

SEQ ID NO: 158 - DNA, 20 bp, Artificial Sequence
  Synthetic Oligonucleotide
gtgcccgttgtgagatccca

SEQ ID NO: 159 - DNA, 20 bp, Artificial Sequence
  Synthetic Oligonucleotide
ggtgcccgttgtgagatccc

SEQ ID NO: 160 - DNA, 20 bp, Artificial Sequence
  Synthetic Oligonucleotide
tggtgcccgttgtgagatcc

SEQ ID NO: 161 - DNA, 20 bp, Artificial Sequence
  Synthetic Oligonucleotide
gtggtgcccgttgtgagatc

SEQ ID NO: 162 - DNA, 20 bp, Artificial Sequence
  Synthetic Oligonucleotide
ggtggtgcccgttgtgagat

SEQ ID NO: 163 - DNA, 20 bp, Artificial Sequence
  Synthetic Oligonucleotide
tggtggtgcccgttgtgaga

SEQ ID NO: 164 - DNA, 20 bp, Artificial Sequence
  Synthetic Oligonucleotide
gtggtggtgcccgttgtgag

SEQ ID NO: 165 - DNA, 20 bp, Artificial Sequence
  Synthetic Oligonucleotide
cgtggtggtgcccgttgtga

SEQ ID NO: 166 - DNA, 20 bp, Artificial Sequence
  Synthetic Oligonucleotide
tcgtggtggtgcccgttgtg

SEQ ID NO: 167 - DNA, 20 bp, Artificial Sequence
  Synthetic Oligonucleotide
ctcgtggtggtgcccgttgt

SEQ ID NO: 168 - DNA, 20 bp, Artificial Sequence
  Synthetic Oligonucleotide
cctcgtggtggtgcccgttg

SEQ ID NO: 169 - DNA, 20 bp, Artificial Sequence
  Synthetic Oligonucleotide
agaaggccacactcagcaca

SEQ ID NO: 170 - DNA, 20 bp, Artificial Sequence
  Synthetic Oligonucleotide
gagaaggccacactcagcac

SEQ ID NO: 171 - DNA, 20 bp, Artificial Sequence
  Synthetic Oligonucleotide
ggagaaggccacactcagca

SEQ ID NO: 172 - DNA, 20 bp, Artificial Sequence
  Synthetic Oligonucleotide
aggagaaggccacactcagc

SEQ ID NO: 173 - DNA, 20 bp, Artificial Sequence
  Synthetic Oligonucleotide
gaggagaaggccacactcag

SEQ ID NO: 174 - DNA, 20 bp, Artificial Sequence
  Synthetic Oligonucleotide
agaggagaaggccacactca

SEQ ID NO: 175 - DNA, 20 bp, Artificial Sequence
  Synthetic Oligonucleotide
cagaggagaaggccacactc

SEQ ID NO: 176 - DNA, 20 bp, Artificial Sequence
  Synthetic Oligonucleotide
tcagaggagaaggccacact

SEQ ID NO: 177 - DNA, 20 bp, Artificial Sequence
  Synthetic Oligonucleotide
gtcagaggagaaggccacac

SEQ ID NO: 178 - DNA, 20 bp, Artificial Sequence
  Synthetic Oligonucleotide
tgtcagaggagaaggccaca

SEQ ID NO: 179 - DNA, 20 bp, Artificial Sequence
  Synthetic Oligonucleotide
ttgtcagaggagaaggccac

SEQ ID NO: 180 - DNA, 20 bp, Artificial Sequence
  Synthetic Oligonucleotide
gttgtcagaggagaaggcca

SEQ ID NO: 181 - DNA, 20 bp, Artificial Sequence
  Synthetic Oligonucleotide
ggttgtcagaggagaaggcc

SEQ ID NO: 182 - DNA, 20 bp, Artificial Sequence
  Synthetic Oligonucleotide
cggttgtcagaggagaaggc

SEQ ID NO: 183 - DNA, 20 bp, Artificial Sequence
  Synthetic Oligonucleotide
ccggttgtcagaggagaagg

SEQ ID NO: 184 - DNA, 20 bp, Artificial Sequence
  Synthetic Oligonucleotide
tgccggttgtcagaggagaa

SEQ ID NO: 185 - DNA, 20 bp, Artificial Sequence
  Synthetic Oligonucleotide
ctgccggttgtcagaggaga

SEQ ID NO: 186 - DNA, 20 bp, Artificial Sequence
  Synthetic Oligonucleotide
tctgccggttgtcagaggag

SEQ ID NO: 187 - DNA, 20 bp, Artificial Sequence
  Synthetic Oligonucleotide
atctgccggttgtcagagga

SEQ ID NO: 188 - DNA, 20 bp, Artificial Sequence
  Synthetic Oligonucleotide
aatctgccggttgtcagagg

SEQ ID NO: 189 - DNA, 20 bp, Artificial Sequence
  Synthetic Oligonucleotide
caatctgccggttgtcagag

SEQ ID NO: 190 - DNA, 20 bp, Artificial Sequence
  Synthetic Oligonucleotide
acaatctgccggttgtcaga

SEQ ID NO: 191 - DNA, 20 bp, Artificial Sequence
  Synthetic Oligonucleotide
gacaatctgccggttgtcag

SEQ ID NO: 192 - DNA, 20 bp, Artificial Sequence
  Synthetic Oligonucleotide
agacaatctgccggttgtca

SEQ ID NO: 193 - DNA, 20 bp, Artificial Sequence
  Synthetic Oligonucleotide
gagacaatctgccggttgtc

SEQ ID NO: 194 - DNA, 20 bp, Artificial Sequence
  Synthetic Oligonucleotide
agagacaatctgccggttgt

SEQ ID NO: 195 - DNA, 20 bp, Artificial Sequence
  Synthetic Oligonucleotide
cagagacaatctgccggttg

SEQ ID NO: 196 - DNA, 20 bp, Artificial Sequence
  Synthetic Oligonucleotide
atccagagacaatctgccgg

SEQ ID NO: 197 - DNA, 20 bp, Artificial Sequence
  Synthetic Oligonucleotide
gatccagagacaatctgccg

SEQ ID NO: 198 - DNA, 20 bp, Artificial Sequence
  Synthetic Oligonucleotide
agatccagagacaatctgcc

SEQ ID NO: 199 - DNA, 20 bp, Artificial Sequence
  Synthetic Oligonucleotide
gagatccagagacaatctgc

SEQ ID NO: 200 - DNA, 20 bp, Artificial Sequence
  Synthetic Oligonucleotide
cgagatccagagacaatctg

SEQ ID NO: 201 - DNA, 20 bp, Artificial Sequence
  Synthetic Oligonucleotide
tcgagatccagagacaatct

SEQ ID NO: 202 - DNA, 20 bp, Artificial Sequence
  Synthetic Oligonucleotide
ctcgagatccagagacaatc

SEQ ID NO: 203 - DNA, 20 bp, Artificial Sequence
  Synthetic Oligonucleotide
tctcgagatccagagacaat

SEQ ID NO: 204 - DNA, 20 bp, Artificial Sequence
  Synthetic Oligonucleotide
atctcgagatccagagacaa

SEQ ID NO: 205 - DNA, 20 bp, Artificial Sequence
  Synthetic Oligonucleotide
tatctcgagatccagagaca

SEQ ID NO: 206 - DNA, 20 bp, Artificial Sequence
  Synthetic Oligonucleotide
ttatctcgagatccagagac

SEQ ID NO: 207 - DNA, 20 bp, Artificial Sequence
  Synthetic Oligonucleotide
tttatctcgagatccagaga

SEQ ID NO: 208 - DNA, 20 bp, Artificial Sequence
  Synthetic Oligonucleotide
ttttatctcgagatccagag

SEQ ID NO: 209 - DNA, 20 bp, Artificial Sequence
  Synthetic Oligonucleotide
gttttatctcgagatccaga

SEQ ID NO: 210 - DNA, 20 bp, Artificial Sequence
  Synthetic Oligonucleotide
ggttttatctcgagatccag

SEQ ID NO: 211 - DNA, 20 bp, Artificial Sequence
  Synthetic Oligonucleotide
ctgctgttgggcgagaagcg

SEQ ID NO: 212 - DNA, 20 bp, Artificial Sequence
  Synthetic Oligonucleotide
gctgctgttgggcgagaagc

SEQ ID NO: 213 - DNA, 20 bp, Artificial Sequence
  Synthetic Oligonucleotide
tgctgctgttgggcgagaag

SEQ ID NO: 214 - DNA, 20 bp, Artificial Sequence
  Synthetic Oligonucleotide
ttgctgctgttgggcgagaa

SEQ ID NO: 215 - DNA, 20 bp, Artificial Sequence
  Synthetic Oligonucleotide
gttgctgctgttgggcgaga

SEQ ID NO: 216 - DNA, 20 bp, Artificial Sequence
  Synthetic Oligonucleotide
ggttgctgctgttgggcgag

SEQ ID NO: 217 - DNA, 20 bp, Artificial Sequence
  Synthetic Oligonucleotide
gggttgctgctgttgggcga

SEQ ID NO: 218 - DNA, 20 bp, Artificial Sequence
  Synthetic Oligonucleotide
agggttgctgctgttgggcg

SEQ ID NO: 219 - DNA, 20 bp, Artificial Sequence
  Synthetic Oligonucleotide
tagggttgctgctgttgggc

SEQ ID NO: 220 - DNA, 20 bp, Artificial Sequence
  Synthetic Oligonucleotide
atagggttgctgctgttggg

SEQ ID NO: 221 - DNA, 20 bp, Artificial Sequence
  Synthetic Oligonucleotide
gatagggttgctgctgttgg

SEQ ID NO: 222 - DNA, 20 bp, Artificial Sequence
  Synthetic Oligonucleotide
tgatagggttgctgctgttg

SEQ ID NO: 223 - DNA, 20 bp, Artificial Sequence
  Synthetic Oligonucleotide
atgatagggttgctgctgtt

SEQ ID NO: 224 - DNA, 20 bp, Artificial Sequence
  Synthetic Oligonucleotide
gatgatagggttgctgctgt

SEQ ID NO: 225 - DNA, 20 bp, Artificial Sequence
  Synthetic Oligonucleotide
cgatgatagggttgctgctg

SEQ ID NO: 226 - DNA, 20 bp, Artificial Sequence
  Synthetic Oligonucleotide
gacgatgatagggttgctgc

SEQ ID NO: 227 - DNA, 20 bp, Artificial Sequence
  Synthetic Oligonucleotide
agacgatgatagggttgctg

SEQ ID NO: 228 - DNA, 20 bp, Artificial Sequence
  Synthetic Oligonucleotide
gagacgatgatagggttgct

SEQ ID NO: 229 - DNA, 20 bp, Artificial Sequence
  Synthetic Oligonucleotide
ggagacgatgatagggttgc

SEQ ID NO: 230 - DNA, 20 bp, Artificial Sequence
  Synthetic Oligonucleotide
aggagacgatgatagggttg

SEQ ID NO: 231 - DNA, 20 bp, Artificial Sequence
  Synthetic Oligonucleotide
caggagacgatgatagggtt

SEQ ID NO: 232 - DNA, 20 bp, Artificial Sequence
  Synthetic Oligonucleotide
acaggagacgatgatagggt

SEQ ID NO: 233 - DNA, 20 bp, Artificial Sequence
  Synthetic Oligonucleotide
cacaggagacgatgataggg

SEQ ID NO: 234 - DNA, 20 bp, Artificial Sequence
  Synthetic Oligonucleotide
ccacaggagacgatgatagg

SEQ ID NO: 235 - DNA, 20 bp, Artificial Sequence
  Synthetic Oligonucleotide
gccacaggagacgatgatag

SEQ ID NO: 236 - DNA, 20 bp, Artificial Sequence
  Synthetic Oligonucleotide
agccacaggagacgatgata

SEQ ID NO: 237 - DNA, 20 bp, Artificial Sequence
  Synthetic Oligonucleotide
cagccacaggagacgatgat

SEQ ID NO: 238 - DNA, 20 bp, Artificial Sequence
  Synthetic Oligonucleotide
ccagccacaggagacgatga

SEQ ID NO: 239 - DNA, 20 bp, Artificial Sequence
  Synthetic Oligonucleotide
cccagccacaggagacgatg

SEQ ID NO: 240 - DNA, 20 bp, Artificial Sequence
  Synthetic Oligonucleotide
tcccagccacaggagacgat

SEQ ID NO: 241 - DNA, 20 bp, Artificial Sequence
  Synthetic Oligonucleotide
gaccagcttgtcccagccac

SEQ ID NO: 242 - DNA, 20 bp, Artificial Sequence
  Synthetic Oligonucleotide
tgaccagcttgtcccagcca

SEQ ID NO: 243 - DNA, 20 bp, Artificial Sequence
  Synthetic Oligonucleotide
ttgaccagcttgtcccagcc

SEQ ID NO: 244 - DNA, 20 bp, Artificial Sequence
  Synthetic Oligonucleotide
cttgaccagcttgtcccagc

SEQ ID NO: 245 - DNA, 20 bp, Artificial Sequence
  Synthetic Oligonucleotide
ccttgaccagcttgtcccag

SEQ ID NO: 246 - DNA, 20 bp, Artificial Sequence
  Synthetic Oligonucleotide
accttgaccagcttgtccca

SEQ ID NO: 247 - DNA, 20 bp, Artificial Sequence
  Synthetic Oligonucleotide
taccttgaccagcttgtccc

SEQ ID NO: 248 - DNA, 20 bp, Artificial Sequence
  Synthetic Oligonucleotide
ataccttgaccagcttgtcc

SEQ ID NO: 249 - DNA, 20 bp, Artificial Sequence
  Synthetic Oligonucleotide
cataccttgaccagcttgtc

SEQ ID NO: 250 - DNA, 20 bp, Artificial Sequence
  Synthetic Oligonucleotide
ccataccttgaccagcttgt

SEQ ID NO: 251 - DNA, 20 bp, Artificial Sequence
  Synthetic Oligonucleotide
tccataccttgaccagcttg

SEQ ID NO: 252 - DNA, 20 bp, Artificial Sequence
  Synthetic Oligonucleotide
ttccataccttgaccagctt

SEQ ID NO: 253 - DNA, 20 bp, Artificial Sequence
  Synthetic Oligonucleotide
gttccataccttgaccagct

SEQ ID NO: 254 - DNA, 20 bp, Artificial Sequence
  Synthetic Oligonucleotide
ggttccataccttgaccagc

SEQ ID NO: 255 - DNA, 20 bp, Artificial Sequence
  Synthetic Oligonucleotide
aggttccataccttgaccag

SEQ ID NO: 256 - DNA, 20 bp, Artificial Sequence
  Synthetic Oligonucleotide
gcttgcagttagccaggttc

SEQ ID NO: 257 - DNA, 20 bp, Artificial Sequence
  Synthetic Oligonucleotide
agcttgcagttagccaggtt

SEQ ID NO: 258 - DNA, 20 bp, Artificial Sequence
  Synthetic Oligonucleotide
cagcttgcagttagccaggt

SEQ ID NO: 259 - DNA, 20 bp, Artificial Sequence
  Synthetic Oligonucleotide
cctgtgtggccaatgtggtt

SEQ ID NO: 260 - DNA, 20 bp, Artificial Sequence
  Synthetic Oligonucleotide
gcctgtgtggccaatgtggt

SEQ ID NO: 261 - DNA, 20 bp, Artificial Sequence
  Synthetic Oligonucleotide
agcctgtgtggccaatgtgg

SEQ ID NO: 262 - DNA, 20 bp, Artificial Sequence
  Synthetic Oligonucleotide
tagcctgtgtggccaatgtg

SEQ ID NO: 263 - DNA, 20 bp, Artificial Sequence
  Synthetic Oligonucleotide
atagcctgtgtggccaatgt

SEQ ID NO: 264 - DNA, 20 bp, Artificial Sequence
  Synthetic Oligonucleotide
gatagcctgtgtggccaatg

SEQ ID NO: 265 - DNA, 20 bp, Artificial Sequence
  Synthetic Oligonucleotide
agatagcctgtgtggccaat

SEQ ID NO: 266 - DNA, 20 bp, Artificial Sequence
  Synthetic Oligonucleotide
cagatagcctgtgtggccaa

SEQ ID NO: 267 - DNA, 20 bp, Artificial Sequence
  Synthetic Oligonucleotide
tcagatagcctgtgtggcca

SEQ ID NO: 268 - DNA, 20 bp, Artificial Sequence
  Synthetic Oligonucleotide
ttcagatagcctgtgtggcc

SEQ ID NO: 269 - DNA, 20 bp, Artificial Sequence
  Synthetic Oligonucleotide
gttcagatagcctgtgtggc

SEQ ID NO: 270 - DNA, 20 bp, Artificial Sequence
  Synthetic Oligonucleotide
tgttcagatagcctgtgtgg

SEQ ID NO: 271 - DNA, 20 bp, Artificial Sequence
  Synthetic Oligonucleotide
gtgttcagatagcctgtgtg

SEQ ID NO: 272 - DNA, 20 bp, Artificial Sequence
  Synthetic Oligonucleotide
cgtgttcagatagcctgtgt

SEQ ID NO: 273 - DNA, 20 bp, Artificial Sequence
  Synthetic Oligonucleotide
ccgtgttcagatagcctgtg

SEQ ID NO: 274 - DNA, 20 bp, Artificial Sequence
  Synthetic Oligonucleotide
caccgtgttcagatagcctg

SEQ ID NO: 275 - DNA, 20 bp, Artificial Sequence
  Synthetic Oligonucleotide
tcaccgtgttcagatagcct

SEQ ID NO: 276 - DNA, 20 bp, Artificial Sequence
  Synthetic Oligonucleotide
gtcaccgtgttcagatagcc

SEQ ID NO: 277 - DNA, 20 bp, Artificial Sequence
  Synthetic Oligonucleotide
agtcaccgtgttcagatagc

SEQ ID NO: 278 - DNA, 20 bp, Artificial Sequence
  Synthetic Oligonucleotide
cagtcaccgtgttcagatag

SEQ ID NO: 279 - DNA, 20 bp, Artificial Sequence
  Synthetic Oligonucleotide
acagtcaccgtgttcagata

SEQ ID NO: 280 - DNA, 20 bp, Artificial Sequence
  Synthetic Oligonucleotide
gacagtcaccgtgttcagat

SEQ ID NO: 281 - DNA, 20 bp, Artificial Sequence
  Synthetic Oligonucleotide
agacagtcaccgtgttcaga

SEQ ID NO: 282 - DNA, 20 bp, Artificial Sequence
  Synthetic Oligonucleotide
gagacagtcaccgtgttcag

SEQ ID NO: 283 - DNA, 20 bp, Artificial Sequence
  Synthetic Oligonucleotide
agagacagtcaccgtgttca

SEQ ID NO: 284 - DNA, 20 bp, Artificial Sequence
  Synthetic Oligonucleotide
gagagacagtcaccgtgttc

SEQ ID NO: 285 - DNA, 20 bp, Artificial Sequence
  Synthetic Oligonucleotide
ggagagacagtcaccgtgtt

SEQ ID NO: 286 - DNA, 20 bp, Artificial Sequence
  Synthetic Oligonucleotide
tggagagacagtcaccgtgt

SEQ ID NO: 287 - DNA, 20 bp, Artificial Sequence
  Synthetic Oligonucleotide
ctggagagacagtcaccgtg

SEQ ID NO: 288 - DNA, 20 bp, Artificial Sequence
  Synthetic Oligonucleotide
tctggagagacagtcaccgt

SEQ ID NO: 289 - DNA, 20 bp, Homo Sapiens
actgaccagggatgagacca

SEQ ID NO: 290 - DNA, 20 bp, Homo Sapiens
tgagcagatgacccttcgtg

SEQ ID NO: 291 - DNA, 20 bp, Homo Sapiens
tggtcaaggtatggaacctg

SEQ ID NO: 292 - DNA, 20 bp, Homo Sapiens
accggcagattgtctctgga

SEQ ID NO: 293 - DNA, 20 bp, Homo Sapiens
acaggctatctgaacacggt

SEQ ID NO: 294 - DNA, 20 bp, Homo Sapiens
ccgcagttcccggacatgat

SEQ ID NO: 295 - DNA, 20 bp, Homo Sapiens
gcctctgggatctcacaacg

SEQ ID NO: 296 - DNA, 20 bp, Homo Sapiens
cttctcctctgacaaccggc

SEQ ID NO: 297 - DNA, 20 bp, Homo Sapiens
aaccggcagattgtctctgg

SEQ ID NO: 298 - DNA, 20 bp, Homo Sapiens
agcagcaaccctatcatcgt

SEQ ID NO: 299 - DNA, 20 bp, Homo Sapiens
tgactgagcagatgaccctt

SEQ ID NO: 300 - DNA, 20 bp, Homo Sapiens
gactgagcagatgacccttc

SEQ ID NO: 301 - DNA, 20 bp, Homo Sapiens
actgagcagatgacccttcg

SEQ ID NO: 302 - DNA, 20 bp, Homo Sapiens
ctgagcagatgacccttcgt

SEQ ID NO: 303 - DNA, 20 bp, Homo Sapiens
gagcagatgacccttcgtgg

SEQ ID NO: 304 - DNA, 20 bp, Homo Sapiens
agcagatgacccttcgtggc

SEQ ID NO: 305 - DNA, 20 bp, Homo Sapiens
gcagatgacccttcgtggca

SEQ ID NO: 306 - DNA, 20 bp, Homo Sapiens
cagatgacccttcgtggcac

SEQ ID NO: 307 - DNA, 20 bp, Homo Sapiens
agatgacccttcgtggcacc

SEQ ID NO: 308 - DNA, 20 bp, Homo Sapiens
gatgacccttcgtggcaccc

SEQ ID NO: 309 - DNA, 20 bp, Homo Sapiens
atgacccttcgtggcaccct

SEQ ID NO: 310 - DNA, 20 bp, Homo Sapiens
tgacccttcgtggcaccctc

SEQ ID NO: 311 - DNA, 20 bp, Homo Sapiens
gacccttcgtggcaccctca

SEQ ID NO: 312 - DNA, 20 bp, Homo Sapiens
acccttcgtggcaccctcaa

SEQ ID NO: 313 - DNA, 20 bp, Homo Sapiens
cccttcgtggcaccctcaag

SEQ ID NO: 314 - DNA, 20 bp, Homo Sapiens
ccttcgtggcaccctcaagg

SEQ ID NO: 315 - DNA, 20 bp, Homo Sapiens
cttcgtggcaccctcaaggg

SEQ ID NO: 316 - DNA, 20 bp, Homo Sapiens
ttcgtggcaccctcaagggc

SEQ ID NO: 317 - DNA, 20 bp, Homo Sapiens
tcgtggcaccctcaagggcc

SEQ ID NO: 318 - DNA, 20 bp, Homo Sapiens
cagatcgctactaccccgca

SEQ ID NO: 319 - DNA, 20 bp, Homo Sapiens
agatcgctactaccccgcag

SEQ ID NO: 320 - DNA, 20 bp, Homo Sapiens
gatcgctactaccccgcagt

SEQ ID NO: 321 - DNA, 20 bp, Homo Sapiens
atcgctactaccccgcagtt

SEQ ID NO: 322 - DNA, 20 bp, Homo Sapiens
tcgctactaccccgcagttc

SEQ ID NO: 323 - DNA, 20 bp, Homo Sapiens
cgctactaccccgcagttcc

SEQ ID NO: 324 - DNA, 20 bp, Homo Sapiens
gctactaccccgcagttccc

SEQ ID NO: 325 - DNA, 20 bp, Homo Sapiens
ctactaccccgcagttcccg

SEQ ID NO: 326 - DNA, 20 bp, Homo Sapiens
tactaccccgcagttcccgg

SEQ ID NO: 327 - DNA, 20 bp, Homo Sapiens
actaccccgcagttcccgga

SEQ ID NO: 328 - DNA, 20 bp, Homo Sapiens
ctaccccgcagttcccggac

SEQ ID NO: 329 - DNA, 20 bp, Homo Sapiens
taccccgcagttcccggaca

SEQ ID NO: 330 - DNA, 20 bp, Homo Sapiens
accccgcagttcccggacat

SEQ ID NO: 331 - DNA, 20 bp, Homo Sapiens
ccccgcagttcccggacatg

SEQ ID NO: 332 - DNA, 20 bp, Homo Sapiens
cccgcagttcccggacatga

SEQ ID NO: 333 - DNA, 20 bp, Homo Sapiens
cgcagttcccggacatgatc

SEQ ID NO: 334 - DNA, 20 bp, Homo Sapiens
gcagttcccggacatgatcc

SEQ ID NO: 335 - DNA, 20 bp, Homo Sapiens
cagttcccggacatgatcct

SEQ ID NO: 336 - DNA, 20 bp, Homo Sapiens
agttcccggacatgatcctc

SEQ ID NO: 337 - DNA, 20 bp, Homo Sapiens
gttcccggacatgatcctct

SEQ ID NO: 338 - DNA, 20 bp, Homo Sapiens
ttcccggacatgatcctctc

SEQ ID NO: 339 - DNA, 20 bp, Homo Sapiens
tcccggacatgatcctctcc

SEQ ID NO: 340 - DNA, 20 bp, Homo Sapiens
cccggacatgatcctctccg

SEQ ID NO: 341 - DNA, 20 bp, Homo Sapiens
ccggacatgatcctctccgc

SEQ ID NO: 342 - DNA, 20 bp, Homo Sapiens
cggacatgatcctctccgcc

SEQ ID NO: 343 - DNA, 20 bp, Homo Sapiens
ggacatgatcctctccgcct

SEQ ID NO: 344 - DNA, 20 bp, Homo Sapiens
gacatgatcctctccgcctc

SEQ ID NO: 345 - DNA, 20 bp, Homo Sapiens
acatgatcctctccgcctct

SEQ ID NO: 346 - DNA, 20 bp, Homo Sapiens
catgatcctctccgcctctc

SEQ ID NO: 347 - DNA, 20 bp, Homo Sapiens
atgatcctctccgcctctcg

SEQ ID NO: 348 - DNA, 20 bp, Homo Sapiens
catcatcatgtggaaactga

SEQ ID NO: 349 - DNA, 20 bp, Homo Sapiens
atcatcatgtggaaactgac

SEQ ID NO: 350 - DNA, 20 bp, Homo Sapiens
tcatcatgtggaaactgacc

SEQ ID NO: 351 - DNA, 20 bp, Homo Sapiens
catcatgtggaaactgacca

SEQ ID NO: 352 - DNA, 20 bp, Homo Sapiens
atcatgtggaaactgaccag

SEQ ID NO: 353 - DNA, 20 bp, Homo Sapiens
tcatgtggaaactgaccagg

SEQ ID NO: 354 - DNA, 20 bp, Homo Sapiens
catgtggaaactgaccaggg

SEQ ID NO: 355 - DNA, 20 bp, Homo Sapiens
atgtggaaactgaccaggga

SEQ ID NO: 356 - DNA, 20 bp, Homo Sapiens
tgtggaaactgaccagggat

SEQ ID NO: 357 - DNA, 20 bp, Homo Sapiens
gtggaaactgaccagggatg

SEQ ID NO: 358 - DNA, 20 bp, Homo Sapiens
tggaaactgaccagggatga

SEQ ID NO: 359 - DNA, 20 bp, Homo Sapiens
ggaaactgaccagggatgag

SEQ ID NO: 360 - DNA, 20 bp, Homo Sapiens
gaaactgaccagggatgaga

SEQ ID NO: 361 - DNA, 20 bp, Homo Sapiens
gggatggaaccctgcgcctc

SEQ ID NO: 362 - DNA, 20 bp, Homo Sapiens
ggatggaaccctgcgcctct

SEQ ID NO: 363 - DNA, 20 bp, Homo Sapiens
gatggaaccctgcgcctctg

SEQ ID NO: 364 - DNA, 20 bp, Homo Sapiens
atggaaccctgcgcctctgg

SEQ ID NO: 365 - DNA, 20 bp, Homo Sapiens
cctctgggatctcacaacgg

SEQ ID NO: 366 - DNA, 20 bp, Homo Sapiens
ctctgggatctcacaacggg

SEQ ID NO: 367 - DNA, 20 bp, Homo Sapiens
tctgggatctcacaacgggc

SEQ ID NO: 368 - DNA, 20 bp, Homo Sapiens
ctgggatctcacaacgggca

SEQ ID NO: 369 - DNA, 20 bp, Homo Sapiens
tgggatctcacaacgggcac

SEQ ID NO: 370 - DNA, 20 bp, Homo Sapiens
gggatctcacaacgggcacc

SEQ ID NO: 371 - DNA, 20 bp, Homo Sapiens
ggatctcacaacgggcacca

SEQ ID NO: 372 - DNA, 20 bp, Homo Sapiens
gatctcacaacgggcaccac

SEQ ID NO: 373 - DNA, 20 bp, Homo Sapiens
atctcacaacgggcaccacc

SEQ ID NO: 374 - DNA, 20 bp, Homo Sapiens
tctcacaacgggcaccacca

SEQ ID NO: 375 - DNA, 20 bp, Homo Sapiens
ctcacaacgggcaccaccac

SEQ ID NO: 376 - DNA, 20 bp, Homo Sapiens
tcacaacgggcaccaccacg

SEQ ID NO: 377 - DNA, 20 bp, Homo Sapiens
cacaacgggcaccaccacga

SEQ ID NO: 378 - DNA, 20 bp, Homo Sapiens
acaacgggcaccaccacgag

SEQ ID NO: 379 - DNA, 20 bp, Homo Sapiens
caacgggcaccaccacgagg

SEQ ID NO: 380 - DNA, 20 bp, Homo Sapiens
tgtgctgagtgtggccttct

SEQ ID NO: 381 - DNA, 20 bp, Homo Sapiens
gtgctgagtgtggccttctc

SEQ ID NO: 382 - DNA, 20 bp, Homo Sapiens
tgctgagtgtggccttctcc

SEQ ID NO: 383 - DNA, 20 bp, Homo Sapiens
gctgagtgtggccttctcct

SEQ ID NO: 384 - DNA, 20 bp, Homo Sapiens
ctgagtgtggccttctcctc

SEQ ID NO: 385 - DNA, 20 bp, Homo Sapiens
tgagtgtggccttctcctct

SEQ ID NO: 386 - DNA, 20 bp, Homo Sapiens
gagtgtggccttctcctctg

SEQ ID NO: 387 - DNA, 20 bp, Homo Sapiens
agtgtggccttctcctctga

SEQ ID NO: 388 - DNA, 20 bp, Homo Sapiens
gtgtggccttctcctctgac

SEQ ID NO: 389 - DNA, 20 bp, Homo Sapiens
tgtggccttctcctctgaca

SEQ ID NO: 390 - DNA, 20 bp, Homo Sapiens
gtggccttctcctctgacaa

SEQ ID NO: 391 - DNA, 20 bp, Homo Sapiens
tggccttctcctctgacaac

SEQ ID NO: 392 - DNA, 20 bp, Homo Sapiens
ggccttctcctctgacaacc

SEQ ID NO: 393 - DNA, 20 bp, Homo Sapiens
gccttctcctctgacaaccg

SEQ ID NO: 394 - DNA, 20 bp, Homo Sapiens
ccttctcctctgacaaccgg

SEQ ID NO: 395 - DNA, 20 bp, Homo Sapiens
ttctcctctgacaaccggca

SEQ ID NO: 396 - DNA, 20 bp, Homo Sapiens
tctcctctgacaaccggcag

SEQ ID NO: 397 - DNA, 20 bp, Homo Sapiens
ctcctctgacaaccggcaga

SEQ ID NO: 398 - DNA, 20 bp, Homo Sapiens
tcctctgacaaccggcagat

SEQ ID NO: 399 - DNA, 20 bp, Homo Sapiens
cctctgacaaccggcagatt

SEQ ID NO: 400 - DNA, 20 bp, Homo Sapiens
ctctgacaaccggcagattg

SEQ ID NO: 401 - DNA, 20 bp, Homo Sapiens
tctgacaaccggcagattgt

SEQ ID NO: 402 - DNA, 20 bp, Homo Sapiens
ctgacaaccggcagattgtc

SEQ ID NO: 403 - DNA, 20 bp, Homo Sapiens
tgacaaccggcagattgtct

SEQ ID NO: 404 - DNA, 20 bp, Homo Sapiens
gacaaccggcagattgtctc

SEQ ID NO: 405 - DNA, 20 bp, Homo Sapiens
acaaccggcagattgtctct

SEQ ID NO: 406 - DNA, 20 bp, Homo Sapiens
caaccggcagattgtctctg

SEQ ID NO: 407 - DNA, 20 bp, Homo Sapiens
ccggcagattgtctctggat

SEQ ID NO: 408 - DNA, 20 bp, Homo Sapiens
cggcagattgtctctggatc

SEQ ID NO: 409 - DNA, 20 bp, Homo Sapiens
ggcagattgtctctggatct

SEQ ID NO: 410 - DNA, 20 bp, Homo Sapiens
gcagattgtctctggatctc

SEQ ID NO: 411 - DNA, 20 bp, Homo Sapiens
cagattgtctctggatctcg

SEQ ID NO: 412 - DNA, 20 bp, Homo Sapiens
agattgtctctggatctcga

SEQ ID NO: 413 - DNA, 20 bp, Homo Sapiens
gattgtctctggatctcgag

SEQ ID NO: 414 - DNA, 20 bp, Homo Sapiens
attgtctctggatctcgaga

SEQ ID NO: 415 - DNA, 20 bp, Homo Sapiens
ttgtctctggatctcgagat

SEQ ID NO: 416 - DNA, 20 bp, Homo Sapiens
tgtctctggatctcgagata

SEQ ID NO: 417 - DNA, 20 bp, Homo Sapiens
gtctctggatctcgagataa

SEQ ID NO: 418 - DNA, 20 bp, Homo Sapiens
tctctggatctcgagataaa

SEQ ID NO: 419 - DNA, 20 bp, Homo Sapiens
ctctggatctcgagataaaa

SEQ ID NO: 420 - DNA, 20 bp, Homo Sapiens
tctggatctcgagataaaac

SEQ ID NO: 421 - DNA, 20 bp, Homo Sapiens
ctggatctcgagataaaacc

SEQ ID NO: 422 - DNA, 20 bp, Homo Sapiens
cgcttctcgcccaacagcag

SEQ ID NO: 423 - DNA, 20 bp, Homo Sapiens
gcttctcgcccaacagcagc

SEQ ID NO: 424 - DNA, 20 bp, Homo Sapiens
cttctcgcccaacagcagca

SEQ ID NO: 425 - DNA, 20 bp, Homo Sapiens
ttctcgcccaacagcagcaa

SEQ ID NO: 426 - DNA, 20 bp, Homo Sapiens
tctcgcccaacagcagcaac

SEQ ID NO: 427 - DNA, 20 bp, Homo Sapiens
ctcgcccaacagcagcaacc

SEQ ID NO: 428 - DNA, 20 bp, Homo Sapiens
tcgcccaacagcagcaaccc

SEQ ID NO: 429 - DNA, 20 bp, Homo Sapiens
cgcccaacagcagcaaccct

SEQ ID NO: 430 - DNA, 20 bp, Homo Sapiens
gcccaacagcagcaacccta

SEQ ID NO: 431 - DNA, 20 bp, Homo Sapiens
cccaacagcagcaaccctat

SEQ ID NO: 432 - DNA, 20 bp, Homo Sapiens
ccaacagcagcaaccctatc

SEQ ID NO: 433 - DNA, 20 bp, Homo Sapiens
caacagcagcaaccctatca

SEQ ID NO: 434 - DNA, 20 bp, Homo Sapiens
aacagcagcaaccctatcat

SEQ ID NO: 435 - DNA, 20 bp, Homo Sapiens
acagcagcaaccctatcatc

SEQ ID NO: 436 - DNA, 20 bp, Homo Sapiens
cagcagcaaccctatcatcg

SEQ ID NO: 437 - DNA, 20 bp, Homo Sapiens
gcagcaaccctatcatcgtc

SEQ ID NO: 438 - DNA, 20 bp, Homo Sapiens
cagcaaccctatcatcgtct

SEQ ID NO: 439 - DNA, 20 bp, Homo Sapiens
agcaaccctatcatcgtctc

SEQ ID NO: 440 - DNA, 20 bp, Homo Sapiens
gcaaccctatcatcgtctcc

SEQ ID NO: 441 - DNA, 20 bp, Homo Sapiens
caaccctatcatcgtctcct

SEQ ID NO: 442 - DNA, 20 bp, Homo Sapiens
aaccctatcatcgtctcctg

SEQ ID NO: 443 - DNA, 20 bp, Homo Sapiens
accctatcatcgtctcctgt

SEQ ID NO: 444 - DNA, 20 bp, Homo Sapiens
ccctatcatcgtctcctgtg

SEQ ID NO: 445 - DNA, 20 bp, Homo Sapiens
cctatcatcgtctcctgtgg

SEQ ID NO: 446 - DNA, 20 bp, Homo Sapiens
ctatcatcgtctcctgtggc

SEQ ID NO: 447 - DNA, 20 bp, Homo Sapiens
tatcatcgtctcctgtggct

SEQ ID NO: 448 - DNA, 20 bp, Homo Sapiens
atcatcgtctcctgtggctg

SEQ ID NO: 449 - DNA, 20 bp, Homo Sapiens
tcatcgtctcctgtggctgg

SEQ ID NO: 450 - DNA, 20 bp, Homo Sapiens
catcgtctcctgtggctggg

SEQ ID NO: 451 - DNA, 20 bp, Homo Sapiens
atcgtctcctgtggctggga

SEQ ID NO: 452 - DNA, 20 bp, Homo Sapiens
gtggctgggacaagctggtc

SEQ ID NO: 453 - DNA, 20 bp, Homo Sapiens
tggctgggacaagctggtca

SEQ ID NO: 454 - DNA, 20 bp, Homo Sapiens
ggctgggacaagctggtcaa

SEQ ID NO: 455 - DNA, 20 bp, Homo Sapiens
gctgggacaagctggtcaag

SEQ ID NO: 456 - DNA, 20 bp, Homo Sapiens
ctgggacaagctggtcaagg

SEQ ID NO: 457 - DNA, 20 bp, Homo Sapiens
tgggacaagctggtcaaggt

SEQ ID NO: 458 - DNA, 20 bp, Homo Sapiens
gggacaagctggtcaaggta

SEQ ID NO: 459 - DNA, 20 bp, Homo Sapiens
ggacaagctggtcaaggtat

SEQ ID NO: 460 - DNA, 20 bp, Homo Sapiens
gacaagctggtcaaggtatg

SEQ ID NO: 461 - DNA, 20 bp, Homo Sapiens
acaagctggtcaaggtatgg

SEQ ID NO: 462 - DNA, 20 bp, Homo Sapiens
caagctggtcaaggtatgga

SEQ ID NO: 463 - DNA, 20 bp, Homo Sapiens
aagctggtcaaggtatggaa

SEQ ID NO: 464 - DNA, 20 bp, Homo Sapiens
agctggtcaaggtatggaac

SEQ ID NO: 465 - DNA, 20 bp, Homo Sapiens
gctggtcaaggtatggaacc

SEQ ID NO: 466 - DNA, 20 bp, Homo Sapiens
ctggtcaaggtatggaacct

SEQ ID NO: 467 - DNA, 20 bp, Homo Sapiens
gaacctggctaactgcaagc

SEQ ID NO: 468 - DNA, 20 bp, Homo Sapiens
aacctggctaactgcaagct

SEQ ID NO: 469 - DNA, 20 bp, Homo Sapiens
acctggctaactgcaagctg

SEQ ID NO: 470 - DNA, 20 bp, Homo Sapiens
aaccacattggccacacagg

SEQ ID NO: 471 - DNA, 20 bp, Homo Sapiens
accacattggccacacaggc

SEQ ID NO: 472 - DNA, 20 bp, Homo Sapiens
ccacattggccacacaggct

SEQ ID NO: 473 - DNA, 20 bp, Homo Sapiens
cacattggccacacaggcta

SEQ ID NO: 474 - DNA, 20 bp, Homo Sapiens
acattggccacacaggctat

SEQ ID NO: 475 - DNA, 20 bp, Homo Sapiens
cattggccacacaggctatc

SEQ ID NO: 476 - DNA, 20 bp, Homo Sapiens
attggccacacaggctatct

SEQ ID NO: 477 - DNA, 20 bp, Homo Sapiens
ttggccacacaggctatctg

SEQ ID NO: 478 - DNA, 20 bp, Homo Sapiens
tggccacacaggctatctga

SEQ ID NO: 479 - DNA, 20 bp, Homo Sapiens
ggccacacaggctatctgaa

SEQ ID NO: 480 - DNA, 20 bp, Homo Sapiens
gccacacaggctatctgaac

SEQ ID NO: 481 - DNA, 20 bp, Homo Sapiens
ccacacaggctatctgaaca

SEQ ID NO: 482 - DNA, 20 bp, Homo Sapiens
cacacaggctatctgaacac

SEQ ID NO: 483 - DNA, 20 bp, Homo Sapiens
acacaggctatctgaacacg

SEQ ID NO: 484 - DNA, 20 bp, Homo Sapiens
cacaggctatctgaacacgg

SEQ ID NO: 485 - DNA, 20 bp, Homo Sapiens
caggctatctgaacacggtg

SEQ ID NO: 486 - DNA, 20 bp, Homo Sapiens
aggctatctgaacacggtga

SEQ ID NO: 487 - DNA, 20 bp, Homo Sapiens
ggctatctgaacacggtgac

SEQ ID NO: 488 - DNA, 20 bp, Homo Sapiens
gctatctgaacacggtgact

SEQ ID NO: 489 - DNA, 20 bp, Homo Sapiens
ctatctgaacacggtgactg

SEQ ID NO: 490 - DNA, 20 bp, Homo Sapiens
tatctgaacacggtgactgt

SEQ ID NO: 491 - DNA, 20 bp, Homo Sapiens
atctgaacacggtgactgtc

SEQ ID NO: 492 - DNA, 20 bp, Homo Sapiens
tctgaacacggtgactgtct

SEQ ID NO: 493 - DNA, 20 bp, Homo Sapiens
ctgaacacggtgactgtctc

SEQ ID NO: 494 - DNA, 20 bp, Homo Sapiens
tgaacacggtgactgtctct

SEQ ID NO: 495 - DNA, 20 bp, Homo Sapiens
gaacacggtgactgtctctc

SEQ ID NO: 496 - DNA, 20 bp, Homo Sapiens
aacacggtgactgtctctcc

SEQ ID NO: 497 - DNA, 20 bp, Homo Sapiens
acacggtgactgtctctcca

SEQ ID NO: 498 - DNA, 20 bp, Homo Sapiens
cacggtgactgtctctccag

SEQ ID NO: 499 - DNA, 20 bp, Homo Sapiens
acggtgactgtctctccaga

SEQ ID NO: 500 - DNA, 1140 bp, Homo Sapiens
ctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacctccctggcctggtctgctgatggccagactctgtttgctggctacacggacaacctggtgcgagtgtggcaggtgaccattggcacacgctagaagtttatggcagagctttacaaataaaaaaaaaactggcttttctgacttttaggtttttttttcttatatgcaaaaaa

SEQ ID NO: 501 - RNA, 19 nt, Artificial Sequence
  Synthetic Construct
ccuuuacacgcuagauggu

SEQ ID NO: 502 - RNA, 18 nt, Artificial Sequence
  Synthetic Construct
accaucuagcguguamgg